SEQUENCE LISTING (117 sequences)

SEQ ID NO: 1 - DNA, 1098 bp, Trichoderma hamatum
  sig_peptide 1..57
  mat_peptide 58..1095
  source 1..1098: mol_type = genomic DNA
  CDS 1..64
  CDS 136..795
  CDS 857..1095
atgaagctctcaaatgctgtggccgtaagcctcgtctccctaccaggggccatggcttggggaggtgtgtattgactccctcgtgatgcgccatcattatcacttgccttctaacatcagctctcttcgaatcaggtctgggacacgtaacgacagcctttgttgcaagcaactttatcgccaatacgacagaagcctacctaaagcagctccttggtagccaagatgccgactacatggccaaggtagcctcctgggccgactcgatcagatacacaaaatggggtagattcacaagcacgtttcatttcatcgatgcgcacgacagtcctcccgaggactgcaatgtcgactttgagcgcgattgtaaagagaccggctgcgtcatcaccgctctggccaactacacacaacagtccgtcgacccgtccctaccggcatggcgccgagcgcaggcagccaagtttgtcattcactttgtcggcgatctgcaccagccgctacataacgaggatgttgcaagaggtggaaacggtattcacgtgctgtggaatggaaaagaattcaacctgcatcacgtctgggatagctccattacagagaaatggcttggcatgcgtggtagaaaaccatatagctgggcggagaagtggtccaaagatctcacacagaagatcaacagcggcatatacgcagacgagaaggacgattggttgaaggacttggactttagcaaccccgaagagacggcattggcttggtcacgagaatgtaacaaacttgtgtgcgagtatggtaagtcaagcccaaatctgcgccagaccctagctttgtcactaacctggtgaatttatagtattccccgagggtcccaaggccattgctggccaagagttgagcggcgagtactacgaaaaggcggctcctgtgctcgaaaagcaagtcgcccgtgccggataccgaatggcagcttggttggacaaggttgtcgaccaatacctaaagttggaagcaagctacacaggagagctgcccaccgaagactatatggaagagcctttggatgaatttatggaggagccaattggagagttataa

SEQ ID NO: 2 - protein, 321 aa, Trichoderma hamatum
MKLSNAVAVSLVSLPGAMAWGGLGHVTTAFVASNFIANTTEAYLKQLLGSQDADYMAKVASWADSIRYTKWGRFTSTFHFIDAHDSPPEDCNVDFERDCKETGCVITALANYTQQSVDPSLPAWRRAQAAKFVIHFVGDLHQPLHNEDVARGGNGIHVLWNGKEFNLHHVWDSSITEKWLGMRGRKPYSWAEKWSKDLTQKINSGIYADEKDDWLKDLDFSNPEETALAWSRECNKLVCEYVFPEGPKAIAGQELSGEYYEKAAPVLEKQVARAGYRMAAWLDKVVDQYLKLEASYTGELPTEDYMEEPLDEFMEEPIGEL

SEQ ID NO: 3 - protein, 302 aa, Trichoderma hamatum
WGGLGHVTTAFVASNFIANTTEAYLKQLLGSQDADYMAKVASWADSIRYTKWGRFTSTFHFIDAHDSPPEDCNVDFERDCKETGCVITALANYTQQSVDPSLPAWRRAQAAKFVIHFVGDLHQPLHNEDVARGGNGIHVLWNGKEFNLHHVWDSSITEKWLGMRGRKPYSWAEKWSKDLTQKINSGIYADEKDDWLKDLDFSNPEETALAWSRECNKLVCEYVFPEGPKAIAGQELSGEYYEKAAPVLEKQVARAGYRMAAWLDKVVDQYLKLEASYTGELPTEDYMEEPLDEFMEEPIGEL

SEQ ID NO: 4 - DNA, 1138 bp, Morchella costata
  sig_peptide 1..63
  mat_peptide 64..1135
  source 1..1138: mol_type = genomic DNA
  CDS 1..339
  CDS 404..1135
atgcataccagcaccctccccctcctcctcctccccgccgtcttcgccccctccgcctcggcctggggcatgctcggccaccgcaccgtcgccctcctgtcgacgcgctatctcctccccgagaccgcagtcttcgtaaagtccctcctcccaaagccccagaccatcgtctcggcctccacctggcccgactactacgcacacacgcccgacggccgctactccgcgccctggcactggatcgactcccacgataacccgccgcacacctgcgaggtcaactaccgccgtgactgcagcggcgacgagggctgcatcgtcagtgccatcgccaacatggtacgtctcagggttagggttggtcgtggaattatggatatggatatgctaacacgcacaacagacatcgcgggtgacggacgaggagctcccgttctacgagcgccagatggcgctaaagttcatcatccactttgtcggcgacatccaccagccgctgcacacggaggaccttctgcgcggcggcaaccagatccccgtgctgtggggcaagcagcacacgaacctgcaccacgtgtgggactcgagcatcgccgagaagcaccgcggcgggagcgcggtgcgccacgcggtcggctgggcggacgagctgcacgccgagcttgagcacgggaggtacagcgagctgcgcgccgggtggggctcgtgcgtcgacccgcgcacggccgaggagtgcgcgctcgcgtgggcgggcgaggccaaccgctggatgtgcgactatgtgctcccgcagacgtaccccgagggcctggagggcgtggatgtcagtggcgagtactacgagggggcgagggaggtcgtggacgtgttggttgcgaaggcggggtggaggctcgccggctacctgaacatgattgtgacggggagcacggggctggaggatggcgagagggcagcgacggccgcggtggaggaggattgggtgagctttgacagagaggaggagaggaagtatgcagtggcagcagcagcggaggaggaggaggaggaggcagcttcaggacacgagctgaagaagagggtgatgaacaaggccaatagcggctggtggggcaatgttagaactaacggcaactga

SEQ ID NO: 5 - protein, 357 aa, Morchella costata
MHTSTLPLLLLPAVFAPSASAWGMLGHRTVALLSTRYLLPETAVFVKSLLPKPQTIVSASTWPDYYAHTPDGRYSAPWHWIDSHDNPPHTCEVNYRRDCSGDEGCIVSAIANMTSRVTDEELPFYERQMALKFIIHFVGDIHQPLHTEDLLRGGNQIPVLWGKQHTNLHHVWDSSIAEKHRGGSAVRHAVGWADELHAELEHGRYSELRAGWGSCVDPRTAEECALAWAGEANRWMCDYVLPQTYPEGLEGVDVSGEYYEGAREVVDVLVAKAGWRLAGYLNMIVTGSTGLEDGERAATAAVEEDWVSFDREEERKYAVAAAAEEEEEEAASGHELKKRVMNKANSGWWGNVRTNGN

SEQ ID NO: 6 - protein, 336 aa, Morchella costata
WGMLGHRTVALLSTRYLLPETAVFVKSLLPKPQTIVSASTWPDYYAHTPDGRYSAPWHWIDSHDNPPHTCEVNYRRDCSGDEGCIVSAIANMTSRVTDEELPFYERQMALKFIIHFVGDIHQPLHTEDLLRGGNQIPVLWGKQHTNLHHVWDSSIAEKHRGGSAVRHAVGWADELHAELEHGRYSELRAGWGSCVDPRTAEECALAWAGEANRWMCDYVLPQTYPEGLEGVDVSGEYYEGAREVVDVLVAKAGWRLAGYLNMIVTGSTGLEDGERAATAAVEEDWVSFDREEERKYAVAAAAEEEEEEAASGHELKKRVMNKANSGWWGNVRTNGN

SEQ ID NO: 7 - DNA, 1116 bp, Trichoderma reesei
  sig_peptide 1..60
  mat_peptide 61..1113
  source 1..1116: mol_type = genomic DNA
  CDS 1..131
  CDS 213..1113
atgccttccatgtcaaagattgccttggcgactctggccacgctccagggcgcccacgcctggggagtcctgggccacgcaaccgtcgcctacgtggcccagcactatctcaacgcagccacggcctcatggtgagcagactgacacttattcatcactgatgatgatgaagatgacgacaagttcatccactgacaatcttaatacttcagggcccagggagtcctcaacgacacgtccagctcgtacctggccagcatcgcctcctgggccgacacctaccgcaccaccgcggccggcaagttctcggcgcccttccacttcatcgacgcccaggacagcccgcccagctcgtgcaacgtcgactacgaccgcgactgcggcagcgccggctgctccatctccgccatggccaactacacccagcgcgtgggcgacggccggctgtccgccgccaacgtcgccgaggcgctcaagttcctggtgcactttgtcggcgacatgacgcagccgctgcacgacgaggcgtacgaggtcggcggcaacgacattgccgtcaagttccagggctacaacgacaacctgcacgccgactgggacacgtacatccccgaggcgctcatcggcggcgactcgctggcggatgcccagtcctgggccagctcgctcgtcagcgacatcgcctcgggcgcgtacaagtcgcaggccgcgggctggatcaagggcgacaccctcggcgatgtcatcggcacggccacgcgctgggccagcgacgcaaacgccctcgtctgcaccgtcgtcatgcccaacggcgtggcggccctgcagcagggcgacctgtacccgacctactacgacgccgtcatcggcaccgtcgagctgcagattgcaaagggcggctaccgcctggccaactggctcaacatggtgtacgcgtccaacattgcgaagcgcgaggcgggctctgtcgacgctcgggatgcttcgcctctgcctgacctcttggggcgagacttgctgccggctgctcgccctgctacccgcgctcagcttgcgagggcggccatggagggttcgtgttgcggaagtggtcgggagcacgagcacaagcactag

SEQ ID NO: 8 - protein, 344 aa, Trichoderma reesei
MPSMSKIALATLATLQGAHAWGVLGHATVAYVAQHYLNAATASWAQGVLNDTSSSYLASIASWADTYRTTAAGKFSAPFHFIDAQDSPPSSCNVDYDRDCGSAGCSISAMANYTQRVGDGRLSAANVAEALKFLVHFVGDMTQPLHDEAYEVGGNDIAVKFQGYNDNLHADWDTYIPEALIGGDSLADAQSWASSLVSDIASGAYKSQAAGWIKGDTLGDVIGTATRWASDANALVCTVVMPNGVAALQQGDLYPTYYDAVIGTVELQIAKGGYRLANWLNMVYASNIAKREAGSVDARDASPLPDLLGRDLLPAARPATRAQLARAAMEGSCCGSGREHEHKH

SEQ ID NO: 9 - protein, 324 aa, Trichoderma reesei
WGVLGHATVAYVAQHYLNAATASWAQGVLNDTSSSYLASIASWADTYRTTAAGKFSAPFHFIDAQDSPPSSCNVDYDRDCGSAGCSISAMANYTQRVGDGRLSAANVAEALKFLVHFVGDMTQPLHDEAYEVGGNDIAVKFQGYNDNLHADWDTYIPEALIGGDSLADAQSWASSLVSDIASGAYKSQAAGWIKGDTLGDVIGTATRWASDANALVCTVVMPNGVAALQQGDLYPTYYDAVIGTVELQIAKGGYRLANWLNMVYASNIAKREAGSVDARDASPLPDLLGRDLLPAARPATRAQLARAAMEGSCCGSGREHEHKH

SEQ ID NO: 10 - DNA, 1081 bp, Penicillium cremeogriseum
  sig_peptide 1..60
  mat_peptide 61..1078
  source 1..1081: mol_type = genomic DNA
  CDS 1..131
  CDS 184..1078
atggcacctctgtccaaggttgctctcgtcacccttggctccttgagcggagtaaatgcatggggtgtgcttggccacgcaaccgttgcctacgtcgcgcagaactacctttcgtctggcactgcatcatggtgagggtgaaagaaatccccgaggccgcgatggatactaatgattttcaagggcgcaaggcgttcttggagacacttccagctcgtacctagccaacatcgcctcttgggctgaccagtaccgtgcgacaaccgctggcaagtggtccgcgccgcttcacttcatcgacgccgaggacaatcctcccaccagctgcaacgtggattacactcgcgactgcggtagctccggctgctccatctctgcaattgcaaactacactcaacgtgcaggtgatgcccggctcagcactgctcaaatcgctgaagctgtcaagttcctggtccacttcctgggagacgttactcagcccctgcacgatgaggcgtacgaggttggcggaaacagcatcaaggtcactttcgatggttatagcgacaacctccatgccgactgggatacctacatgccccagaaactgatcggcggcagcacgctctccgatgcacagtcttgggctaagacgctcgtcggtgagatcgactcgggaagctacaagtctcaagcagcaagctggattcagggagacactatcagtgatcctattactaccgccacccgatgggcgtctgatgcgaatgcctttgtctgcactgttgtcatgccagatggagcttctgccttggagactggtgatctttaccctacctactacaattctgctattggtaccatcgagctgcagattgcaaagggtggataccgcctggccaactggctcaacatgatctacaacaatgatatcgccaagcgagacctggagggctttgtcaacgaggctcgaagtgaaggtctgccggatctcatgggtcgcgacctgctgcccgagcctcgcgaattgagccgcgctcagttggccagagaggctatgggaggagattgctgcggctcctcgagacatgctcactga

SEQ ID NO: 11 - protein, 342 aa, Penicillium cremeogriseum
MAPLSKVALVTLGSLSGVNAWGVLGHATVAYVAQNYLSSGTASWAQGVLGDTSSSYLANIASWADQYRATTAGKWSAPLHFIDAEDNPPTSCNVDYTRDCGSSGCSISAIANYTQRAGDARLSTAQIAEAVKFLVHFLGDVTQPLHDEAYEVGGNSIKVTFDGYSDNLHADWDTYMPQKLIGGSTLSDAQSWAKTLVGEIDSGSYKSQAASWIQGDTISDPITTATRWASDANAFVCTVVMPDGASALETGDLYPTYYNSAIGTIELQIAKGGYRLANWLNMIYNNDIAKRDLEGFVNEARSEGLPDLMGRDLLPEPRELSRAQLAREAMGGDCCGSSRHAH

SEQ ID NO: 12 - protein, 322 aa, Penicillium cremeogriseum
WGVLGHATVAYVAQNYLSSGTASWAQGVLGDTSSSYLANIASWADQYRATTAGKWSAPLHFIDAEDNPPTSCNVDYTRDCGSSGCSISAIANYTQRAGDARLSTAQIAEAVKFLVHFLGDVTQPLHDEAYEVGGNSIKVTFDGYSDNLHADWDTYMPQKLIGGSTLSDAQSWAKTLVGEIDSGSYKSQAASWIQGDTISDPITTATRWASDANAFVCTVVMPDGASALETGDLYPTYYNSAIGTIELQIAKGGYRLANWLNMIYNNDIAKRDLEGFVNEARSEGLPDLMGRDLLPEPRELSRAQLAREAMGGDCCGSSRHAH

SEQ ID NO: 13 - DNA, 1127 bp, Stenocarpella maydis
  sig_peptide 1..57
  mat_peptide 58..1124
  source 1..1127: mol_type = genomic DNA
  CDS 1..64
  CDS 142..801
  CDS 964..1124
atgaagctgtctggtgtcgccgcgttgggccttgtagctgtcccggaagtgctggcatggggaggtgagcagccgtcccgcgctgtgccccagcaccaagacaggtgcatcgttcgccctttattaacgttagctgcccaggctttgggcacatcacgatcgcatacgttgctagcaactttgtccagccgcagacgacagcctacttccaaaccctcctccggaatgacacagccgactacctcgccaacgtggctacgtgggccgactcgatccgctatacgaaatggggccactatactggcgtgttccacttcatcgacgccaaagacgaaccgcccaacaactgctccgtcgagctcgaccgcgactgcaaggaggagggctgcgtcgtaacggcgatccaaaactacacctcgcagatcctcgacccctccattccagtctggctccgcgcgcaggccgccaaatttgtggtgcactttgtcggcgacatccaccagcccctgcacgacgaaaacgtgtccaggggcgggaatgggatccacgtgctgtgggaggggcgggagctgaacctccaccacgtctgggacagctctatcgcggagaagtgggtgggcggcgtcaggaggaagccataccatggggcgcatgagtgggctcaggaactcacgggcaagatcaaagacggccaattcaaggcccacagcaagacttggctggacggcgtggactttgctgatcccatcaagaccgccctgggttgggcgagggaaggaaattcatttgtctgttcgcacggtgagcgtctctcgtttccgctcgttattttctgttttctgactggatctcgggccgtcgttggtctggagatccagcacgggcctcaatccacacttgttcctacccttcatttcgtggggctctgtcgaccgttcgagcaatttgctaacgcatgtctagtgcttcccgaggggccagaagccatcgtgggacaggaactaggcggggagtactatgaaaaggctgcgccggtgattgagctccaggtcgcccgtgcgggattccgtcttgcggcgtggctcgacttgatcgtggcggcgctcaaggtcgagtctgagttatga

SEQ ID NO: 14 - protein, 295 aa, Stenocarpella maydis
MKLSGVAALGLVAVPEVLAWGGFGHITIAYVASNFVQPQTTAYFQTLLRNDTADYLANVATWADSIRYTKWGHYTGVFHFIDAKDEPPNNCSVELDRDCKEEGCVVTAIQNYTSQILDPSIPVWLRAQAAKFVVHFVGDIHQPLHDENVSRGGNGIHVLWEGRELNLHHVWDSSIAEKWVGGVRRKPYHGAHEWAQELTGKIKDGQFKAHSKTWLDGVDFADPIKTALGWAREGNSFVCSHVLPEGPEAIVGQELGGEYYEKAAPVIELQVARAGFRLAAWLDLIVAALKVESEL

SEQ ID NO: 15 - protein, 276 aa, Stenocarpella maydis
WGGFGHITIAYVASNFVQPQTTAYFQTLLRNDTADYLANVATWADSIRYTKWGHYTGVFHFIDAKDEPPNNCSVELDRDCKEEGCVVTAIQNYTSQILDPSIPVWLRAQAAKFVVHFVGDIHQPLHDENVSRGGNGIHVLWEGRELNLHHVWDSSIAEKWVGGVRRKPYHGAHEWAQELTGKIKDGQFKAHSKTWLDGVDFADPIKTALGWAREGNSFVCSHVLPEGPEAIVGQELGGEYYEKAAPVIELQVARAGFRLAAWLDLIVAALKVESEL

SEQ ID NO: 16 - DNA, 1086 bp, Stenocarpella maydis
  sig_peptide 1..51
  mat_peptide 52..1083
  source 1..1086: mol_type = genomic DNA
  CDS 1..122
  CDS 192..1083
atggcctcgaatctcctcctaacacttgccgccttgcaaggcgtgagggcatggggcactctcgggcatgccaccatcgcctatatcgcacagaactacgttacagacgaagtagcttcctggtacgtctgatttacaaagatgcctcaccagcgtgcaagtacacgaacttaccagctttgtcaccgcagggctcaaggtgtgctcagtgacagctccgattcctacctggccaacatcgcctcgtgggctgacagctaccgcacaacgactgccggcgcatggtctgcccctctgcatttcattgacgccgaagacagcccgcctagtgactgcaacgtggattatgaccgggactgtggatcgacgggctgctccgtctccgccattgccaactacacccagcgtgtcggtgatggccgcctttccgctgcaaacgttgccgaagccctcaagttcctggtccacttcactggagacatcacgcagcccctgcatgatgaggcttacgagaagggagccaacggtgtcgacgtcacttatcagggctactccgacaacctccatgccgactgggacacatacctccccgcgacgctcgtcggtggttccacccttgctgatgccaaggcctgggcaaccaacctcaccgctgagatcaactcgggcatctacaagtcccaggcggcaggctggattgcgggcgatagcctgagcgagcccatagataccgccatggcctgggccaccgacgccaacgcctacgtctgcaccgtcgtgatgcccaacggcgctgcggccctgacggccatgaaggatctgtaccctacatactacgagtcggtcatccccactatcgagcttcagatagccaagggtggctatcgccttgggaactggttgaacctgatctataatgccagcattaagcggcgagatcttgtttctggcgaaggcatcaagagggccgagaagaagccgctgccggatctgatgggccgtgacctccttccggcgccgaggccattgagcaaggcgaagcttgccagggctgctttcggctatggctgtaaccatgagcactag

SEQ ID NO: 17 - protein, 338 aa, Stenocarpella maydis
MASNLLLTLAALQGVRAWGTLGHATIAYIAQNYVTDEVASWAQGVLSDSSDSYLANIASWADSYRTTTAGAWSAPLHFIDAEDSPPSDCNVDYDRDCGSTGCSVSAIANYTQRVGDGRLSAANVAEALKFLVHFTGDITQPLHDEAYEKGANGVDVTYQGYSDNLHADWDTYLPATLVGGSTLADAKAWATNLTAEINSGIYKSQAAGWIAGDSLSEPIDTAMAWATDANAYVCTVVMPNGAAALTAMKDLYPTYYESVIPTIELQIAKGGYRLGNWLNLIYNASIKRRDLVSGEGIKRAEKKPLPDLMGRDLLPAPRPLSKAKLARAAFGYGCNHEH

SEQ ID NO: 18 - protein, 321 aa, Stenocarpella maydis
WGTLGHATIAYIAQNYVTDEVASWAQGVLSDSSDSYLANIASWADSYRTTTAGAWSAPLHFIDAEDSPPSDCNVDYDRDCGSTGCSVSAIANYTQRVGDGRLSAANVAEALKFLVHFTGDITQPLHDEAYEKGANGVDVTYQGYSDNLHADWDTYLPATLVGGSTLADAKAWATNLTAEINSGIYKSQAAGWIAGDSLSEPIDTAMAWATDANAYVCTVVMPNGAAALTAMKDLYPTYYESVIPTIELQIAKGGYRLGNWLNLIYNASIKRRDLVSGEGIKRAEKKPLPDLMGRDLLPAPRPLSKAKLARAAFGYGCNHEH

SEQ ID NO: 19 - DNA, 1122 bp, Cordyceps cardinalis
  sig_peptide 1..60
  mat_peptide 61..1119
  source 1..1122: mol_type = genomic DNA
  CDS 1..131
  CDS 249..1119
atggtctctttcttccacaccgctgtcgtggcggccactgcctcgcagggcgtgcatgcttggggtgtcttgggccatgccacggtggcttacattgcccagcattacctgactgctgatgctgcgacttggtgggttttcatccaagaccaggaaaaaaagggaaaaaataaagaaaaaaaaaagaaaaaagagggagaaaaaaccagctcttctttgtttggctaacctggaatttcatcacccagggcgcagggtgttctaggcgacacctcagactcgtacctcgccaacatcgcctcctgggccgaccattaccgctccaccaaggcgggcaggtggtccgcgccgctgcacttcatcgacgccgaggacagcccgccgtcgtcgtgcaacgtcgactacgagcgcgactgcggcagcaaggggtgctccgtctccgccatcgccaactacacgcagcgcgtgggcgacgggcgcacgagcgccgcgcacgtcgccgaggcgctcaagttcctcgtgcacttcctcggcgacgtcacgcagccgctgcacgacgaggcgtacgaggtcggcggcaacgacatcaaggtcacctttgcgggctacagcgacaacctgcacgccgactgggacacgtacatgcccgagaagaaggtcggcggcggcaagctgacggacgcgcagtcgtgggcggcggcgctgattggcgagatcgagtcgggccgcttcgagggccaggcggcgggctggattgccggcgatgatgtcggtgacgcgattgcgtcggcgacgcgctgggcgtcggatgcgaatgcgtatgtgtgcagcgtcgtcatgcccgatggcgcggacgcgctgcagcagggcgacttgtacccggcttactacaattcggtgattggcaccatcgagctgcagattgccaagggcggctaccgcctcggaaactggctcaacgccatctatagcggtaagattgcgtcgaagcgcagcgtccatgacaccgcgccgctcccggacctgacgggtcgccatctgctgcctgcgtctcgcccgctgagccgcgcgaagctcgcgagagaggccattggtgagtcgtgctgtacgcacaaccactga

SEQ ID NO: 20 - protein, 334 aa, Cordyceps cardinalis
MVSFFHTAVVAATASQGVHAWGVLGHATVAYIAQHYLTADAATWAQGVLGDTSDSYLANIASWADHYRSTKAGRWSAPLHFIDAEDSPPSSCNVDYERDCGSKGCSVSAIANYTQRVGDGRTSAAHVAEALKFLVHFLGDVTQPLHDEAYEVGGNDIKVTFAGYSDNLHADWDTYMPEKKVGGGKLTDAQSWAAALIGEIESGRFEGQAAGWIAGDDVGDAIASATRWASDANAYVCSVVMPDGADALQQGDLYPAYYNSVIGTIELQIAKGGYRLGNWLNAIYSGKIASKRSVHDTAPLPDLTGRHLLPASRPLSRAKLAREAIGESCCTHNH

SEQ ID NO: 21 - protein, 314 aa, Cordyceps cardinalis
WGVLGHATVAYIAQHYLTADAATWAQGVLGDTSDSYLANIASWADHYRSTKAGRWSAPLHFIDAEDSPPSSCNVDYERDCGSKGCSVSAIANYTQRVGDGRTSAAHVAEALKFLVHFLGDVTQPLHDEAYEVGGNDIKVTFAGYSDNLHADWDTYMPEKKVGGGKLTDAQSWAAALIGEIESGRFEGQAAGWIAGDDVGDAIASATRWASDANAYVCSVVMPDGADALQQGDLYPAYYNSVIGTIELQIAKGGYRLGNWLNAIYSGKIASKRSVHDTAPLPDLTGRHLLPASRPLSRAKLAREAIGESCCTHNH

SEQ ID NO: 22 - DNA, 1175 bp, Phialophora geniculata
  sig_peptide 1..57
  mat_peptide 58..634
  source 1..1175: mol_type = genomic DNA
  CDS 1..70
  CDS 159..634
  CDS 756..1172
atgcgtgtcacaactgtcgcagccctcgtgggcatggccgccgctccggccctggcctggaataccgatggtaagtggctaccggcaaaattcacgcctcggtcaagaggatgaaaactggaggccatgttgaagctgaccatcatactcccctccagtccaccagcaaatcggctacgcgactgaggagttcctcacggaggagacaaaggagatcctcgcccagatcctcgagcccgagcatgagggcagtctaggccgcatctcggcctgggcggactcgcaccgcggcaccgacgagggcaggcacacgacgacctggcactggatcaacccggctgacgagccgccgagcctgtgcaacctctacttcaaccgcgactgcacgggcagcggctgcatcgtcagcgccatcgccaacgaaacgcagatcctcaagggctgcatccgggacgccaagtttggccgtaggcagagcggcaacgggtctgacgcgaccgcctgcgccaacgccgtcaagttcatcacgcactttacgcaggatctggcgcagcccatgcacgtcactggcgttgcgaggggtggcaacgatattcccgtcgtctttgacggcgtcgacacgaacctccacgctgtgagtttgacttccctgatatgcacaacggatttctcttcctctgcaagagagaggggcagagaagaaggcggtcgttctgctggcaaggaatatcacctcgactgacctggcacgctagatctgggacggccgcatcgtctacaccctagccaacacaacccgcttcggtaacgaatcgctcgaccccttcttcgccgacatggtctcccgcatcaaggccgacacctacttcaccccaacggctgagtggctcgaatgcaccagccccggcacgccgctcgcgtgccccattgcctgggcccgtgacaccaaccagtggaactgcgactacgccttcagccagatctacaacggcaccgacctgcgcaccagcggctacgcgcagggtgcctggcccattgccgagatccagatggccaaggctgtgttgcgcaccgccacctggttcaaccgtctcgttgagaattgcttccatgagcgtgaggtcattcttgacttggtgccgagctgggttggtggacctggtggcggtaactaa

SEQ ID NO: 23 - protein, 321 aa, Phialophora geniculata
MRVTTVAALVGMAAAPALAWNTDVHQQIGYATEEFLTEETKEILAQILEPEHEGSLGRISAWADSHRGTDEGRHTTTWHWINPADEPPSLCNLYFNRDCTGSGCIVSAIANETQILKGCIRDAKFGRRQSGNGSDATACANAVKFITHFTQDLAQPMHVTGVARGGNDIPVVFDGVDTNLHAIWDGRIVYTLANTTRFGNESLDPFFADMVSRIKADTYFTPTAEWLECTSPGTPLACPIAWARDTNQWNCDYAFSQIYNGTDLRTSGYAQGAWPIAEIQMAKAVLRTATWFNRLVENCFHEREVILDLVPSWVGGPGGGN

SEQ ID NO: 24 - protein, 302 aa, Phialophora geniculata
WNTDVHQQIGYATEEFLTEETKEILAQILEPEHEGSLGRISAWADSHRGTDEGRHTTTWHWINPADEPPSLCNLYFNRDCTGSGCIVSAIANETQILKGCIRDAKFGRRQSGNGSDATACANAVKFITHFTQDLAQPMHVTGVARGGNDIPVVFDGVDTNLHAIWDGRIVYTLANTTRFGNESLDPFFADMVSRIKADTYFTPTAEWLECTSPGTPLACPIAWARDTNQWNCDYAFSQIYNGTDLRTSGYAQGAWPIAEIQMAKAVLRTATWFNRLVENCFHEREVILDLVPSWVGGPGGGN

SEQ ID NO: 25 - DNA, 1168 bp, Cadophora fastigiata
  sig_peptide 1..60
  mat_peptide 61..1165
  source 1..1168: mol_type = genomic DNA
  CDS 1..339
  CDS 392..448
  CDS 499..510
  CDS 567..992
  CDS 1121..1165
atgagagtcacctacctcattcctctcaccctcctttcctccgttccgagcacactcgcgtggggaactcttgggcatacgacagtagcctacattgcgagcaacttcgtccaacccgagactcgagaccttttccaaaacatcctgcacaatacttctgatgcatacctagctggcatagcgacatgggctgattcgtttcggtacacggctgcggggaagttttctgcgccgtttcatttcattgatgcagaggatagcccgccaaatagttgtgatgtgaagtatgcgagggattgtggggagcaaggttgtgttgttggggctattcagaattatgtgagttgaatttgagcggtcgggtaggaggggcgactgatgatgggtgtagacaagacaactgcttgattctgagctgaatggaggactcagaaatatggctgctaaggtacgccttccaccttcctatatttgtcaaagtactaatgctttgaatagttcgtcgtccacgtaagtatcagtcatattgtagctcaatattaaaaaccagctaataatgcttcaagctcatcggggacatccaccaacctctacatgtcgagaatttagaaaaggggggcaacggcatacaagtcatctttggcggtgcccatgtcaatctccaccacgtctgggatactagtattgcagagaagttagttggaggctacgctcttcctttcgccgaagaatgggctaaaagtctcaccgaggccatcaaggaaaacgagtacaagtctctcgctccatcgtggctggaaggcatcgatttgagtgatccggtcacaacttcgttgggctgggcagaggaaaccaacaaacttgtttgcacggcggtgctccccgctggccgtgagggtgtccaggaccaagagttgagtgggaagtattacgaaaatgcagagcctgttgtgaaactgcaggtagccaaggctgggtatagacttgctagagtaagtgggccggggcaaataattttctattttctctatttttctgttgcaagtcatgtatttgtcctccatgcaaccactcctgagagatttgaagaaaggcatttactgactctgcgatcatctagtggttggatctgattgctcaagcacaggtcaagaagccagagttatga

SEQ ID NO: 26 - protein, 293 aa, Cadophora fastigiata
MRVTYLIPLTLLSSVPSTLAWGTLGHTTVAYIASNFVQPETRDLFQNILHNTSDAYLAGIATWADSFRYTAAGKFSAPFHFIDAEDSPPNSCDVKYARDCGEQGCVVGAIQNYTRQLLDSELNGGLRNMAAKFVVHLIGDIHQPLHVENLEKGGNGIQVIFGGAHVNLHHVWDTSIAEKLVGGYALPFAEEWAKSLTEAIKENEYKSLAPSWLEGIDLSDPVTTSLGWAEETNKLVCTAVLPAGREGVQDQELSGKYYENAEPVVKLQVAKAGYRLARWLDLIAQAQVKKPEL

SEQ ID NO: 27 - protein, 273 aa, Cadophora fastigiata
WGTLGHTTVAYIASNFVQPETRDLFQNILHNTSDAYLAGIATWADSFRYTAAGKFSAPFHFIDAEDSPPNSCDVKYARDCGEQGCVVGAIQNYTRQLLDSELNGGLRNMAAKFVVHLIGDIHQPLHVENLEKGGNGIQVIFGGAHVNLHHVWDTSIAEKLVGGYALPFAEEWAKSLTEAIKENEYKSLAPSWLEGIDLSDPVTTSLGWAEETNKLVCTAVLPAGREGVQDQELSGKYYENAEPVVKLQVAKAGYRLARWLDLIAQAQVKKPEL

SEQ ID NO: 28 - DNA, 813 bp, Unidentified
  misc_feature 1..813: note = Microbial metagenome enrichment
  sig_peptide 1..90
  mat_peptide 91..810
  source 1..813: mol_type = genomic DNA
  CDS 1..810
atgcctgaagttttttcttccccggcctcagtaatccgccgaatggccctcttactgatagccttttctttaagcgttaccgccttctgttggggtcttaccggacaccgggtaattggcgaaatagctgaaaaccatttatccagaaaagcaaaaaaggagctgaaaaaactgattggccgccaaacgctcgcctggtggtctaactggcctgattttatcaagtcagacactacctggaatcatgcttcgccctggcattatgtaaatgtgcagcctcacctctctaaagaagaatttgtggaggcattaaaaaaactgccgggcaaaaacctatatacacagatccaggaaacgatgagccagcttaaagaccggcaattgtcgctggagcaacggcaaatagccctccggtttctggtacacctggtaggtgatctgcatcagcccctgcatgtgggtcatgccgatgaccagggtggaaataaaattgtagtatactggtttgatcggaagaccaaccttcattctgtgtgggatacctggcttattgatgaacagaaatacagctattccgaatatgcccgcctgcttgatatcactcccaaagaacaggtagaggcctggcaatcttcgtcactcgaggattggttttatgaatcttaccagctggcagaaaaaatctatgcttcggtacagcccgaagacaaacttagttatcgttacaattacctgtttcagtcgttcatggatgagcaactggtaaaaggtggtgtacgcctggctgccctcctcaataacagcctcgaataa

SEQ ID NO: 29 - protein, 270 aa, Unidentified
  REGION 1..270: note = Synthetic Construct
MPEVFSSPASVIRRMALLLIAFSLSVTAFCWGLTGHRVIGEIAENHLSRKAKKELKKLIGRQTLAWWSNWPDFIKSDTTWNHASPWHYVNVQPHLSKEEFVEALKKLPGKNLYTQIQETMSQLKDRQLSLEQRQIALRFLVHLVGDLHQPLHVGHADDQGGNKIVVYWFDRKTNLHSVWDTWLIDEQKYSYSEYARLLDITPKEQVEAWQSSSLEDWFYESYQLAEKIYASVQPEDKLSYRYNYLFQSFMDEQLVKGGVRLAALLNNSLE

SEQ ID NO: 30 - protein, 240 aa, Unidentified
  REGION 1..240: note = Microbial enrichment
WGLTGHRVIGEIAENHLSRKAKKELKKLIGRQTLAWWSNWPDFIKSDTTWNHASPWHYVNVQPHLSKEEFVEALKKLPGKNLYTQIQETMSQLKDRQLSLEQRQIALRFLVHLVGDLHQPLHVGHADDQGGNKIVVYWFDRKTNLHSVWDTWLIDEQKYSYSEYARLLDITPKEQVEAWQSSSLEDWFYESYQLAEKIYASVQPEDKLSYRYNYLFQSFMDEQLVKGGVRLAALLNNSLE

SEQ ID NO: 31 - DNA, 861 bp, Lysobacter enzymogenes
  sig_peptide 1..102
  mat_peptide 103..858
  source 1..861: mol_type = genomic DNA
  CDS 1..858
atgatcgaaccgcgcacccgcccccgcttcgcccgccgcctgctcgacgccgccgtcgtgctcgccgtcgtgtccgccgccctgccgctgccggccctggcctggggccgccaaggccatcgactggtcgccgagctggcctgggacgaaatgtccccagccgcgcgcgccgaagcgcaaaagctgctcgccggcgaacccgacccgaccctgcccggcatcgccaactgggccgacctgctgcgcgacgagaagccggaactgggcaagcgcagcgccaagtggcattacatcgacctgcccgaagacggcgactgccagtacgacgccgcccgcgattgccccgacggcaactgtgcggtcgccgcgatcctcgcccagaccgcgatcctggccgaccgcagcaagcccaaggccgaacgcctggaggcgctgaaattcgtcgtccacctggtcggcgacgtgcaccagccgctgcacgtcggctacgcccgcgacaagggcggcaatagcttccagctcgccatccccggcgagcgtcccggcggctacggcggcaacctgcattcgctgtgggacagcggcatgcccaacatgagcaagctcgatgacgataagtacctggccaaactcaaggacatccaggtgcccgcggtcagcgccctggcgctgccgccgccggccgccgactgggccgaggaatcgtgcaaactgatgcggcaaccgggtttctacccgcccacgcacaagctggcccccgactacgtcgccacctggcggccgctggccgaaacccaactgcgcctgggcggcgaacacctggccaagctgctcgacgccgccctggcgccgggccgctga

SEQ ID NO: 32 - protein, 286 aa, Lysobacter enzymogenes
MIEPRTRPRFARRLLDAAVVLAVVSAALPLPALAWGRQGHRLVAELAWDEMSPAARAEAQKLLAGEPDPTLPGIANWADLLRDEKPELGKRSAKWHYIDLPEDGDCQYDAARDCPDGNCAVAAILAQTAILADRSKPKAERLEALKFVVHLVGDVHQPLHVGYARDKGGNSFQLAIPGERPGGYGGNLHSLWDSGMPNMSKLDDDKYLAKLKDIQVPAVSALALPPPAADWAEESCKLMRQPGFYPPTHKLAPDYVATWRPLAETQLRLGGEHLAKLLDAALAPGR

SEQ ID NO: 33 - protein, 252 aa, Lysobacter enzymogenes
WGRQGHRLVAELAWDEMSPAARAEAQKLLAGEPDPTLPGIANWADLLRDEKPELGKRSAKWHYIDLPEDGDCQYDAARDCPDGNCAVAAILAQTAILADRSKPKAERLEALKFVVHLVGDVHQPLHVGYARDKGGNSFQLAIPGERPGGYGGNLHSLWDSGMPNMSKLDDDKYLAKLKDIQVPAVSALALPPPAADWAEESCKLMRQPGFYPPTHKLAPDYVATWRPLAETQLRLGGEHLAKLLDAALAPGR

SEQ ID NO: 34 - DNA, 855 bp, Pseudoalteromonas nigrifaciens
  sig_peptide 1..78
  mat_peptide 79..852
  source 1..855: mol_type = genomic DNA
  CDS 1..852
atgtacaaaaatgtaaaatattcgctttctttaacacttttaattagtgcaatatttgcaagcacagattgtaatgcttggggccaaaacgggcatcgtgtagtgggtaaaattgcagagtcgcacataagcgaaactactaagactgcacttatcccctatctcaacggcgaatctttagctcagatttcaacatggcctgacgaaatgcgatctgcaccaggtgacttttggcaaaaaaaatcatctcgttggcattacattaatgctgatgacaacgcgacctttagttttgaccacgaccacactaaacacaaagaatcagtaacaaacatattagaaggcattcactactctatccgcgtgttgaaagatgataaatcaagccttgcggccaaacagtttagcttgcgttttttggtgcatttagtgggtgacagccatcaaccatttcatgcaggaagagcagaagatagaggcggcaatcgcgttaaggtttctttttttaaccaacaaaccaatcttcatagcttatgggataccaagttagtagaaaacgaaaacttatcttttactgaatacgctcagtttataaataccaacaacagcgagctaattagtgaatatttacaaagtacaccaacaagctggcttgaagagtcgcacaacttagccctaaaaatatatgaatcaacagaagaacagattagctacgactatatctacaacaacacccctattgtgaaaacacgcttgcaacaagcgggaattcgcttggctggattgttaaatacattatttgatcctgctaacacgcaatcaaaaatggccttaacacagcaaacacaaaaataa

SEQ ID NO: 35 - protein, 284 aa, Pseudoalteromonas nigrifaciens
MYKNVKYSLSLTLLISAIFASTDCNAWGQNGHRVVGKIAESHISETTKTALIPYLNGESLAQISTWPDEMRSAPGDFWQKKSSRWHYINADDNATFSFDHDHTKHKESVTNILEGIHYSIRVLKDDKSSLAAKQFSLRFLVHLVGDSHQPFHAGRAEDRGGNRVKVSFFNQQTNLHSLWDTKLVENENLSFTEYAQFINTNNSELISEYLQSTPTSWLEESHNLALKIYESTEEQISYDYIYNNTPIVKTRLQQAGIRLAGLLNTLFDPANTQSKMALTQQTQK

SEQ ID NO: 36 - protein, 258 aa, Pseudoalteromonas nigrifaciens
WGQNGHRVVGKIAESHISETTKTALIPYLNGESLAQISTWPDEMRSAPGDFWQKKSSRWHYINADDNATFSFDHDHTKHKESVTNILEGIHYSIRVLKDDKSSLAAKQFSLRFLVHLVGDSHQPFHAGRAEDRGGNRVKVSFFNQQTNLHSLWDTKLVENENLSFTEYAQFINTNNSELISEYLQSTPTSWLEESHNLALKIYESTEEQISYDYIYNNTPIVKTRLQQAGIRLAGLLNTLFDPANTQSKMALTQQTQK

SEQ ID NO: 37 - DNA, 825 bp, Vibrio sp.
  sig_peptide 1..69
  mat_peptide 70..822
  source 1..825: mol_type = genomic DNA
  CDS 1..822
atggcagccatgaacagaatcctgctgctcgccgccctgctgttgctgatcgccgcccccgcccacgcctggggcccgcgcggccaccgcctggtgggacatgtcgccgaatccgaactcacgcccgccgcccgcatccaggtcgatgcgctgctggccggggtggaagatccgcatgaccgctcgctggcgggcattgccacctgggctgacgagcttcgcggctccgacccggagctgggccggaaaagcgcgcgctggcacttcgtgaacatcggcgagaacggctgccgctaccagcgcgagcgcgactgcccggacggcgccaactgcgtggtcgaggcgatcaacgaccaggccgcgatcctcggtgaccgcacgcgcagcgaggccgaacggctgcaggcgctgaagttcgtggtccacttcgtcggcgacgtacaccagccgatgcacgccggctacgcccacgaccgcggcggcaacacattccaggtcaacctgcgcgatggcacccgcgacgggcgcggcaccaacctgcacgcgctgtgggacagcggcctgttctacggtcttcgcgagtcggaagagcggcatctggccgccttgcgcgagctgccggacgacgcgccggctggcggcagcccggccacctgggccgaagagtcctgcgccgtggcgctgcaggacggcgtatatccgccacacgccgtcatcggacaggactacgtggaccagtggcggccggtcgccgagcagcgcatcgttgccggtggcaagcgcctggcgcggctgctcaaccagatactcgaccccgtccgctga

SEQ ID NO: 38 - protein, 274 aa, Vibrio sp.
MAAMNRILLLAALLLLIAAPAHAWGPRGHRLVGHVAESELTPAARIQVDALLAGVEDPHDRSLAGIATWADELRGSDPELGRKSARWHFVNIGENGCRYQRERDCPDGANCVVEAINDQAAILGDRTRSEAERLQALKFVVHFVGDVHQPMHAGYAHDRGGNTFQVNLRDGTRDGRGTNLHALWDSGLFYGLRESEERHLAALRELPDDAPAGGSPATWAEESCAVALQDGVYPPHAVIGQDYVDQWRPVAEQRIVAGGKRLARLLNQILDPVR

SEQ ID NO: 39 - protein, 251 aa, Vibrio sp.
WGPRGHRLVGHVAESELTPAARIQVDALLAGVEDPHDRSLAGIATWADELRGSDPELGRKSARWHFVNIGENGCRYQRERDCPDGANCVVEAINDQAAILGDRTRSEAERLQALKFVVHFVGDVHQPMHAGYAHDRGGNTFQVNLRDGTRDGRGTNLHALWDSGLFYGLRESEERHLAALRELPDDAPAGGSPATWAEESCAVALQDGVYPPHAVIGQDYVDQWRPVAEQRIVAGGKRLARLLNQILDPVR

SEQ ID NO: 40 - DNA, 1038 bp, Janthinobacterium agaricidamnosum
  sig_peptide 1..63
  mat_peptide 64..1035
  source 1..1038: mol_type = genomic DNA
  CDS 1..1035
atgaagaagctcgtatgtattctggcgttgagcagcgcctttgtttcgtttaacgcccacgcatggggcaacgatggccaccgcgcggtgggcgcgatcgccgatcaactgatcaaaggcagcaacgccgaacagcaggtgcaagccttgctgctgccgggcgaaagcctggccggcatcgcctcgtgggcggactgcgtgaagggcacttactgcggtccgcaaacgccggagatggtggcttacaccacggccaatccgaagcacagcgaatatcactacaccgatgtgccgttccagctgtcgcactatgaagacggcggcgttggcacccacggtcacgatatcgtgcagacgctgaagcagtgcatcgcggtgctgcaagggaaaggcgatgcgaccaccaatccacacaacttcacgccgcgccaggctttgctgatgctgacgcacctgactggcgacatcgcgcagccgctgcatgtgggcgaaggctatgtcggcaagagcggcggctttgtgctgccgacgcagaagcagttggacaacaaggaagccttcccgtccaccggtggcaacaacctgcaactggacgacgccaagttgaccactagcagcgcgcagctgattccggccgatgacaaaccggccaagccgcaggccacgcgcgccttccactcgtactgggataccacggtggtcaactacgccttccgccgcatcggcgcgcgcacgccggagcagtttgcgcagatggtgattgctgatggtccggtggtggcgccgagcagcggcgacccggtaacgtggccgtaccagtgggccgaccaggcgctggtggtggcgaagctcgcctataccgacgtggtgcccggcccgatggggcagcagaccagcaagagcagcggtgaggtgtacaacgtgtggccgctggcgattccggacaactatccggtgccatcgtcggctgcggccaagacgcagctgatccagggcggttatcacctggcggccgtgctgaaggccatctggccgtaa

SEQ ID NO: 41 - protein, 345 aa, Janthinobacterium agaricidamnosum
MKKLVCILALSSAFVSFNAHAWGNDGHRAVGAIADQLIKGSNAEQQVQALLLPGESLAGIASWADCVKGTYCGPQTPEMVAYTTANPKHSEYHYTDVPFQLSHYEDGGVGTHGHDIVQTLKQCIAVLQGKGDATTNPHNFTPRQALLMLTHLTGDIAQPLHVGEGYVGKSGGFVLPTQKQLDNKEAFPSTGGNNLQLDDAKLTTSSAQLIPADDKPAKPQATRAFHSYWDTTVVNYAFRRIGARTPEQFAQMVIADGPVVAPSSGDPVTWPYQWADQALVVAKLAYTDVVPGPMGQQTSKSSGEVYNVWPLAIPDNYPVPSSAAAKTQLIQGGYHLAAVLKAIWP

SEQ ID NO: 42 - protein, 324 aa, Janthinobacterium agaricidamnosum
WGNDGHRAVGAIADQLIKGSNAEQQVQALLLPGESLAGIASWADCVKGTYCGPQTPEMVAYTTANPKHSEYHYTDVPFQLSHYEDGGVGTHGHDIVQTLKQCIAVLQGKGDATTNPHNFTPRQALLMLTHLTGDIAQPLHVGEGYVGKSGGFVLPTQKQLDNKEAFPSTGGNNLQLDDAKLTTSSAQLIPADDKPAKPQATRAFHSYWDTTVVNYAFRRIGARTPEQFAQMVIADGPVVAPSSGDPVTWPYQWADQALVVAKLAYTDVVPGPMGQQTSKSSGEVYNVWPLAIPDNYPVPSSAAAKTQLIQGGYHLAAVLKAIWP

SEQ ID NO: 43 - DNA, 1062 bp, Massilia aerilata
  sig_peptide 1..63
  mat_peptide 64..1059
  source 1..1062: mol_type = genomic DNA
  CDS 1..1059
atgaaaaaactcgcttgtgtaatcgccctggccggcgccttcatggcgcaggacgctttcgcctggggccacgacggccatgccgccgtgggcgccatcgccgacaaactgatcaagggcaccgacgccgagaagcaggtcaaggccctgctgctgccgggcgagtcgctcgaatcgatcgccaactggccggactgcgtgaagggcaattactgcggtccgcagacccaggaaatgatcgattacgtgaacgccaacccgctgcacagcgaataccactacaccgacgtgccgttccagctcgagcactaccatgacggcgaagtcggcaccaccgacgtcgacatcgtgcagacgctgaaggaagcgatcgccgtcctgcagggcaaggacacgcctcagaccaatccgcatcacttcaccaagcgccaggccctgatcctgatcacccatctggtgggcgacatccaccagccgctgcacgtgggcgcggcctatgtcgacaaggacggcaagttcatcgtgccgaagaccaaggccgagatcgacgagaccgtggtcttcgattcgcgcggcggcaacaacttcctgatgaacgacgagaagatcgaacagttcgcggccaaggcggccgacgtgatcccgccggccgagcaggccgaaggcaagccgggcgtgccgaaggcgctaaccaagcctttccactcgtactgggacacgaccgtggtcaactacgccttccgccgcgtgcgcaccaagaccccggagcagttcgcgcaggtgaccatcgacagccatccggacgtcgtcaagaacagcggcgacccggtgacctggccttaccagtgggccgacgacgccctggtcgtggccaagcaggccttcgacggtgtccacgtcggccagatcaccaagcagaccagcaagaaaggcgacacctactacacctggagcctggaagtgccgcagaactacccggtgccgagctcggcgatcgccaaggcgcagctggccaagggtggttatcacctggcggaggtgctggaagcgatctggccgtaa

SEQ ID NO: 44 - protein, 353 aa, Massilia aerilata
MKKLACVIALAGAFMAQDAFAWGHDGHAAVGAIADKLIKGTDAEKQVKALLLPGESLESIANWPDCVKGNYCGPQTQEMIDYVNANPLHSEYHYTDVPFQLEHYHDGEVGTTDVDIVQTLKEAIAVLQGKDTPQTNPHHFTKRQALILITHLVGDIHQPLHVGAAYVDKDGKFIVPKTKAEIDETVVFDSRGGNNFLMNDEKIEQFAAKAADVIPPAEQAEGKPGVPKALTKPFHSYWDTTVVNYAFRRVRTKTPEQFAQVTIDSHPDVVKNSGDPVTWPYQWADDALVVAKQAFDGVHVGQITKQTSKKGDTYYTWSLEVPQNYPVPSSAIAKAQLAKGGYHLAEVLEAIWP

SEQ ID NO: 45 - protein, 332 aa, Massilia aerilata
WGHDGHAAVGAIADKLIKGTDAEKQVKALLLPGESLESIANWPDCVKGNYCGPQTQEMIDYVNANPLHSEYHYTDVPFQLEHYHDGEVGTTDVDIVQTLKEAIAVLQGKDTPQTNPHHFTKRQALILITHLVGDIHQPLHVGAAYVDKDGKFIVPKTKAEIDETVVFDSRGGNNFLMNDEKIEQFAAKAADVIPPAEQAEGKPGVPKALTKPFHSYWDTTVVNYAFRRVRTKTPEQFAQVTIDSHPDVVKNSGDPVTWPYQWADDALVVAKQAFDGVHVGQITKQTSKKGDTYYTWSLEVPQNYPVPSSAIAKAQLAKGGYHLAEVLEAIWP

SEQ ID NO: 46 - DNA, 963 bp, Aspergillus oryzae
  sig_peptide 1..60
  mat_peptide 61..960
  source 1..963: mol_type = genomic DNA
  CDS 1..339
  CDS 389..454
  CDS 505..960
atgccgcgcttactccccatttcggcggctactctggcgctggcccaactcacttatggttggggcaacttaggtcacgagacggttgcctacattgcccagagcttcgttgcttcgtcaaccgaatccttttgtcagaatattctgggcgatgactccacctcctacttggccaacgtcgccacatgggccaatacgtacaagtacaccgacgccggagaattctccaagccctaccattttattgatgcgcaagacaacccgccgcagagctgtggagtagattacgatcgggattgtggtagcgctgggtgcagtatcagcgcgatccagaactacgtgagttactttagagtttataacgatataggatgctcatcctacttagaccaatattctcttggaatctcccaatggctcggaggcgttgaatgccctgaaattcgtggttcatgttcgtcacgttgcttgaccggtttaatccgatttcctaacatgtcgcagatcatcggagatattcaccagcccttacacgacgagaatctcgaggccggtggtaacggtattgatgtgacgtatgatggagaaaccaccaatctccatcatatctgggacaccaacatgcccgaggaagccgcaggtggctacagtctgtcggtcgcaaagacctatgctgaccttcttactgagcgcatcaaaaccggtacctactcgtctaagaaggactcatggacagacggaattgatatcaaagaccctgtgtcgacctcaatgatctgggcggccgacgccaatacctacgtctgcagtacggttttggatgacggactggcatatattaactccacagacctgtctggcgagtactacgacaagtcccagccagtctttgaggaactcatcgcgaaggctgggtatcggctggcggcttggttggatctgattgcgagtcagccctcttga

SEQ ID NO: 47 - protein, 287 aa, Aspergillus oryzae
MPRLLPISAATLALAQLTYGWGNLGHETVAYIAQSFVASSTESFCQNILGDDSTSYLANVATWANTYKYTDAGEFSKPYHFIDAQDNPPQSCGVDYDRDCGSAGCSISAIQNYTNILLESPNGSEALNALKFVVHIIGDIHQPLHDENLEAGGNGIDVTYDGETTNLHHIWDTNMPEEAAGGYSLSVAKTYADLLTERIKTGTYSSKKDSWTDGIDIKDPVSTSMIWAADANTYVCSTVLDDGLAYINSTDLSGEYYDKSQPVFEELIAKAGYRLAAWLDLIASQPS

SEQ ID NO: 48 - protein, 267 aa, Aspergillus oryzae
WGNLGHETVAYIAQSFVASSTESFCQNILGDDSTSYLANVATWANTYKYTDAGEFSKPYHFIDAQDNPPQSCGVDYDRDCGSAGCSISAIQNYTNILLESPNGSEALNALKFVVHIIGDIHQPLHDENLEAGGNGIDVTYDGETTNLHHIWDTNMPEEAAGGYSLSVAKTYADLLTERIKTGTYSSKKDSWTDGIDIKDPVSTSMIWAADANTYVCSTVLDDGLAYINSTDLSGEYYDKSQPVFEELIAKAGYRLAAWLDLIASQPS

SEQ ID NO: 49 - DNA, 1102 bp, Penicillium atramentosum
  sig_peptide 1..57
  mat_peptide 58..1099
  source 1..1102: mol_type = genomic DNA
  CDS 1..128
  CDS 199..1099
atggtgtctctatctatcgctctcgtgacactcggggtggctcatggggcaaatgcatggggtgtattgggccatgcggctgttgcctatgtcgcacaaagttatgtttgtcctgaagccgcatcatggtaagtgcctccaaagagcttgaactcagcaagagtatctttctttcaatgctaaaacatttttttcaagggcacaaaaagttctcaatgattcctctagctcatacctagcaaacattgcttcttgggctgatcagtatcgcctaacagatgctggaaaatggtcagccccattgcattacattgatgccttggatgaccccccaaaaagttgcaacgtcgactacgagcgcgactgttctgatgaaggctgttcaatctccgccattgccaattacactcaacgtgcaggcgaccgccgacttgacaatgttcataccgcagaagctctcagatttctggtccactttctcggcgatctcacgcagccactgcatgatgaggcctacgagattggtggcaatggcatcaacgtcacttttgacggatataatgacaaccttcacgcagattgggatacatacatccccgtgaagctggtcggcggtagctcactcgcggatgcgcaatcatgggccactagcctggttaaggagatcacctccggcagcttcaaatacaaatctgcgaattggattagaggcgacaatatcgatgatgttatcaccacggccacgagatgggcgacagatgccaacgcctacatctgtaccgttgttatgccagatggggctgctgccttgcaaactggcgacctctatccaacttactacgagtctgccatcggcactatcgagttgcagattgctaaaggaggctacagactgggcaactggatcaacttgatctatagaaataagatcgctaaacatcgccccggcaaacccgttaaagagcaccatggaccaccccctcactcaaaccatggggacgatattcgtcctcatcctcgtggaccgagtcgcgcaaatttggccagggcggccatgggaggatcctgctgtagctctgggggacgtgaacacgaacactga

SEQ ID NO: 50 - protein, 343 aa, Penicillium atramentosum
MVSLSIALVTLGVAHGANAWGVLGHAAVAYVAQSYVCPEAASWAQKVLNDSSSSYLANIASWADQYRLTDAGKWSAPLHYIDALDDPPKSCNVDYERDCSDEGCSISAIANYTQRAGDRRLDNVHTAEALRFLVHFLGDLTQPLHDEAYEIGGNGINVTFDGYNDNLHADWDTYIPVKLVGGSSLADAQSWATSLVKEITSGSFKYKSANWIRGDNIDDVITTATRWATDANAYICTVVMPDGAAALQTGDLYPTYYESAIGTIELQIAKGGYRLGNWINLIYRNKIAKHRPGKPVKEHHGPPPHSNHGDDIRPHPRGPSRANLARAAMGGSCCSSGGREHEH

SEQ ID NO: 51 - protein, 324 aa, Penicillium atramentosum
WGVLGHAAVAYVAQSYVCPEAASWAQKVLNDSSSSYLANIASWADQYRLTDAGKWSAPLHYIDALDDPPKSCNVDYERDCSDEGCSISAIANYTQRAGDRRLDNVHTAEALRFLVHFLGDLTQPLHDEAYEIGGNGINVTFDGYNDNLHADWDTYIPVKLVGGSSLADAQSWATSLVKEITSGSFKYKSANWIRGDNIDDVITTATRWATDANAYICTVVMPDGAAALQTGDLYPTYYESAIGTIELQIAKGGYRLGNWINLIYRNKIAKHRPGKPVKEHHGPPPHSNHGDDIRPHPRGPSRANLARAAMGGSCCSSGGREHEH

SEQ ID NO: 52 - DNA, 955 bp, Penicillium emersonii
  sig_peptide 1..57
  mat_peptide 58..952
  source 1..955: mol_type = genomic DNA
  CDS 1..336
  CDS 381..449
  CDS 500..952
atgccctcccgcgcctcccttctcgccctcctggccgccctcccccgcagctacgcctggggcaatcttggccacgagacgattgcctacatcgcgcagaacttcatcgcctcgtcgacggcgtcctactgccaggacgtcctgggcgatacgtcctcctcgtatctggccagcgtagccacctgggcggactcgtaccgctacacgtccgccggccgctggtccgagccgctgcactttatcgatgcgcatgacgatcccccgacgagctgcggcgtcgactatgagagggactgcggcgataagggatgcgttgtgagcgcgatcaataactacgtaagctctcgatctacgttaggaggttctggctgacaattcagacgacgcaattactggacaaatcgacatcgagttccgaagcgttggacgcgatgaaattcatcatccacgttagtcgcacgtacacacccttttcgtcgtgtttagctaacgaagccagttcgtcggtgacatccaccagccccttcacgacgagaacctcgacgtgggcggcaacgacatcgacgtgaccttcaacggcacgaaaacgaacctgcaccacatctgggacacgaacatgctggaagccgacgcgggggggtcatcactgtcgacggccaagagcttcgcggcgacgctgaccacgcgcctgcagagcggcgagtacgcgtcgcagaaggcgtcgtggatctcgggcatgaatatcagcgatccggtggccacggcgatgggctgggcaagcgatgccaacgggtacgtctgcagcacggtgctgaagccgggactgtcgtatatcaaggacaacgatctgtcggggaagtactatgccgactgcaagccggtgtttgaggagctgcttgcccgggcaggataccggttggcggcgtggttggacttgattgcgaagcagcagtag

SEQ ID NO: 53 - protein, 286 aa, Penicillium emersonii
MPSRASLLALLAALPRSYAWGNLGHETIAYIAQNFIASSTASYCQDVLGDTSSSYLASVATWADSYRYTSAGRWSEPLHFIDAHDDPPTSCGVDYERDCGDKGCVVSAINNYTTQLLDKSTSSSEALDAMKFIIHFVGDIHQPLHDENLDVGGNDIDVTFNGTKTNLHHIWDTNMLEADAGGSSLSTAKSFAATLTTRLQSGEYASQKASWISGMNISDPVATAMGWASDANGYVCSTVLKPGLSYIKDNDLSGKYYADCKPVFEELLARAGYRLAAWLDLIAKQQ

SEQ ID NO: 54 - protein, 267 aa, Penicillium emersonii
WGNLGHETIAYIAQNFIASSTASYCQDVLGDTSSSYLASVATWADSYRYTSAGRWSEPLHFIDAHDDPPTSCGVDYERDCGDKGCVVSAINNYTTQLLDKSTSSSEALDAMKFIIHFVGDIHQPLHDENLDVGGNDIDVTFNGTKTNLHHIWDTNMLEADAGGSSLSTAKSFAATLTTRLQSGEYASQKASWISGMNISDPVATAMGWASDANGYVCSTVLKPGLSYIKDNDLSGKYYADCKPVFEELLARAGYRLAAWLDLIAKQQ

SEQ ID NO: 55 - DNA, 1086 bp, Ostropa Barbara
  sig_peptide 1..57
  mat_peptide 58..1083
  source 1..1086: mol_type = genomic DNA
  CDS 1..336
  CDS 403..459
  CDS 507..518
  CDS 569..984
  CDS 1035..1083
atgaaatacacaaccctcaccctcaccttcctctcctccctccctttctcctatgcctggggttccctcggccatgaaaccgtcgcttacatcgcctccaacttcgtcgcgcccccaacgcgcaccttcttccagtccatcctccacaacaccagcgattcctatctagccagcgtggccacctgggctgattccttcagatacacagctgctgggcgcttctcggcgccgttccattttattgatgcagaggatagcccgccgcatacttgtggtgtgaagtattcgcgggactgtggggagcaggggtgtgtggtgggggctatttctaattatgtgagttattcatgattttgtgatacggtactcctggttttgagagaggatggctaatggaagtagacgagtcagttgctggatccggagttggatgcttggaagaggacgatggctgccaaggtatgatttccccggtattttggatatcggatactaacgagaagtagtttattgttcatgtaaggaatcgaaagtccttttctgaaagcagtcattaacagccagaaagttcttaggagatattcatcaaccactccacgatgagaatctcgaccggggtgggaactctattcccgtgctcttcgactccgtttccaccaatttacatcatctttgggatagtaacatacccgaaaaactcatcggaggctactcactgcctgacgccgagcgctgggctactgcgctcactgcggctatcaagaccggcatctataagccattagcagcggagtggctacatggcatggatttgaaagatccggtaagtacgagcttgaagtgggcggaggaagcgaatgcttttgtgtgttctactgtgttgccggagggaaaagagggtgtaatggggaaggaactgaatgggagttactatgagaatgcggtgcctgtgattcagttgcaaattgcgagggcggggtataggtgagttgagattcttgttcttggaaagaagaaactaatcgtttgagtagactggctcgttggttggacttgattgcagctgacggtaaaacggaattgtaa

SEQ ID NO: 56 - protein, 290 aa, Ostropa Barbara
MKYTTLTLTFLSSLPFSYAWGSLGHETVAYIASNFVAPPTRTFFQSILHNTSDSYLASVATWADSFRYTAAGRFSAPFHFIDAEDSPPHTCGVKYSRDCGEQGCVVGAISNYTSQLLDPELDAWKRTMAAKFIVHFLGDIHQPLHDENLDRGGNSIPVLFDSVSTNLHHLWDSNIPEKLIGGYSLPDAERWATALTAAIKTGIYKPLAAEWLHGMDLKDPVSTSLKWAEEANAFVCSTVLPEGKEGVMGKELNGSYYENAVPVIQLQIARAGYRLARWLDLIAADGKTEL

SEQ ID NO: 57 - protein, 271 aa, Ostropa Barbara
WGSLGHETVAYIASNFVAPPTRTFFQSILHNTSDSYLASVATWADSFRYTAAGRFSAPFHFIDAEDSPPHTCGVKYSRDCGEQGCVVGAISNYTSQLLDPELDAWKRTMAAKFIVHFLGDIHQPLHDENLDRGGNSIPVLFDSVSTNLHHLWDSNIPEKLIGGYSLPDAERWATALTAAIKTGIYKPLAAEWLHGMDLKDPVSTSLKWAEEANAFVCSTVLPEGKEGVMGKELNGSYYENAVPVIQLQIARAGYRLARWLDLIAADGKTEL

SEQ ID NO: 58 - DNA, 1047 bp, Pyrenochaetopsis sp.
  sig_peptide 1..54
  mat_peptide 55..1044
  source 1..1047: mol_type = genomic DNA
  CDS 1..67
  CDS 132..598
  CDS 649..1044
atgcttcccagacactcgctcctgagcgccatcctggtagcttcagccacagcatggaatatcgacggtacgcgtactcactccgaccgcagccctccaccacagtagctcgcaacactaaccgcatccagtgcacaaccaaatcggcttcatggccgaaacattcctaacgcctcaaacaaccgctattctcgccgagctgctcgaaccccagtacaacagctccataggccgcgccgcagcgtgggcagacgcctacgcgcatacgcaggagggccgcttctcgtaccaatggcactggattgatacgcatgatagcgcgcccgagtactgcagcttaaattatacagcggactgtgcaaaaggcggatgtgttgttagtgcgattgcgaatcagacgggcatactaagagagtgcatagaggatgtgaaggttggggttgtgggcggtgggagtaacttgacgtgttcgtatgcgctgaagtgggttacgcattttctgggggatgtacatcagccgctgcatgctaatgggagagctgtgggggggaatacgtataaagtcacgtttggcggcgtggctacgcagttgcatgctgtaaatacgcccaaatcccgctctctcgtgaaagtactaacgtgcattaggtctgggacggatatatcccctacttcgcagccagcgtcagcaaaccattttcaaaccagtccattgatcctttcttcactggtctggtgtctcggatccgcaatgatgggttcttctctgcgccgtatatgtggttggcttgcgttgatccttctacggcagcagaatgtgctacgaggtgggcaaaggagagtaataagtgggactgcgattatgtctggagccgcgtacggaatgacactgatctgggaactaatgggtatgcggtcggtgctgtacctattgtggaattgcaaatcagcaaagctgcgctcagattgggtacttggctcaacaggcttgtcgaaggggacaagtatgagtatgcttcacgcaaccaagaggtcattgagctttaa

SEQ ID NO: 59 - protein, 310 aa, Pyrenochaetopsis sp.
MLPRHSLLSAILVASATAWNIDVHNQIGFMAETFLTPQTTAILAELLEPQYNSSIGRAAAWADAYAHTQEGRFSYQWHWIDTHDSAPEYCSLNYTADCAKGGCVVSAIANQTGILRECIEDVKVGVVGGGSNLTCSYALKWVTHFLGDVHQPLHANGRAVGGNTYKVTFGGVATQLHAVWDGYIPYFAASVSKPFSNQSIDPFFTGLVSRIRNDGFFSAPYMWLACVDPSTAAECATRWAKESNKWDCDYVWSRVRNDTDLGTNGYAVGAVPIVELQISKAALRLGTWLNRLVEGDKYEYASRNQEVIEL

SEQ ID NO: 60 - protein, 292 aa, Pyrenochaetopsis sp.
WNIDVHNQIGFMAETFLTPQTTAILAELLEPQYNSSIGRAAAWADAYAHTQEGRFSYQWHWIDTHDSAPEYCSLNYTADCAKGGCVVSAIANQTGILRECIEDVKVGVVGGGSNLTCSYALKWVTHFLGDVHQPLHANGRAVGGNTYKVTFGGVATQLHAVWDGYIPYFAASVSKPFSNQSIDPFFTGLVSRIRNDGFFSAPYMWLACVDPSTAAECATRWAKESNKWDCDYVWSRVRNDTDLGTNGYAVGAVPIVELQISKAALRLGTWLNRLVEGDKYEYASRNQEVIEL

SEQ ID NO: 61 - DNA, 1448 bp, Lachnellula sp.
  sig_peptide 1..63
  mat_peptide 64..1445
  source 1..1448: mol_type = genomic DNA
  CDS 1..112
  CDS 190..269
  CDS 322..471
  CDS 523..579
  CDS 626..637
  CDS 831..935
  CDS 1028..1338
  CDS 1400..1445
atgaagactcaagctgtagcttccattttcttcctcgccacctctcttccctccgctttaggctggggcactcttggtcatcagacagtagcttatatcgctacaaactttggttaacagcctagctttcaacataaccgctgtttaaaatcgattgtgatagcatcatgtgctgatatatcgaaatagtttcaagtgccacgaaaaccaaattccaaagcatcttggacgacacaagcaccgactatcttgcctcagttgcaacatgggtaatagatctgctaatccactttctcacttggtgctgatgtcttcttgcaggctgatacttatcgctacaccgatgccgggacctggacggccccgtttcacttcattgatgccaatgacgatcccccatcaagctgcagcgtagattattcccgtgattgtggaacgggaggttgtgtgattagcgctatcaataattatgtacgctgcaaggaattgatgatactggatttggtctaatgagtaaaacagacctcgcgtattcagagtaccaccttgagcgacgcagaggtgaagactgctgcaaaggtaaggacgagcaaaccctggttgcttgaatactaacagcctccagttcatcgtccatgtaagttgtgaatcctttgagcagaagttcgcggatctattctcgagtcagcggcaatcagatgccctcagtcatcgcggaggcacgtggactatggcagccagaaatcttctcttgaccttgtctctcccttcatggtcatggaaacgtgctatctagttgcgcactgtacttatccgatgactctattcagtttattggagacattcaccagcctcttcatgacgagggcctcgacttgggaggaaacgaaatcgatgtaacctttgacggggaatccaccaatctccaccacatcgttagtgccaccattctcacttacacccgtcatacatcaacacccgaacccacctccctatccccaaaaaccatcttctaaattaatagcagtgggacaccaacatccccgagaaatatgtcggagggtacgcgctctccgacgccaagtccttcgccgcgaccctcacgacctcgatcaaaaacggatcctataaatccctcaaagcgtcctggctcgacggcatcgacatatccgaccccgtcagctcggccacggattgggcttcggacgccaatgcttatgtctgcagcacggtcctgcccaatggtatttcagcggtcgaaaatacggatctgtcaggtgcgtactatacgaaggctattccggttgtggagttgcagatcgccaaagcggggtataggtatgtctgattagattttcttggtgttttccaaagggggtatacctgctaatgtctgcaggttggcggcttggttggatctcattgctactggtagtactagactttaa

SEQ ID NO: 62 - protein, 291 aa, Lachnellula sp.
MKTQAVASIFFLATSLPSALGWGTLGHQTVAYIATNFVSSATKTKFQSILDDTSTDYLASVATWADTYRYTDAGTWTAPFHFIDANDDPPSSCSVDYSRDCGTGGCVISAINNYTSRIQSTTLSDAEVKTAAKFIVHFIGDIHQPLHDEGLDLGGNEIDVTFDGESTNLHHIWDTNIPEKYVGGYALSDAKSFAATLTTSIKNGSYKSLKASWLDGIDISDPVSSATDWASDANAYVCSTVLPNGISAVENTDLSGAYYTKAIPVVELQIAKAGYRLAAWLDLIATGSTRL

SEQ ID NO: 63 - protein, 270 aa, Lachnellula sp.
WGTLGHQTVAYIATNFVSSATKTKFQSILDDTSTDYLASVATWADTYRYTDAGTWTAPFHFIDANDDPPSSCSVDYSRDCGTGGCVISAINNYTSRIQSTTLSDAEVKTAAKFIVHFIGDIHQPLHDEGLDLGGNEIDVTFDGESTNLHHIWDTNIPEKYVGGYALSDAKSFAATLTTSIKNGSYKSLKASWLDGIDISDPVSSATDWASDANAYVCSTVLPNGISAVENTDLSGAYYTKAIPVVELQIAKAGYRLAAWLDLIATGSTRL

SEQ ID NO: 64 - DNA, 1119 bp, Trichoderma reesei
  sig_peptide 1..57
  mat_peptide 58..1116
  source 1..1119: mol_type = genomic DNA
  CDS 1..64
  CDS 145..798
  CDS 854..1116
atgaagctctcaaatgccgtcgccttcagcctcgtctccctgccgggggctatggcctggggaggtgtgtatcgtctttcccccccgtgacacacattgtcaacaccatcaacgctagtctaacactcgtcttcctcgcatcaggtctcggccacatcacgaccgcctacattgccagcaacttcgtctccaactcgaccgaggcctacctgaagcagctcctgcgcagcaatgagtccgactacatggccaaggtagccagctgggccgattcgatccgatacacgaaatggggcaggttcacgagcacgttccacttcatcgacgcgcacgacaacccgcccgagtcgtgcaacgtcgactttgagcgcgactgcaaggagacgggctgcgtcatcaccgcgctggccaactacaccgagcagtcgctcgatcccgcgctgccggcgtggcggcgcgcccaggcggccaagttcgtcatccacttcgtcggcgacctgcaccagccgctgcacaacgaggacgtttcgaggggcggaaacggcatccacgtcaagtgggatggacgagactacaacctgcaccacgtctgggacagctccattgcggagaagctgattgggcgcgggaagccgtatctcgtggcgcagaagtggtctgctgccctcaccgagaaaattactggcggcgcgtatgccgacgagaaggagacttggctggcagacttggattttggcgactttgaggctacggcaatggcctggtcgcgggaatgcaacagcctcgtatgcgaatatggtaagctctaccttgagtatgcttcaaactgcaaagttatcttaccagcttttagtcttccccgagggccccaaagccatcgtcggccaggaactaagcggcgagtattatgaaaaggcggctcctatcctcgagaagcaggtcgcccgcgcaggttatcgaatggctgcatggctcgatcgcattgtggatgagtacaagaagagagaggccagctataccggcgaattgcccacggacgactatgccgaggagcctcttgatgagtttgacctcgagcttctagacgagacggagaagccctttgttggagagttgtaa

SEQ ID NO: 65 - protein, 327 aa, Trichoderma reesei
MKLSNAVAFSLVSLPGAMAWGGLGHITTAYIASNFVSNSTEAYLKQLLRSNESDYMAKVASWADSIRYTKWGRFTSTFHFIDAHDNPPESCNVDFERDCKETGCVITALANYTEQSLDPALPAWRRAQAAKFVIHFVGDLHQPLHNEDVSRGGNGIHVKWDGRDYNLHHVWDSSIAEKLIGRGKPYLVAQKWSAALTEKITGGAYADEKETWLADLDFGDFEATAMAWSRECNSLVCEYVFPEGPKAIVGQELSGEYYEKAAPILEKQVARAGYRMAAWLDRIVDEYKKREASYTGELPTDDYAEEPLDEFDLELLDETEKPFVGEL

SEQ ID NO: 66 - protein, 308 aa, Trichoderma reesei
WGGLGHITTAYIASNFVSNSTEAYLKQLLRSNESDYMAKVASWADSIRYTKWGRFTSTFHFIDAHDNPPESCNVDFERDCKETGCVITALANYTEQSLDPALPAWRRAQAAKFVIHFVGDLHQPLHNEDVSRGGNGIHVKWDGRDYNLHHVWDSSIAEKLIGRGKPYLVAQKWSAALTEKITGGAYADEKETWLADLDFGDFEATAMAWSRECNSLVCEYVFPEGPKAIVGQELSGEYYEKAAPILEKQVARAGYRMAAWLDRIVDEYKKREASYTGELPTDDYAEEPLDEFDLELLDETEKPFVGEL

SEQ ID NO: 67 - DNA, 1067 bp, Cordyceps cardinalis
  sig_peptide 1..57
  mat_peptide 58..1064
  source 1..1067: mol_type = genomic DNA
  CDS 1..64
  CDS 134..793
  CDS 868..1064
atgagattcaccgccgctgcctcatatggcctcctctcgctccctgcagcctctgcctggggaagtacgttgtcgaccccgcggaaatatgatggcaacacgtcatgccgaaaattgctaacatgctcattaggtcttgggcacattaccaccgcgtacatagccagcaactttgtctccaacaccactgaagcctatctcaaggacattctcatgcgcaatgacgaggactatctatcttctatagccagctgggctgattccatacggtacaccaaatggggccgcttcaccagcacctttcacttcatcgactcccacgacaacccccctcactcgtgcaccgtggaccttgagcgcgactgcaaggagacaggctgcgtcatctcggccctcgccaactataccgagcaggtctacgaccacgcgctgcccgcctggcgccgcgcgcaggccgccaagttcgtcgtgcactttgtcggcgacctgcaccagccgctgcacaacgaggacgtcgccaagggcggcaacggcatccacgtcctctgggacggccgcggctacaacctgcaccacgtctgggatagctccatcctggagaagtggctgggcggcctgcgcggcaagccctacgcgctggccaagcggtgggcggcgcagctcacggaagaaatcaccgacggcaagtgggcgcgagagaaggacggctggctgaaggacgtcaggttggataatgcaaacggtacggcgctggcgtggtcgcgcgagacgaatgccattgtctgttcacatggtgagttgctccttttgtcttgagtaccgcggggcttcgtttggggggcggacgatttcggctaacggaaccagtgtttccccagggtccagaggccataaggggccaagagctgggaggcaaatactatgaagaagcagcacctgttttggaaaggcaggtcgctcgggctggatatagaatggctgcttggcttgacgagattgccaatgagtatcacggccagaggagtatcgccaaggtcgacgcggagctggcccaactagaattgtga

SEQ ID NO: 68 - protein, 307 aa, Cordyceps cardinalis
MRFTAAASYGLLSLPAASAWGSLGHITTAYIASNFVSNTTEAYLKDILMRNDEDYLSSIASWADSIRYTKWGRFTSTFHFIDSHDNPPHSCTVDLERDCKETGCVISALANYTEQVYDHALPAWRRAQAAKFVVHFVGDLHQPLHNEDVAKGGNGIHVLWDGRGYNLHHVWDSSILEKWLGGLRGKPYALAKRWAAQLTEEITDGKWAREKDGWLKDVRLDNANGTALAWSRETNAIVCSHVFPQGPEAIRGQELGGKYYEEAAPVLERQVARAGYRMAAWLDEIANEYHGQRSIAKVDAELAQLEL

SEQ ID NO: 69 - protein, 288 aa, Cordyceps cardinalis
WGSLGHITTAYIASNFVSNTTEAYLKDILMRNDEDYLSSIASWADSIRYTKWGRFTSTFHFIDSHDNPPHSCTVDLERDCKETGCVISALANYTEQVYDHALPAWRRAQAAKFVVHFVGDLHQPLHNEDVAKGGNGIHVLWDGRGYNLHHVWDSSILEKWLGGLRGKPYALAKRWAAQLTEEITDGKWAREKDGWLKDVRLDNANGTALAWSRETNAIVCSHVFPQGPEAIRGQELGGKYYEEAAPVLERQVARAGYRMAAWLDEIANEYHGQRSIAKVDAELAQLEL

SEQ ID NO: 70 - DNA, 1103 bp, Acremonium alcalophilum
  sig_peptide 1..57
  mat_peptide 58..1100
  source 1..1103: mol_type = genomic DNA
  CDS 1..64
  CDS 129..788
  CDS 913..1100
atgaggttcctctcgatattaacggtactcggcgcatcggcctacggagctttagcatggggaagtgagtgatccgcagccgctacgccgatccgtcgaaaacactgctcacaaacgcgtaccatcaggtctcggtcacgtcacgacagcttacctagccagtcacctcgtctccaactcgaccgaagcgttctttcagaatctcctgcgcgacgaaacgagcgattacctcggcaaagtggccacgtgggccgacaccatccgctacacgcgctggggccggttcacgagcgtcttccacttcatcgacgccaaggacgagccccctacgtactgcggcgtcgaactcgcccgcgactgcaagcccgagggctgcgtcgtcacggccctagcgaactatacggcccggctcctcgacccggagctgccggcttgggagcgcaaccaggccgcccgcttcgttgtccactttgtcggcgacatccaccagccgttgcacgatgagaacgtggcccgtggcgggaacggcatctacgtgctgtggcagggccgccgattcaacctccattatgtctgggatagcgccattgccgagcggctgattgggcggaatcgccggaagccgtatgacaatgcgaagtggtgggcggaagagcttgccgtccagatccgggagggcaagtttgtcgaggagaaggagggttggttgaaggatgttgacattgatgatgcgactgcgacggcgctggcatgggctcgggaaggcaacgcctatgtctgttcgcacggtgagtattttttctttcttcgttctttctgtcttttctgatgctgctgagcttgatggaatacttatcctccccccttctgccgtgtgacgttgacaacagctgatcgtcctgcgtttcgtagtgttgccagaaggccctcttgccatcgagggacaggaactcagcggagagtactatgataaggcggcccccgttgttgagttgcagattgcccgtgccgggtaccggatggctgcctggctagatctcattgcgaggcggttccaggagcaaggagccgagagggaggaagatctttgggaggagctataa

SEQ ID NO: 71 - protein, 304 aa, Acremonium alcalophilum
MRFLSILTVLGASAYGALAWGSLGHVTTAYLASHLVSNSTEAFFQNLLRDETSDYLGKVATWADTIRYTRWGRFTSVFHFIDAKDEPPTYCGVELARDCKPEGCVVTALANYTARLLDPELPAWERNQAARFVVHFVGDIHQPLHDENVARGGNGIYVLWQGRRFNLHYVWDSAIAERLIGRNRRKPYDNAKWWAEELAVQIREGKFVEEKEGWLKDVDIDDATATALAWAREGNAYVCSHVLPEGPLAIEGQELSGEYYDKAAPVVELQIARAGYRMAAWLDLIARRFQEQGAEREEDLWEEL

SEQ ID NO: 72 - protein, 285 aa, Acremonium alcalophilum
WGSLGHVTTAYLASHLVSNSTEAFFQNLLRDETSDYLGKVATWADTIRYTRWGRFTSVFHFIDAKDEPPTYCGVELARDCKPEGCVVTALANYTARLLDPELPAWERNQAARFVVHFVGDIHQPLHDENVARGGNGIYVLWQGRRFNLHYVWDSAIAERLIGRNRRKPYDNAKWWAEELAVQIREGKFVEEKEGWLKDVDIDDATATALAWAREGNAYVCSHVLPEGPLAIEGQELSGEYYDKAAPVVELQIARAGYRMAAWLDLIARRFQEQGAEREEDLWEEL

SEQ ID NO: 73 - DNA, 1057 bp, Microdochium phragmitis
  sig_peptide 1..60
  mat_peptide 61..1054
  source 1..1057: mol_type = genomic DNA
  CDS 1..73
  CDS 144..1054
atgcgtactgcccacgtcgctgccgtcgcggccggccttctggccagcccagccatgtcctggaatacggatggtcagcatactctctcctatcccacaaaaccctaatcataaagtacctttgctaacatatcaactcatagtgcaccaacaaatcgcctacgctgccgacgagctcctaaccccctacaccaaacaaatcatccgccagctcctcgagccctccgctcagggctccctggggcgcgtcggcgcctgggccgacggctaccgcaagacccccgagggcgcctacacggatacatggcactacatcgacccggctgacaacccgccctccttctgcaatgtgcacttcaaccgcgactgctccaagaagggctgcatcgtctccgccctgacgaaccagaccgagatcgccaagggctgcatcacccgcgcgaaacacggccagatccgcaacggcgaggacgccacctgcgccaacgcgatcaaattcattgcgcatttcacgggtgatgccacgcagcctttacacgtctccggcatcgcggccggtggaaacgggttcaacgttactttcgcgggcaaagccaccaatctccactccgtctgggacggcgccatcatctacaaattcgccaacgtctccggtgacccgacccaaaacatccccgccggattcaaaaacgatacactccagccatttttcaaaaacaccatcaagcgtctccggagcgatagtttcatcacccccgttgcggatatgatttcctgcacggacccggggacaccgcagaagtgtgcgatggagtgggccaaggatacgaacgagtggacgtgtgattatgtgtatagtcagcttttcaatgggacggatttggcggatagtgggtatgctaaaggagctgggtatattgttgatgtgcagactgcgaaagcggcggtgaggatggcgacgtggttcaacaggcttgtggagggacggtatgggcagagggaggtgcatttggatttggtgccgagctgggtttttgggcctgttaagggtgcttaa

SEQ ID NO: 74 - protein, 328 aa, Microdochium phragmitis
MRTAHVAAVAAGLLASPAMSWNTDVHQQIAYAADELLTPYTKQIIRQLLEPSAQGSLGRVGAWADGYRKTPEGAYTDTWHYIDPADNPPSFCNVHFNRDCSKKGCIVSALTNQTEIAKGCITRAKHGQIRNGEDATCANAIKFIAHFTGDATQPLHVSGIAAGGNGFNVTFAGKATNLHSVWDGAIIYKFANVSGDPTQNIPAGFKNDTLQPFFKNTIKRLRSDSFITPVADMISCTDPGTPQKCAMEWAKDTNEWTCDYVYSQLFNGTDLADSGYAKGAGYIVDVQTAKAAVRMATWFNRLVEGRYGQREVHLDLVPSWVFGPVKGA

SEQ ID NO: 75 - protein, 308 aa, Microdochium phragmitis
WNTDVHQQIAYAADELLTPYTKQIIRQLLEPSAQGSLGRVGAWADGYRKTPEGAYTDTWHYIDPADNPPSFCNVHFNRDCSKKGCIVSALTNQTEIAKGCITRAKHGQIRNGEDATCANAIKFIAHFTGDATQPLHVSGIAAGGNGFNVTFAGKATNLHSVWDGAIIYKFANVSGDPTQNIPAGFKNDTLQPFFKNTIKRLRSDSFITPVADMISCTDPGTPQKCAMEWAKDTNEWTCDYVYSQLFNGTDLADSGYAKGAGYIVDVQTAKAAVRMATWFNRLVEGRYGQREVHLDLVPSWVFGPVKGA

SEQ ID NO: 76 - DNA, 1200 bp, Bacillus deramificans
  sig_peptide 1..84
  mat_peptide 85..1197
  source 1..1200: mol_type = genomic DNA
  CDS 1..1197
atgaaactgacaaaaaaaatatcttttgctttattagttacttttttgttttcaatggttagtgcttgcagtgatgtgagtagccagtcacaaccaactaaaaataaccaagtagccattacaaaaacaaatgttaaatcagacattacgcaaagcaaaaaagcgacagataaaagttcgacaactagtagttctactcaaaagtctactactcattcaacaaatgtaaaaaaggattcagctacaaccaataattctaattcagaagatcaatataaaacacttgcggacttagattttaaggggaaacaagttattatattgaataataatcatgctgttttttctaaaaatgacttaagtttagctaaaggaagctggcaatctttttctaatttagattctttaaatcgtgtcggtgcagcaagtgccatgttatcgaagtctctaatgcctactgcagaacgtgagccactttatgtcgatcctacaggttggaagaataaaaaaatcattgttaatggtaaatcggaatggctttataatcgttctcaccttattggttatcaatttacaggcgaaaataacaatcctaaaaatttaatgacaggaacaagaagtcttaatgaccccgatatgcttaaatatgaagacgaagtggcctcttatttaagaacaaccaatcaccatgtccgttaccaagtagaacctatatttagaggggatgaattggtagctagaggcgttcatatgcaagccaaaagtatagaagataataaaattgattttaatgtttatatttttaatgtcgaacaaggtgtaaccatcaattacgctgatggttcttctagagtgcaatctggtgcaacaataaataataatacatcttctactacaacgaagaaaagcactagcaactccaatgcgagctctacaagtgtttctacgagtgaattaacggtaaatgatggggatcaagcaaccgtgtcagttaaaacaaaaccaaatgtgcaaggaactatagaagtagattattcttctggtcctagccatgcatccggcttaggacctaagacatctgatcgtagcggtaatataagttggacttgggcagttggtacaaggacaaaaccaggaacttataatgtaatcattacagtcaatggacaaaccatcacaaaacatttagttgtaaaatga

SEQ ID NO: 77 - protein, 399 aa, Bacillus deramificans
MKLTKKISFALLVTFLFSMVSACSDVSSQSQPTKNNQVAITKTNVKSDITQSKKATDKSSTTSSSTQKSTTHSTNVKKDSATTNNSNSEDQYKTLADLDFKGKQVIILNNNHAVFSKNDLSLAKGSWQSFSNLDSLNRVGAASAMLSKSLMPTAEREPLYVDPTGWKNKKIIVNGKSEWLYNRSHLIGYQFTGENNNPKNLMTGTRSLNDPDMLKYEDEVASYLRTTNHHVRYQVEPIFRGDELVARGVHMQAKSIEDNKIDFNVYIFNVEQGVTINYADGSSRVQSGATINNNTSSTTTKKSTSNSNASSTSVSTSELTVNDGDQATVSVKTKPNVQGTIEVDYSSGPSHASGLGPKTSDRSGNISWTWAVGTRTKPGTYNVIITVNGQTITKHLVVK

SEQ ID NO: 78 - protein, 371 aa, Bacillus deramificans
QSQPTKNNQVAITKTNVKSDITQSKKATDKSSTTSSSTQKSTTHSTNVKKDSATTNNSNSEDQYKTLADLDFKGKQVIILNNNHAVFSKNDLSLAKGSWQSFSNLDSLNRVGAASAMLSKSLMPTAEREPLYVDPTGWKNKKIIVNGKSEWLYNRSHLIGYQFTGENNNPKNLMTGTRSLNDPDMLKYEDEVASYLRTTNHHVRYQVEPIFRGDELVARGVHMQAKSIEDNKIDFNVYIFNVEQGVTINYADGSSRVQSGATINNNTSSTTTKKSTSNSNASSTSVSTSELTVNDGDQATVSVKTKPNVQGTIEVDYSSGPSHASGLGPKTSDRSGNISWTWAVGTRTKPGTYNVIITVNGQTITKHLVVK

SEQ ID NO: 79 - DNA, 783 bp, Bacillus thuringiensis
  sig_peptide 1..75
  mat_peptide 76..780
  source 1..783: mol_type = genomic DNA
  CDS 1..780
atgtttaaaagtaaagtattaaacagtaaactagtaaaaacattagccttaactacagggattttaggatttgcaactggttgtgataacttagtagaccaagctactcaaacatttaaagccgaccacggagttaaaacagaagaggtagcacaagcgaagaaaccaaaagttacagatttaaagaaaatcgcaacgagttacaacggtgaaaaggtagtaacagttaacaacaataaagcagactttacacaagaccaattagataaggtaagattaactcaaactgaccctacatggcaagaatactcgaatttagataatatgaatagagtaggggtagcaacggcattacttggaatgcagaatcaaccgaaggaaaagcgtgacgatagattaaatacaaaacctacaggttggcatcaaaagaaactaagtgatggtagctacttgtatgatagaagtcatctaattggattccagttatcaggacaaaacgataacccgaaaaaccttatgacaggtacaaaagactttaatcgtaagagcatgttaaaatatgagaacatggtagacaaagaagtagaaaaaggaagctacgtcttatatgaagtaaaacccgtattcattggaagtgaaaaggttgctcgtggtgtaaacatgaaagcaaaatcaatgaacaatcaaaatttagaattcaacgtattttgtcataatgtacaagatggggtagaaataaactacgaagacggaacttctaaactaacaggtaagttgaaataa

SEQ ID NO: 80 - protein, 260 aa, Bacillus thuringiensis
MFKSKVLNSKLVKTLALTTGILGFATGCDNLVDQATQTFKADHGVKTEEVAQAKKPKVTDLKKIATSYNGEKVVTVNNNKADFTQDQLDKVRLTQTDPTWQEYSNLDNMNRVGVATALLGMQNQPKEKRDDRLNTKPTGWHQKKLSDGSYLYDRSHLIGFQLSGQNDNPKNLMTGTKDFNRKSMLKYENMVDKEVEKGSYVLYEVKPVFIGSEKVARGVNMKAKSMNNQNLEFNVFCHNVQDGVEINYEDGTSKLTGKLK

SEQ ID NO: 81 - protein, 235 aa, Bacillus thuringiensis
TGCDNLVDQATQTFKADHGVKTEEVAQAKKPKVTDLKKIATSYNGEKVVTVNNNKADFTQDQLDKVRLTQTDPTWQEYSNLDNMNRVGVATALLGMQNQPKEKRDDRLNTKPTGWHQKKLSDGSYLYDRSHLIGFQLSGQNDNPKNLMTGTKDFNRKSMLKYENMVDKEVEKGSYVLYEVKPVFIGSEKVARGVNMKAKSMNNQNLEFNVFCHNVQDGVEINYEDGTSKLTGKLK

SEQ ID NO: 82 - DNA, 975 bp, Penicillium virgatum
  sig_peptide 1..66
  mat_peptide 67..972
  source 1..975: mol_type = genomic DNA
  CDS 1..972
atgtccaaagcaaccttggccgtcatcgccgccgccagtgcagccactggtgctggtgtcactgcactcctttactccggcaaaccctccaagccgcatcaacagcaacaacttccaacacccactaccccaggtgtcaaagctccctcaaccatcgcacccccaaccctagcagccaaagtcaccggccccgtcgatccagccggagtcctccaatatggcttcccaggcccaatcgccgacgagctctcctccctccccctacacggtgcttatgaccgtcgcactcgcaatccctcctgggtagccgagcacatcacaccagcatccctggcaatcaacaacgcagaccgaaagaagagcaccttcttcgaagacaccaccgtcccagccatgttccgcgccaaactgtccgattacttccgctctggctacgaccgcggccaccaggttcccgcggcagacgcaaaatggtcccaggatgccatggacggtactttcgcgctctcaaacatgtgtccccaggtcggagagggcttcaaccgcgactactgggctcactttgagactttctgccgggatctcaccaagacctacccatccgttcgcatcgttacgggcccgctgtacctgcctcaccgtgaccctgatggaaagtggcgtgtcaactatgaggttattggcactccgcccaatgtcgctgttcccacgcatttctataaggtcatctatgccgaggacggaactaactcgcctactgccaaggttgcgctcggtgcttttgttctgcccaatgctcgcattgcgaatgataagcgtctgacggactttgaggttcctcttgaggctgttgagcgtgcttctggtttgcagtttggttcgcagttggacctcagccgtcgtcgtcgcctgtgccaggaggttgcttgcgaggttactgtccgcgagttcaacaatgctaagaagcgttcttga

SEQ ID NO: 83 - protein, 324 aa, Penicillium virgatum
MSKATLAVIAAASAATGAGVTALLYSGKPSKPHQQQQLPTPTTPGVKAPSTIAPPTLAAKVTGPVDPAGVLQYGFPGPIADELSSLPLHGAYDRRTRNPSWVAEHITPASLAINNADRKKSTFFEDTTVPAMFRAKLSDYFRSGYDRGHQVPAADAKWSQDAMDGTFALSNMCPQVGEGFNRDYWAHFETFCRDLTKTYPSVRIVTGPLYLPHRDPDGKWRVNYEVIGTPPNVAVPTHFYKVIYAEDGTNSPTAKVALGAFVLPNARIANDKRLTDFEVPLEAVERASGLQFGSQLDLSRRRRLCQEVACEVTVREFNNAKKRS

SEQ ID NO: 84 - protein, 302 aa, Penicillium virgatum
LLYSGKPSKPHQQQQLPTPTTPGVKAPSTIAPPTLAAKVTGPVDPAGVLQYGFPGPIADELSSLPLHGAYDRRTRNPSWVAEHITPASLAINNADRKKSTFFEDTTVPAMFRAKLSDYFRSGYDRGHQVPAADAKWSQDAMDGTFALSNMCPQVGEGFNRDYWAHFETFCRDLTKTYPSVRIVTGPLYLPHRDPDGKWRVNYEVIGTPPNVAVPTHFYKVIYAEDGTNSPTAKVALGAFVLPNARIANDKRLTDFEVPLEAVERASGLQFGSQLDLSRRRRLCQEVACEVTVREFNNAKKRS

SEQ ID NO: 85 - DNA, 825 bp, Streptomyces cirratus
  sig_peptide 1..84
  mat_peptide 85..822
  source 1..825: mol_type = genomic DNA
  CDS 1..822
atggataaaaaaacaaaacagacactgatcggactgctagtgttattgcttttatctgcgggaagctattatatcaagcagatgcagtcgacagctaatagtcccaacaccaagcttagtcagaaaaaacaagcgtctgaagctcctagtcaagcattggcagagagtgtcttaacagacgcagtcaagagtcaagtaaaagggagtctggagtggaatggctcaggtgcttttatcattaatggtaataaaacaaatctagatgccaaggtttcaagtaagccctacgctgataataaaacaaagacagtgggcaaggaaacagtgccaaccgtagcaaatgccctcttgtctaaggctactcgccagtacaagaatcgtgaagaaactgggaatggttcgacttcatggactcctccaggctggcatcaggttaagaatctaaagggtacttatacccatgcggtcgatagaggtcacttgttaggctatgccttaatcggtggcttggatggttttgatgcctcgacaagcaatcctaaaaacattgctgttcaaacagcctgggcaaatcaggcgcaagccgaggattcgactggtcaaaactactatgaaagcatggtacgtaaggccttggaccaaaacaagcgtgtccgttatcgtgtaaccctttattacgcttcaaatgaggatttagttccttcagcttcacagattgaagccaagtcttcagacggagaattggaattcaatgttctagttcccaatgttcaaaagggacttcaactggattaccgaactggagaagtaacggtaactcagtaa

SEQ ID NO: 86 - protein, 274 aa, Streptomyces cirratus
MDKKTKQTLIGLLVLLLLSAGSYYIKQMQSTANSPNTKLSQKKQASEAPSQALAESVLTDAVKSQVKGSLEWNGSGAFIINGNKTNLDAKVSSKPYADNKTKTVGKETVPTVANALLSKATRQYKNREETGNGSTSWTPPGWHQVKNLKGTYTHAVDRGHLLGYALIGGLDGFDASTSNPKNIAVQTAWANQAQAEDSTGQNYYESMVRKALDQNKRVRYRVTLYYASNEDLVPSASQIEAKSSDGELEFNVLVPNVQKGLQLDYRTGEVTVTQ

SEQ ID NO: 87 - protein, 246 aa, Streptomyces cirratus
QSTANSPNTKLSQKKQASEAPSQALAESVLTDAVKSQVKGSLEWNGSGAFIINGNKTNLDAKVSSKPYADNKTKTVGKETVPTVANALLSKATRQYKNREETGNGSTSWTPPGWHQVKNLKGTYTHAVDRGHLLGYALIGGLDGFDASTSNPKNIAVQTAWANQAQAEDSTGQNYYESMVRKALDQNKRVRYRVTLYYASNEDLVPSASQIEAKSSDGELEFNVLVPNVQKGLQLDYRTGEVTVTQ

SEQ ID NO: 88 - DNA, 1032 bp, Acremonium sp.
  sig_peptide 1..63
  mat_peptide 64..1029
  source 1..1032: mol_type = genomic DNA
  CDS 1..1029
atgtcgaaaacctccgccgccatcgccctcctcggtgcgggagcaggcgcctccatcaccgcagccgtcctctcgctccgctcctcaaacaacgctgccaagcccgagatccccatcgcctcgcaaaacaccacagccatgcccccgaccccaacattaccaccgacccgcacgacagtgccgccgcccgcccccctcccaggcagcgcacccttccctcccacctcctcgaccgcccccgtcgatcccgccggcctcttccagtacggcttccccggcccggtcgccgacctcgccacccgcgactccctcgtctcctcctacgaccgccgcctccgcaacccccactgggtcgtcgagcacatcaccccggcctccctcgccgcccgcaacgccgaccgcaaaaactccgtcttcgccgaggacccctccgtcccgcccaagttccgcgccctcctcaaggactacttccgctccggctacgaccgcggccaccaggtccccgccgccgacgcgaaatggtcccagcgcgccatggacgacaccttctacctctccaacatgtgcccccaggtcggcgagggcttcaaccgcgactactgggcgcactttgaggacttttgccgccgcctcaccgtccgctacccctccgtccgcatcgtcacgggacccctctacctccccaagaaggacccaacagacaacaagtggtacgtcaagtacgagatgatcgggaacccgccctccgtcgccgtgccgacgcacttttacaaggtcatcctcgccgaggacggccgcccggggggcagcgtggccgtcggcgccttcgtcctgcccaacgccgtcatcgacaacagcaagcccatcaccgactttgaggtcccgctcgaggccgtcgagagggcgagcgggctcgagtttgcgagcaagctcgacgtgagcaggcgcaagaggctgtgtacggacacgacgtgtgcgctggtgatcaaagactatgctaagaggcaggaggctttcgggaagaagaactga

SEQ ID NO: 89 - protein, 343 aa, Acremonium sp.
MSKTSAAIALLGAGAGASITAAVLSLRSSNNAAKPEIPIASQNTTAMPPTPTLPPTRTTVPPPAPLPGSAPFPPTSSTAPVDPAGLFQYGFPGPVADLATRDSLVSSYDRRLRNPHWVVEHITPASLAARNADRKNSVFAEDPSVPPKFRALLKDYFRSGYDRGHQVPAADAKWSQRAMDDTFYLSNMCPQVGEGFNRDYWAHFEDFCRRLTVRYPSVRIVTGPLYLPKKDPTDNKWYVKYEMIGNPPSVAVPTHFYKVILAEDGRPGGSVAVGAFVLPNAVIDNSKPITDFEVPLEAVERASGLEFASKLDVSRRKRLCTDTTCALVIKDYAKRQEAFGKKN

SEQ ID NO: 90 - protein, 322 aa, Acremonium sp.
AVLSLRSSNNAAKPEIPIASQNTTAMPPTPTLPPTRTTVPPPAPLPGSAPFPPTSSTAPVDPAGLFQYGFPGPVADLATRDSLVSSYDRRLRNPHWVVEHITPASLAARNADRKNSVFAEDPSVPPKFRALLKDYFRSGYDRGHQVPAADAKWSQRAMDDTFYLSNMCPQVGEGFNRDYWAHFEDFCRRLTVRYPSVRIVTGPLYLPKKDPTDNKWYVKYEMIGNPPSVAVPTHFYKVILAEDGRPGGSVAVGAFVLPNAVIDNSKPITDFEVPLEAVERASGLEFASKLDVSRRKRLCTDTTCALVIKDYAKRQEAFGKKN

SEQ ID NO: 91 - DNA, 1092 bp, Daldinia fissa
  sig_peptide 1..54
  mat_peptide 55..1089
  source 1..1092: mol_type = genomic DNA
  CDS 1..1089
atgtcaaaaactacgttagcagttatcgcagcggcaagcgcaggagccggcgccgccatcacagctactatgtacgggttacgatcagaccgtaatctcgaaagcaagactatatccttaacgacgactacctcctcggcctcacctacggcgggaggctccgcgccgatccccatcccagctaaacagatatttactcccccgaaccaacaaaccgcgcagacgggcctgtcgacctcggcccccgccgccgtgaaccttggacccgtcgacccggccggcctcttcgaatatggcttcccgggcccggtagccgacatcgcgacgcggcaagcgctgatctcttcgttcgaccgccgactgcgcaacccgcactgggtagcagagcacatcacgccagcgtcgctagcaattcgcgacggcgaccgcaagcacagcacattcctagaagacgaggccgtgcccgagaagttccgcgcgcggctaaaggactacttccgctcgggctacgaccggggccaccaagtccccgcggccgacgcgcgctggggccaagccgccatggacgagaccttcttcctgaccaacatgtgcccgcaggtcggcgagggcttcaaccgggactactgggcgcacttcgaggacttctgccggggcctgacgtcgcggtatcccagcgtgcgcgtcgtcacgggcccgctatacctgcccaagcgcgaccccacagacaacaagtggtacgtgcggtacgaagtcatcggcaacccgcccaacgtcgccgtccccacgcacttctacaaggtcatattcgccgaagacggagccgtcggcggcaacgtagccctcggcgccttcgtactgcccaacgcccccatccccaacgacaagcccctgacagacttcgaagtccccgtcgaggccgtcgagcgcgccagcggcctcgaattcgccaccaagctccccgcccagcgccgcaggcgcctctgcaccgaagccccctgcgccctcgtcatcaaagactacgccaaccggcaaaaggcgtttgcgaagccccagcagggcgcttcttcttccgccgtcgcgaagcgctag

SEQ ID NO: 92 - protein, 363 aa, Daldinia fissa
MSKTTLAVIAAASAGAGAAITATMYGLRSDRNLESKTISLTTTTSSASPTAGGSAPIPIPAKQIFTPPNQQTAQTGLSTSAPAAVNLGPVDPAGLFEYGFPGPVADIATRQALISSFDRRLRNPHWVAEHITPASLAIRDGDRKHSTFLEDEAVPEKFRARLKDYFRSGYDRGHQVPAADARWGQAAMDETFFLTNMCPQVGEGFNRDYWAHFEDFCRGLTSRYPSVRVVTGPLYLPKRDPTDNKWYVRYEVIGNPPNVAVPTHFYKVIFAEDGAVGGNVALGAFVLPNAPIPNDKPLTDFEVPVEAVERASGLEFATKLPAQRRRRLCTEAPCALVIKDYANRQKAFAKPQQGASSSAVAKR

SEQ ID NO: 93 - protein, 345 aa, Daldinia fissa
AITATMYGLRSDRNLESKTISLTTTTSSASPTAGGSAPIPIPAKQIFTPPNQQTAQTGLSTSAPAAVNLGPVDPAGLFEYGFPGPVADIATRQALISSFDRRLRNPHWVAEHITPASLAIRDGDRKHSTFLEDEAVPEKFRARLKDYFRSGYDRGHQVPAADARWGQAAMDETFFLTNMCPQVGEGFNRDYWAHFEDFCRGLTSRYPSVRVVTGPLYLPKRDPTDNKWYVRYEVIGNPPNVAVPTHFYKVIFAEDGAVGGNVALGAFVLPNAPIPNDKPLTDFEVPVEAVERASGLEFATKLPAQRRRRLCTEAPCALVIKDYANRQKAFAKPQQGASSSAVAKR

SEQ ID NO: 94 - DNA, 976 bp, Actinomucor elegans
  sig_peptide 1..75
  mat_peptide 76..973
  source 1..976: mol_type = genomic DNA
  CDS 1..160
  CDS 222..973
atggcgatacccttggcacattacgtttggttaactagcggcttctttttgggcatgttcttcatgtacaatctcgtgccatttcgagtgaatgagcaccatgcccacttgcaacccagacagaaactagccaacagcgtactcaaattcggaaacccaggtgagctgcatataggatacagcaaagacgcattaaaaataatagtctaacttgatgttagggcctatcaatgacttgctcgaaagaacggcttacaccgcttcctataaccgcaaagaccgtatacctcattgggtcggagaacatcttactgctgacagtctagttgctggtgagggtgtgacacgcgataaatctcgttttaaagatgacggcgctgtccctgatcttttcaaggtatttacaaaagactataccaacagtggctatgaccgcggtcatatggctcctgctggcgacgctgttgctacccagcaagccatggatgaaacatttttgttgaccaacattgcaccacaaattggccccggattcaatcgacaatactgggcttatctggaaaaattctgtcgtgagttgaccaagaactatacggacgtgtatgtctatacagggcctttgtttctgcctcaactgcaaggacaaagtgtcaaggtggatcttgaagatgacaaggtggccgtgtcacaggcagctggtaaaaaatacaagatgcagtatgatatgattggtagcgcaggccctacgatcgccgtaccaacgcattatttcaagatcctgcttgtaaatcacgcagacgagtttatattggcggcttttgtgttaccgaatcaggctatcgatcataagatcccattgtctcagtttcaggtgggtttagaggctattgaaaaggcttctggtctagtgttctttgatcaattggatcgaaccacattcaaaaatttgtgtgatcaagtacaatgcatcgtttaa

SEQ ID NO: 95 - protein, 304 aa, Actinomucor elegans
MAIPLAHYVWLTSGFFLGMFFMYNLVPFRVNEHHAHLQPRQKLANSVLKFGNPGPINDLLERTAYTASYNRKDRIPHWVGEHLTADSLVAGEGVTRDKSRFKDDGAVPDLFKVFTKDYTNSGYDRGHMAPAGDAVATQQAMDETFLLTNIAPQIGPGFNRQYWAYLEKFCRELTKNYTDVYVYTGPLFLPQLQGQSVKVDLEDDKVAVSQAAGKKYKMQYDMIGSAGPTIAVPTHYFKILLVNHADEFILAAFVLPNQAIDHKIPLSQFQVGLEAIEKASGLVFFDQLDRTTFKNLCDQVQCIV

SEQ ID NO: 96 - protein, 279 aa, Actinomucor elegans
VPFRVNEHHAHLQPRQKLANSVLKFGNPGPINDLLERTAYTASYNRKDRIPHWVGEHLTADSLVAGEGVTRDKSRFKDDGAVPDLFKVFTKDYTNSGYDRGHMAPAGDAVATQQAMDETFLLTNIAPQIGPGFNRQYWAYLEKFCRELTKNYTDVYVYTGPLFLPQLQGQSVKVDLEDDKVAVSQAAGKKYKMQYDMIGSAGPTIAVPTHYFKILLVNHADEFILAAFVLPNQAIDHKIPLSQFQVGLEAIEKASGLVFFDQLDRTTFKNLCDQVQCIV

SEQ ID NO: 97 - DNA, 1011 bp, Talaromyces leycettanus
  sig_peptide 1..51
  mat_peptide 52..1008
  source 1..1011: mol_type = genomic DNA
  CDS 1..1008
atgtcaaaggccacgatagcgatcatcgctgcagccagtgctgccactggcgctggtgtcaccgctcttctctactcctccaaatcctctcgccagcagtcagctcctgcagctcttcctccccccgctgcgcccgctcctctgaccaagactgctgttcctgttccctccgcccatgtcccagagccgactcttgctcccaaaccctccaccgctgctccggtcaatccctcgggcatcttccaatacggctttcccggccctgtagctgaccctctcaactccctgcctttgaccggagcatacgatcgtcgcactcgcaatccggcctgggttgccgaacacatcactccccaatcgctcgctctcggcaacgcggaccgcaagaacagtgtgttctttgaggatgtcactatcccggtcgccttccgtgccaagttggccgactacttccgctccggttatgatcgcggccatcaggtgcccgccgcggacgccaaatggtcgcaggatgcgatggacgcgaccttcgctttgaccaacatgtgtccgcaggtcggagagggattcaacagggactactgggcccattttgaagatttctgccgcaacctggccaagaagtatccttcggttcgcattgtcaccggtcctctgtacctgcctcaccgtgaccctgacggaaaatggcgcgtgagctacgaagtgattggcaacccacctaacgtcgcagtcccgacgcatttctacaaggtcatctacggtgaagacggtactggcagccccaacagcagcgtcgcattgggtgcgtttgtcctgcccaatgccgtcattccgaataccaagagcttggccgatttcgaagtgcctctggaagctgtggagcgtgccagcggtctggaatttgcgtccaagttggatgtcagccgccgtaagcgcctttgccaggaggtgaagtgcgagattacggtgcgcgagttcaacaataccaacaagaagctcctgaagtga

SEQ ID NO: 98 - protein, 336 aa, Talaromyces leycettanus
MSKATIAIIAAASAATGAGVTALLYSSKSSRQQSAPAALPPPAAPAPLTKTAVPVPSAHVPEPTLAPKPSTAAPVNPSGIFQYGFPGPVADPLNSLPLTGAYDRRTRNPAWVAEHITPQSLALGNADRKNSVFFEDVTIPVAFRAKLADYFRSGYDRGHQVPAADAKWSQDAMDATFALTNMCPQVGEGFNRDYWAHFEDFCRNLAKKYPSVRIVTGPLYLPHRDPDGKWRVSYEVIGNPPNVAVPTHFYKVIYGEDGTGSPNSSVALGAFVLPNAVIPNTKSLADFEVPLEAVERASGLEFASKLDVSRRKRLCQEVKCEITVREFNNTNKKLLK

SEQ ID NO: 99 - protein, 319 aa, Talaromyces leycettanus
AGVTALLYSSKSSRQQSAPAALPPPAAPAPLTKTAVPVPSAHVPEPTLAPKPSTAAPVNPSGIFQYGFPGPVADPLNSLPLTGAYDRRTRNPAWVAEHITPQSLALGNADRKNSVFFEDVTIPVAFRAKLADYFRSGYDRGHQVPAADAKWSQDAMDATFALTNMCPQVGEGFNRDYWAHFEDFCRNLAKKYPSVRIVTGPLYLPHRDPDGKWRVSYEVIGNPPNVAVPTHFYKVIYGEDGTGSPNSSVALGAFVLPNAVIPNTKSLADFEVPLEAVERASGLEFASKLDVSRRKRLCQEVKCEITVREFNNTNKKLLK

SEQ ID NO: 100 - DNA, 31 bp, Synthetic construct
  source 1..31: mol_type = other DNA
gacgcggccgcaccatgccgcgcttactccc

SEQ ID NO: 101 - DNA, 28 bp, Synthetic construct
  source 1..28: mol_type = other DNA
gacgcgatcgctcaagagggctgactcg

SEQ ID NO: 102-104 - skipped (3 entries)

SEQ ID NO: 105 - protein, 27 aa, Synthetic construct
  REGION 1..27: note = Signal peptide
MKKPLGKIVASTALLISVAFSSSIASA

SEQ ID NO: 106 - DNA, 789 bp, Bacillus sp.
  sig_peptide 1..93
  mat_peptide 94..786
  source 1..789: mol_type = genomic DNA
  CDS 1..786
atgaaacttaaaacaacacttataaaatccatttcaattatcgcagcaagtatgatgctgggagcttgtagtcctccttctaatgctagttccagtacgaataaacaagaaccatctcaagctaccacgaaacaagaatctaatcagactcaaaataagacttctaacggccaacaacagtcttataacatagaggacattgcaaagaactacaaaggtcaaaaagtagtagaaataaacggaaataaagctgattttacacaagatcaattagataaagtacagttgaagaatacaaatcctacatggcaagagttctctaacttagatagtaagaacagagttggagtagcaacagcattaattggtaaagaaattcaacctaaagaaaaacgagatgagagattgaatacaaaacctactggttggcatcaaaagaaattaagtgatggtagtacattgtttgatagaagtcatttaattggatatcaactaactggtcaaaacgacaatcccaagaatttaatgactggtacaaaagattttaaccgacatagtatgttaaagtatgaaaacatggtagataaagaggttgaaaaaggaagttatgtactttacgaagtaaaaccagtatttatcggtgacgagttagtcgcaagaggtgtacaaatgaaagcgaaaacggttaataataatcacttagatttcaacgtattctgttttaatgtgcaagatggtgtagagattgactataaagatggtacttctaaactagttaataaacaataa

SEQ ID NO: 107 - protein, 262 aa, Bacillus sp.
MKLKTTLIKSISIIAASMMLGACSPPSNASSSTNKQEPSQATTKQESNQTQNKTSNGQQQSYNIEDIAKNYKGQKVVEINGNKADFTQDQLDKVQLKNTNPTWQEFSNLDSKNRVGVATALIGKEIQPKEKRDERLNTKPTGWHQKKLSDGSTLFDRSHLIGYQLTGQNDNPKNLMTGTKDFNRHSMLKYENMVDKEVEKGSYVLYEVKPVFIGDELVARGVQMKAKTVNNNHLDFNVFCFNVQDGVEIDYKDGTSKLVNKQ

SEQ ID NO: 108 - protein, 231 aa, Bacillus sp.
STNKQEPSQATTKQESNQTQNKTSNGQQQSYNIEDIAKNYKGQKVVEINGNKADFTQDQLDKVQLKNTNPTWQEFSNLDSKNRVGVATALIGKEIQPKEKRDERLNTKPTGWHQKKLSDGSTLFDRSHLIGYQLTGQNDNPKNLMTGTKDFNRHSMLKYENMVDKEVEKGSYVLYEVKPVFIGDELVARGVQMKAKTVNNNHLDFNVFCFNVQDGVEIDYKDGTSKLVNKQ

SEQ ID NO: 109 - DNA, 828 bp, Streptococcus infantis
  sig_peptide 1..90
  mat_peptide 91..825
  source 1..828: mol_type = genomic DNA
  CDS 1..825
atgaataaaaaaacaaggcaggcactcataggtcttctgcttttcctacttttgtcggcaggaagttactacataaaagaaatgcaggcttccaatgctacaccgcaaacacaggttaaccagaaatcccagtcgcttgatactccaagtcaaaaattggccgagagtgtactgactgattcggtaaaaaagcaaataaagggtactcttgagtggaatggatcaggtgcctttatcgtaaatgggaacaaaacgaacttagatgcaaaggtttcaagtaaaccctatgctgataataagacaaaaactgtaggtggggagacggttccaactgtggctaacgccctcatgtcaaaagcaacaagacagtataaagaccgtgaagaaaccgggaatggttcaacttcttggaccccagcaggatggcatcaagtcaaaaacctaaaagggacttataatcacgctgtagatagaggacatttattaggctatgccttgattggtggcttagatggttttgatgcatctactagcaatcccaaaaatatcgctgtacaaacagcctgggccaaccaagcacgcgcagaagattcgactggacaaaactactatgaaagtttagttcgaaaggctttggatcaaaataagcgggtgcggtaccgtgtcacattactctacgctacagaagaagatttggttccttctgcatctcagattgaagccaaatcatctgatggtgaattggagtttaacgttgtagttcctaacgttcaaaagggaattcagttagattaccgaacaggaaaagtaactgttacaaagaactaa

SEQ ID NO: 110 - protein, 275 aa, Streptococcus infantis
MNKKTRQALIGLLLFLLLSAGSYYIKEMQASNATPQTQVNQKSQSLDTPSQKLAESVLTDSVKKQIKGTLEWNGSGAFIVNGNKTNLDAKVSSKPYADNKTKTVGGETVPTVANALMSKATRQYKDREETGNGSTSWTPAGWHQVKNLKGTYNHAVDRGHLLGYALIGGLDGFDASTSNPKNIAVQTAWANQARAEDSTGQNYYESLVRKALDQNKRVRYRVTLLYATEEDLVPSASQIEAKSSDGELEFNVVVPNVQKGIQLDYRTGKVTVTKN

SEQ ID NO: 111 - protein, 245 aa, Streptococcus infantis
SNATPQTQVNQKSQSLDTPSQKLAESVLTDSVKKQIKGTLEWNGSGAFIVNGNKTNLDAKVSSKPYADNKTKTVGGETVPTVANALMSKATRQYKDREETGNGSTSWTPAGWHQVKNLKGTYNHAVDRGHLLGYALIGGLDGFDASTSNPKNIAVQTAWANQARAEDSTGQNYYESLVRKALDQNKRVRYRVTLLYATEEDLVPSASQIEAKSSDGELEFNVVVPNVQKGIQLDYRTGKVTVTKN

SEQ ID NO: 112 - skipped

SEQ ID NO: 113 - protein, 5 aa, Synthetic construct
  VARIANT 4: note = X= D or N
PLHXE

SEQ ID NO: 114 - skipped

SEQ ID NO: 115 - protein, 7 aa, Synthetic construct
  VARIANT 7: note = X= A or V
YDRGHQX

SEQ ID NO: 116-117 - skipped (2 entries)

(19) United States
(12) Patent Application Publication
(10) Pub. No.: US 2025/0043264 A1
(43) Pub. Date: Feb. 6, 2025

(54) Polypeptides Having Nuclease Activity

(71) Applicant: Novozymes A/S, Bagsvaerd (DK)

(72) Inventors: Tianqi SUN, Beijing (CN); Jesper Salomon, Holte (DK); Klaus Gori, Dyssegaard (DK); Morten Gjermansen, Greve (DK); Marc Dominique Morant, Frederiksberg (DK); Mary Ann Stringer, Soborg (DK)

(73) Assignee: Novozymes A/S, Bagsvaerd (DK)

(21) Appl. No.: 18/918,723

(22) Filed: Oct. 17, 2024

Related U.S. Application Data
(62) Division of application No. 17/276,436, filed on Mar. 15, 2021.

(30) Foreign Application Priority Data
Oct. 4, 2018 (WO) PCT/CN2018/109243

(51) Int. Cl. C12N 9/22; C11D 3/386

(52) U.S. Cl. CPC C12N 9/22; C11D 3/38636; C11D 2111/12

(57) ABSTRACT

The present invention relates to polypeptides having nuclease activity, and polynucleotides encoding the polypeptides. The invention also relates to nucleic acid constructs, vectors, and recombinant host cells comprising the polynucleotides as well as methods of producing, recovering and using the polypeptides. The invention further relates to cleaning compositions comprising one or more of the polypeptides having nuclease activity, as well as a cleaning method and use of the polypeptides.

### CROSS-REFERENCE TO RELATED APPLICATIONS

This application is a division of U.S. application Ser. No. 17/276,436 filed on Mar. 15, 2021, now pending, which is a 35 U.S.C. 371 national application of international application no. PCT/CN2019/109813 filed Oct. 3, 2019, which claims priority or the benefit under 35 U.S.C. 119 of International application no. PCT/CN2018/109243 filed Oct. 4, 2018. The disclosure of each application is fully incorporated herein by reference.

### REFERENCE TO A SEQUENCE LISTING

This application contains a Sequence Listing in computer readable form, which is incorporated herein by reference. The content of the electronic sequence listing was created on Oct. 17, 2024 and is named SQ.xml and is 178,999 bytes in size.

### FIELD OF THE INVENTION

The present invention relates to polypeptides having nuclease activity, and polynucleotides encoding the polypeptides. The invention also relates to nucleic acid constructs, vectors, and recombinant host cells comprising the polynucleotides as well as methods of producing, recovering and using the polypeptides. The invention further relates to a detergent composition comprising one or more of the polypeptides having nuclease activity. It further concerns a laundering method and the use of the polypeptides having nuclease activity.

### BACKGROUND OF THE INVENTION

When laundry items like T-shirts or sportswear are used, they are exposed to bacteria, body soil, e.g., sweat, dead cells, skin debris, pollution etc. from the body of the user and from the rest of the environment in which they are used. Some of these body soils and pollution may adhere to the laundry item and may form a biofilm on the item. The presence of body soiling implies that the laundry items become sticky and therefore soil adheres to the sticky areas. This soil is difficult to remove using commercially available detergent compositions. Further, when very dirty laundry items are washed together with less dirty laundry items, dirt present in the wash liquor tends to stick to the body soil, such that laundry items can be more “soiled” after wash than before wash. Further, these body soils may be a source of bad odour, which develops after use of the laundry item. The bad odour is difficult to remove and may remain even after wash.

WO 2011/098579 concerns bacterial deoxyribonuclease compounds and methods for biofilm disruption and prevention.

### SUMMARY OF THE INVENTION

The present invention provides polypeptides having nuclease activity; polynucleotides encoding the polypeptides; compositions comprising said polypeptides; methods and use of said polypeptides having nuclease activity.

One aspect of the invention relates to a cleaning composition comprising:
(a) at least 0.001 ppm of a polypeptide have nuclease activity, wherein the polypeptide comprises one or more of the motifs [HQ][FILVY]X[GAQS]DX[HTGSA][QVM]P[LFM]H (SEQ ID NO: 102), G[GA]NX[VILFY]X[VLM](SEQ ID NO: 103) and/or [SADN]R[GS]H (SEQ ID NO: 104); wherein the polypeptide belongs to the Pfam family PF02265 (S1-P1_nuclease), PF01223 (Endonuclease_NS) or PF13930 (Endonuclea_NS_2); and
(b) one or more surfactants.

Another aspect of the invention relates to a polypeptide having nuclease activity, as well as a cleaning composition comprising such as polypeptide, wherein the polypeptide is selected from a first group consisting of:
(a) a polypeptide having at least 99% sequence identity to the polypeptide of SEQ ID NO: 3;
(b) a polypeptide having at least 62% sequence identity to the polypeptide of SEQ ID NO: 6;
(c) a polypeptide having at least 97% sequence identity to the polypeptide of SEQ ID NO: 12; and
(d) a polypeptide having at least 86% sequence identity to the polypeptide of SEQ ID NO: 21 or 27;
(e) a polypeptide having at least 98% sequence identity to the polypeptide of SEQ ID NO: 30;
(f) a polypeptide having 100% sequence identity to the polypeptide of SEQ ID NO: 33 or 48;
(g) a polypeptide having at least 76% sequence identity to the polypeptide of SEQ ID NO: 39 or 51;
(h) a polypeptide having at least 95% sequence identity to the polypeptide of SEQ ID NO: 42;
(i) a polypeptide having at least 79% sequence identity to the polypeptide of SEQ ID NO: 45 or 24;
(j) a polypeptide having at least 94% sequence identity to the polypeptide of SEQ ID NO: 54;
(k) a polypeptide having at least 72% sequence identity to the polypeptide of SEQ ID NO: 57;
(l) a polypeptide having at least 82% sequence identity to the polypeptide of SEQ ID NO: 60;
(m) a polypeptide having at least 75% sequence identity to the polypeptide of SEQ ID NO: 63;
(n) a polypeptide having at least 80% sequence identity to the polypeptide of SEQ ID NO: 69 or 75;
or is selected from a second group consisting of:
(o) a polypeptide having at least 77% sequence identity to the polypeptide of SEQ ID NO: 78;
(p) a polypeptide having at least 94% sequence identity to the polypeptide of SEQ ID NO: 84;
(q) a polypeptide having at least 99% sequence identity to the polypeptide of SEQ ID NO: 87;
(r) a polypeptide having at least 80% sequence identity to the polypeptide of SEQ ID NO: 90; and
(s) a polypeptide having at least 90% sequence identity to the polypeptide of SEQ ID NO: 93;
or is a variant or fragment selected from:
(t) a variant of the polypeptide selected from said first group or said second group wherein the variant has nuclease activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions; and
(u) a fragment of the polypeptide of (a), (b), (c), (d), (e), (f), (g), (h), (i), (j), (k), (I), (m), (n), (o), (p), (q), (r) and (s), wherein said fragment has nuclease activity.

One aspect of the invention relates to a polynucleotide encoding a polypeptide of the invention.

One aspect of the invention relates to a nucleic acid construct or an expression vector comprising a polynucleotide encoding a polypeptide of the invention wherein the polynucleotide is operably linked to one or more control sequences that direct the production of the polypeptide in an expression host.

One aspect of the invention relates to a recombinant host cell comprising a polynucleotide encoding a polypeptide of the invention operably linked to one or more control sequences that direct the production of the polypeptide.

One aspect of the invention relates to a method of producing the polypeptide of the invention, comprising cultivating a cell, which in its wild-type form produces the polypeptide, under conditions conducive for production of the polypeptide, and optionally recovering the polypeptide.

One aspect of the invention relates to a whole broth formulation or cell culture composition comprising a polypeptide of the invention.

One aspect of the invention relates to a cleaning composition comprising:
(a) at least 0.001 ppm of a polypeptide having nuclease activity; and
(b) one or more surfactants;
   wherein the polypeptide is selected from the group consisting of:
(i) a polypeptide having at least 80% sequence identity to the polypeptide of SEQ ID NO: 9;
(ii) a polypeptide having at least 80% sequence identity to the polypeptide of SEQ ID NO: 15;
(iii) a polypeptide having at least 80% sequence identity to the polypeptide of SEQ ID NO: 18;
(iv) a polypeptide having at least 80% sequence identity to the polypeptide of SEQ ID NO: 36;
(v) a polypeptide having at least 80% sequence identity to the polypeptide of SEQ ID NO: 66;
(vi) a polypeptide having at least 80% sequence identity to the polypeptide of SEQ ID NO: 72;
(vii) a polypeptide having at least 80% sequence identity to the polypeptide of SEQ ID NO: 81;
(viii) a polypeptide having at least 80% sequence identity to the polypeptide of SEQ ID NO: 96;
(ix) a polypeptide having at least 80% sequence identity to the polypeptide of SEQ ID NO: 99.

The cleaning compositions herein further preferably comprise, in addition to at least one surfactant, one or more cleaning composition components, preferably selected from surfactants, builders, bleach components, polymers, dispersing agents and additional enzymes.

One aspect relates to a laundering method for laundering an item comprising:
a) exposing an item to a wash liquor comprising a polypeptide of the invention or a cleaning composition comprising a polypeptide of the invention;
b) completing at least one wash cycle; and
c) optionally rinsing the item,
   wherein the item is a textile.

One aspect of the invention relates to the use of a polypeptide according to the invention or a cleaning composition according to the invention for cleaning an item by:
(a) preventing, reducing or removing stickiness of the item;
(b) preventing, reducing or removing biofilm or biofilm components from the item;
(c) preventing, reducing or removing redeposition of soil during cleaning of the item;
(d) preventing, reducing or removing adherence of soil to the item;
(e) maintaining or improving whiteness of the item; or
(f) preventing, reducing or removing malodor from the item,
   wherein the item is a textile.

### Overview of Sequences

S1-P1 Nuclease Genes and their Encoded Polypeptides:
SEQ ID NO: 1: DNA encoding full length polypeptide from Trichoderma hamatum
SEQ ID NO: 2: polypeptide derived from SEQ ID NO: 1
SEQ ID NO: 3: mature polypeptide obtained from Trichoderma hamatum
SEQ ID NO: 4: DNA encoding full length polypeptide from Morchella costata
SEQ ID NO: 5: polypeptide derived from SEQ ID NO: 4
SEQ ID NO: 6: mature polypeptide obtained from Morchella costata
SEQ ID NO: 7: DNA encoding full length polypeptide from Trichoderma reesei
SEQ ID NO: 8: polypeptide derived from SEQ ID NO: 7
SEQ ID NO: 9: mature polypeptide obtained from Trichoderma reesei
SEQ ID NO: 10: DNA encoding full length polypeptide from Penicillium cremeogriseum
SEQ ID NO: 11: polypeptide derived from SEQ ID NO: 10
SEQ ID NO: 12: mature polypeptide obtained from Penicillium cremeogriseum
SEQ ID NO: 13: DNA encoding full length polypeptide from Stenocarpella maydis
SEQ ID NO: 14: polypeptide derived from SEQ ID NO: 13
SEQ ID NO: 15: mature polypeptide obtained from Stenocarpella maydis
SEQ ID NO: 16: DNA encoding full length polypeptide from Stenocarpella maydis
SEQ ID NO: 17: polypeptide derived from SEQ ID NO: 16
SEQ ID NO: 18: mature polypeptide obtained from Stenocarpella maydis
SEQ ID NO: 19: DNA encoding full length polypeptide from Cordyceps cardinalis
SEQ ID NO: 20: polypeptide derived from SEQ ID NO: 19
SEQ ID NO: 21: mature polypeptide obtained from Cordyceps cardinalis
SEQ ID NO: 22: DNA encoding full length polypeptide from Phialophora geniculata
SEQ ID NO: 23: polypeptide derived from SEQ ID NO: 22
SEQ ID NO: 24: mature polypeptide obtained from Phialophora geniculata
SEQ ID NO: 25: DNA encoding full length polypeptide from Cadophora fastigiata
SEQ ID NO: 26: polypeptide derived from SEQ ID NO: 25
SEQ ID NO: 27: mature polypeptide obtained from Cadophora fastigiata
SEQ ID NO: 28: DNA encoding full length polypeptide from Microbial enrichment A
SEQ ID NO: 29: polypeptide derived from SEQ ID NO: 28
SEQ ID NO: 30: mature polypeptide obtained from Microbial enrichment A.
SEQ ID NO: 31: DNA encoding full length polypeptide from Lysobacter enzymogenes
SEQ ID NO: 32: polypeptide derived from SEQ ID NO: 31
SEQ ID NO: 33: mature polypeptide obtained from Lysobacter enzymogenes
SEQ ID NO: 34: DNA encoding full length polypeptide from Pseudoalteromonas nigrifaciens
SEQ ID NO: 35: polypeptide derived from SEQ ID NO: 34
SEQ ID NO: 36: mature polypeptide obtained from Pseudoalteromonas nigrifaciens
SEQ ID NO: 37: DNA encoding full length polypeptide from Vibrio sp.
SEQ ID NO: 38: polypeptide derived from SEQ ID NO: 37
SEQ ID NO: 39: mature polypeptide obtained from Vibrio sp.
SEQ ID NO: 40: DNA encoding full length polypeptide from Janthinobacterium agaricidamnosum
SEQ ID NO: 41: polypeptide derived from SEQ ID NO: 40
SEQ ID NO: 42: mature polypeptide obtained from Janthinobacterium agaricidamnosum
SEQ ID NO: 43: DNA encoding full length polypeptide from Massilia aerilata
SEQ ID NO: 44: polypeptide derived from SEQ ID NO: 43
SEQ ID NO: 45: mature polypeptide obtained from Massilia aerilata
SEQ ID NO: 46: DNA encoding full length polypeptide from Aspergillus oryzae
SEQ ID NO: 47: polypeptide derived from SEQ ID NO: 46
SEQ ID NO: 48: mature polypeptide obtained from Aspergillus oryzae
SEQ ID NO: 49: DNA encoding full length polypeptide from Penicillium atramentosum
SEQ ID NO: 50: polypeptide derived from SEQ ID NO: 49
SEQ ID NO: 51: mature polypeptide obtained from Penicillium atramentosum
SEQ ID NO: 52: DNA encoding full length polypeptide from Penicillium emersonii
SEQ ID NO: 53: polypeptide derived from SEQ ID NO: 52
SEQ ID NO: 54: mature polypeptide obtained from Penicillium emersonii
SEQ ID NO: 55: DNA encoding full length polypeptide from Ostropa barbara
SEQ ID NO: 56: polypeptide derived from SEQ ID NO: 55
SEQ ID NO: 57: mature polypeptide obtained from Ostropa barbara
SEQ ID NO: 58: DNA encoding full length polypeptide from Pyrenochaetopsis sp.
SEQ ID NO: 59: polypeptide derived from SEQ ID NO: 58
SEQ ID NO: 60: mature polypeptide obtained from Pyrenochaetopsis sp.
SEQ ID NO: 61: DNA encoding full length polypeptide from Lachnellula sp.
SEQ ID NO: 62: polypeptide derived from SEQ ID NO: 61
SEQ ID NO: 63: mature polypeptide obtained from Lachnellula sp.
SEQ ID NO: 64: DNA encoding full length polypeptide from Trichoderma reesei
SEQ ID NO: 65: polypeptide derived from SEQ ID NO: 64
SEQ ID NO: 66: mature polypeptide obtained from Trichoderma reesei
SEQ ID NO: 67: DNA encoding full length polypeptide from Cordyceps cardinalis
SEQ ID NO: 68: polypeptide derived from SEQ ID NO: 67
SEQ ID NO: 69: mature polypeptide obtained from Cordyceps cardinalis
SEQ ID NO: 70: DNA encoding full length polypeptide from Acremonium alcalophilum
SEQ ID NO: 71: polypeptide derived from SEQ ID NO: 70
SEQ ID NO: 72: mature polypeptide obtained from Acremonium alcalophilum
SEQ ID NO: 73: DNA encoding full length polypeptide from Microdochium phragmitis
SEQ ID NO: 74: polypeptide derived from SEQ ID NO: 73
SEQ ID NO: 75: mature polypeptide obtained from Microdochium phragmitis
   EN_NS Nuclease Genes and their Encoded Polypeptides:
SEQ ID NO: 76: DNA encoding full length polypeptide from Bacillus deramificans
SEQ ID NO: 77: polypeptide derived from SEQ ID NO: 76
SEQ ID NO: 78: mature polypeptide obtained from Bacillus deramificans
SEQ ID NO: 79: DNA encoding full length polypeptide from Bacillus thuringiensis
SEQ ID NO: 80: polypeptide derived from SEQ ID NO: 79
SEQ ID NO: 81: mature polypeptide obtained from Bacillus thuringiensis
SEQ ID NO: 82: DNA encoding full length polypeptide from Penicillium virgatum
SEQ ID NO: 83: polypeptide derived from SEQ ID NO: 82
SEQ ID NO: 84: mature polypeptide obtained from Penicillium virgatum
SEQ ID NO: 85: DNA encoding full length polypeptide from Streptomyces cirratus
SEQ ID NO: 86: polypeptide derived from SEQ ID NO: 85
SEQ ID NO: 87: mature polypeptide obtained from Streptomyces cirratus
SEQ ID NO: 88: DNA encoding full length polypeptide from Acremonium sp. XZ1968
SEQ ID NO: 89: polypeptide derived from SEQ ID NO: 88
SEQ ID NO: 90: mature polypeptide obtained from Acremonium sp. XZ1968
SEQ ID NO: 91: DNA encoding full length polypeptide from Daldinia fissa
SEQ ID NO: 92: polypeptide derived from SEQ ID NO: 91
SEQ ID NO: 93: mature polypeptide obtained from Daldinia fissa
SEQ ID NO: 94: DNA encoding full length polypeptide from Actinomucor elegans
SEQ ID NO: 95: polypeptide derived from SEQ ID NO: 94
SEQ ID NO: 96: mature polypeptide obtained from Actinomucor elegans
SEQ ID NO: 97: DNA encoding full length polypeptide from Talaromyces leycettanus
SEQ ID NO: 98: polypeptide derived from SEQ ID NO: 97
SEQ ID NO: 99: mature polypeptide obtained from Talaromyces leycettanus

### Cloning Primers:

SEQ ID NO: 100:
5′-GACGCGGCCGCACCATGCCGCGCTTACTCCC
SEQ ID NO: 101:
5′-GACGCGATCGCTCAAGAGGGCTGACTCG

### Motifs:

SEQ ID NO: 102:
[HQ][FILVY]X[GAQS]DX[HIGSA][QVM]P[LFM]H
SEQ ID NO: 103:
G[GA]NX[VILFY]X[VLM]
SEQ ID NO: 104:
[SADN]R[GS]H

### Signal Peptide:

SEQ ID NO: 105: MKKPLGKIVASTALLISVAFSSSIASA
   Additional EN_NS Nuclease Genes and their Encoded Polypeptides:
SEQ ID NO: 106: DNA encoding full length polypeptide from Bacillus sp.
SEQ ID NO: 107: polypeptide derived from SEQ ID NO: 106 SEQ ID NO: 108: mature polypeptide obtained from Bacillus sp.
SEQ ID NO: 109: DNA encoding full length polypeptide from Streptococcus infantis
SEQ ID NO: 110: polypeptide derived from SEQ ID NO: 109
SEQ ID NO: 111: mature polypeptide obtained from Streptococcus infantis

### Additional Motifs:

SEQ ID NO: 112:
P[LM]H[VA][GA]
SEQ ID NO: 113:
PLH[DN]E
SEQ ID NO: 114:
[YV][DN]RGH
SEQ ID NO: 115:
YDRGHQ[AV]
SEQ ID NO: 116:
[DNA]R[GSC]H[LI]
SEQ ID NO: 117:
[RIENLG][YF][RHN]V

### Definitions

The term “nuclease” means a polypeptide having nuclease activity, and in the context of the present invention is used in relation to two groups of polypeptides. The first group of polypeptides are S1-P1 nucleases that cleave single-stranded DNA and RNA with no sequence specificity (EC:3.1.30.1), and may also introduce single-stranded breaks in double-stranded DNA or RNA, or DNA-RNA hybrids. Polypeptides of this first group comprise an S1-P1_nuclease domain (Pfam domain id PF02265, Pfam version 31.0 Finn (2016). Nucleic Acids Research, Database Issue 44:D279-D285).

The second group of polypeptides are classified as exonucleases or endonucleases (EN_NS nucleases), since they are characterized by having a functional Endonuclease_NS or an Endonuclea_NS_2 domain, identified as Pfam domain id PF01223 or PF13930 (Finn et al., (2016) Nucleic Acids Research, 44:D279-D285 and Pfam version 31.0). The Endonuclease_NS and the Endonuclea_NS_2 are functional domains providing catalytic activity to the polypeptide.

S1_P1-nucleases are non-specific nucleases that cleave DNA or RNA in single-stranded or double-stranded nucleic acids.

Endonuclease_NS nucleases are non-specific endo-nucleases that cleave DNA and RNA, cleaving double-stranded or single-stranded nucleic acids.

For purposes of the present invention, nuclease activity is determined according to the procedure described in the Examples. The first group of polypeptides of the present invention have at least 20%, e.g., at least 40%, at least 50%, at least 60%, at least 70%, at least 80%, at least 90%, at least 95%, or at least 100% of the nuclease activity of the mature polypeptide of SEQ ID NO: 3, 6, 9, 12, 15, 18, 21, 24, 27, 30, 33, 36, 39, 42, 45, 48, 51, 54, 57, 60, 63, 66, 69, 72 or 75,

The second group of polypeptides of the present invention have at least 20%, e.g., at least 40%, at least 50%, at least 60%, at least 70%, at least 80%, at least 90%, at least 95%, or at least 100% of the nuclease activity of the mature polypeptide of SEQ ID NO: 78, 81, 84, 87, 90, 93, 96, 99, 108 or 111.

The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

The term “biofilm” means a film produced by any group of microorganisms in which cells stick to each other or stick to a surface, such as a textile, dishware or hard surface or another kind of surface. These adherent cells are frequently embedded within a self-produced matrix of extracellular polymeric substance (EPS). Biofilm EPS is a polymeric conglomeration generally composed of extracellular DNA, proteins, and polysaccharides. Biofilms may form on living or non-living surfaces. The microbial cells growing in a biofilm are physiologically distinct from planktonic cells of the same organism, which, by contrast, are single-cells that may float or swim in a liquid medium. Bacteria living in a biofilm usually have significantly different properties from planktonic bacteria of the same species, as the dense and protected environment of the film allows them to cooperate and interact in various ways. One benefit of this environment for the microorganisms is increased resistance to detergents and antibiotics, as the dense extracellular matrix and the outer layer of cells protect the interior of the community. On laundry biofilm producing bacteria can be found among species including Acinetobacter sp., Aeromicrobium sp., Brevundimonas sp., Microbacterium sp., Micrococcus luteus, Pseudomonas sp., Staphylococcus epidermidis, and Stenotrophomonas sp. On hard surfaces biofilm producing bacteria can be found among the following species: Acinetobacter sp., Aeromicrobium sp., Brevundimonas sp., Microbacterium sp., Micrococcus luteus, Pseudomonas sp., Staphylococcus epidermidis, Staphylococcus aureus and Stenotrophomonas sp.

The term “catalytic domain” means the region of an enzyme containing the catalytic machinery of the enzyme.

The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

The term “deep cleaning” means in this context disruption, reduction or removal of organic components such as polysaccharides, proteins, RNA, DNA, soil or other components present in organic matter such as biofilm.

The term “cleaning composition” includes “detergent composition” and refers to compositions that find use in the removal of undesired compounds from items to be cleaned, such as textiles, dishware and hard surfaces. The detergent composition may be used to, e.g., clean textiles for both household cleaning and industrial cleaning. The terms encompass any materials/compounds selected for the particular type of cleaning composition desired and the form of the product (e.g., liquid, gel, powder, granulate, paste, or spray compositions) and include, but are not limited to, detergent compositions (e.g., liquid and/or solid laundry detergents and fine fabric detergents; fabric fresheners; fabric softeners; and textile and laundry pre-spotters/pretreatment).

In addition to containing the enzyme of the invention, the cleaning composition may contain one or more additional enzymes (such as DNases, proteases, amylases, lipases, cutinases, cellulases, endoglucanases, xyloglucanases, pectinases, pectin lyases, xanthanases, peroxidases, haloperoxygenases, catalases, galactanase, mannanases, or any mixture thereof), and/or cleaning components such as surfactants, builders, chelators or chelating agents, bleach system or bleach components, polymers, foam boosters, suds suppressors, dyes, perfume, tannish inhibitors, optical brighteners, bactericides, fungicides, soil suspending agents, anti-corrosion agents, enzyme inhibitors or stabilizers, enzyme activators, transferase(s), hydrolytic enzymes, oxido reductases, bluing agents and fluorescent dyes, antioxidants, and solubilizers.

The term “expression” includes any step involved in the production of a polypeptide including, but not limited to, transcription, post-transcriptional modification, translation, post-translational modification, and secretion.

The term “expression vector” means a linear or circular DNA molecule that comprises a polynucleotide encoding a polypeptide and is operably linked to control sequences that provide for its expression.

The term “fragment” means a polypeptide or a catalytic domain having one or more amino acids absent from the amino and/or carboxyl terminus of a mature polypeptide or domain; wherein the fragment has nuclease activity.

The term “His-tag” refers to a polyhistidine tag typically comprising at least 6 histidine residues, that may be added to the N- or C-terminal. His-tags are known in the art for use in, e.g., protein purification, but may also be used for improving solubility at low pH values. Similarly, an “HQ-tag”, i.e., a histidine-glutamine tag, may also be used for the purpose of purification as is known in the art.

The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

The term “improved wash performance” is defined herein as an enzyme displaying an increased wash performance in a detergent composition relative to the wash performance of same detergent composition without the enzyme, e.g., by increased stain removal or less redeposition. The term “improved wash performance” includes wash performance in laundry, hard surface and dish washing.

The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., recombinant production in a host cell; multiple copies of a gene encoding the substance; and use of a stronger promoter than the promoter naturally associated with the gene encoding the substance). An isolated substance may be present in a fermentation broth sample; e.g., a host cell may be genetically modified to express the polypeptide of the invention. The fermentation broth from that host cell will comprise the isolated polypeptide.

The term “laundering” relates to both household laundering and industrial laundering and means the process of treating textiles with a solution containing a cleaning or detergent composition of the present invention. The laundering process can for example be carried out using, e.g., a household or an industrial washing machine or can be carried out by hand.

The term “malodor” means an odor which is not desired on clean items. The cleaned item should smell fresh and clean without malodors adhered to the item. One example of malodor is compounds with an unpleasant smell which may be produced by microorganisms and trapped within a biofilm or stick to the “glue” of a biofilm. Other examples of unpleasant smells are sweat or body odor adhered to an item which has been in contact with a human or animal. Other examples of malodor are odors from spices which stick to items, for example curry or other exotic spices with a strong smell.

The term “mature polypeptide” means a polypeptide in its mature form following N-terminal processing (e.g., removal of signal peptide).

In one aspect, the mature polypeptide is amino acids 1 to 302 of SEQ ID NO: 2. Amino acids −19 to −1 of SEQ ID NO: 2 is the signal peptide.

In one aspect, the mature polypeptide is amino acids 1 to 336 of SEQ ID NO: 5. Amino acids −21 to −1 of SEQ ID NO: 5 is the signal peptide.

In one aspect, the mature polypeptide is amino acids 1 to 324 of SEQ ID NO: 8. Amino acids −20 to −1 of SEQ ID NO: 8 is the signal peptide.

In one aspect, the mature polypeptide is amino acids 1 to 322 of SEQ ID NO: 11. Amino acids −20 to −1 of SEQ ID NO: 11 is the signal peptide.

In one aspect, the mature polypeptide is amino acids 1 to 276 of SEQ ID NO: 14. Amino acids −19 to −1 of SEQ ID NO: 14 is the signal peptide.

In one aspect, the mature polypeptide is amino acids 1 to 321 of SEQ ID NO: 17. Amino acids −17 to −1 of SEQ ID NO: 17 is the signal peptide.

In one aspect, the mature polypeptide is amino acids 1 to 314 of SEQ ID NO: 20. Amino acids −20 to −1 of SEQ ID NO: 20 is the signal peptide.

In one aspect, the mature polypeptide is amino acids 1 to 302 of SEQ ID NO: 23. Amino acids −19 to −1 of SEQ ID NO: 23 is the signal peptide.

In one aspect, the mature polypeptide is amino acids 1 to 273 of SEQ ID NO: 26. Amino acids −20 to −1 of SEQ ID NO: 26 is the signal peptide.

In one aspect, the mature polypeptide is amino acids 1 to 240 of SEQ ID NO: 29. Amino acids −30 to −1 of SEQ ID NO: 29 is the signal peptide.

In one aspect, the mature polypeptide is amino acids 1 to 252 of SEQ ID NO: 32. Amino acids −34 to −1 of SEQ ID NO: 32 is the signal peptide.

In one aspect, the mature polypeptide is amino acids 1 to 258 of SEQ ID NO: 35. Amino acids −26 to −1 of SEQ ID NO: 35 is the signal peptide.

In one aspect, the mature polypeptide is amino acids 1 to 251 of SEQ ID NO: 38. Amino acids −24 to −1 of SEQ ID NO: 38 is the signal peptide.

In one aspect, the mature polypeptide is amino acids 1 to 324 of SEQ ID NO: 41. Amino acids −21 to −1 of SEQ ID NO: 41 is the signal peptide.

In one aspect, the mature polypeptide is amino acids 1 to 332 of SEQ ID NO: 44. Amino acids −2 to −1 of SEQ ID NO: 44 is the signal peptide.

In one aspect, the mature polypeptide is amino acids 1 to 322 of SEQ ID NO: 47. Amino acids −20 to −1 of SEQ ID NO: 47 is the signal peptide.

In one aspect, the mature polypeptide is amino acids 1 to 324 of SEQ ID NO: 50. Amino acids −19 to −1 of SEQ ID NO: 50 is the signal peptide.

In one aspect, the mature polypeptide is amino acids 1 to 267 of SEQ ID NO: 53. Amino acids −19 to −1 of SEQ ID NO: 53 is the signal peptide.

In one aspect, the mature polypeptide is amino acids 1 to 271 of SEQ ID NO: 56. Amino acids −19 to −1 of SEQ ID NO: 56 is the signal peptide.

In one aspect, the mature polypeptide is amino acids 1 to 292 of SEQ ID NO: 59. Amino acids −18 to −1 of SEQ ID NO: 59 is the signal peptide.

In one aspect, the mature polypeptide is amino acids 1 to 270 of SEQ ID NO: 62. Amino acids −21 to −1 of SEQ ID NO: 62 is the signal peptide.

In one aspect, the mature polypeptide is amino acids 1 to 308 of SEQ ID NO: 65. Amino acids −19 to −1 of SEQ ID NO: 65 is the signal peptide.

In one aspect, the mature polypeptide is amino acids 1 to 288 of SEQ ID NO: 68. Amino acids −19 to −1 of SEQ ID NO: 68 is the signal peptide.

In one aspect, the mature polypeptide is amino acids 1 to 285 of SEQ ID NO: 71. Amino acids −19 to −1 of SEQ ID NO: 71 is the signal peptide.

In one aspect, the mature polypeptide is amino acids 1 to 308 of SEQ ID NO: 74. Amino acids −20 to −1 of SEQ ID NO: 74 is the signal peptide.

In one aspect, the mature polypeptide is amino acids 1 to 371 of SEQ ID NO: 77. Amino acids −28 to −1 of SEQ ID NO: 77 is the signal peptide.

In one aspect, the mature polypeptide is amino acids 1 to 235 of SEQ ID NO: 80. Amino acids −25 to −1 of SEQ ID NO: 80 is the signal peptide.

In one aspect, the mature polypeptide is amino acids 1 to 302 of SEQ ID NO: 83. Amino acids −22 to −1 of SEQ ID NO: 83 is the signal peptide.

In one aspect, the mature polypeptide is amino acids 1 to 246 of SEQ ID NO: 86. Amino acids −28 to −1 of SEQ ID NO: 86 is the signal peptide.

In one aspect, the mature polypeptide is amino acids 1 to 322 of SEQ ID NO: 89. Amino acids −21 to −1 of SEQ ID NO: 89 is the signal peptide.

In one aspect, the mature polypeptide is amino acids 1 to 345 of SEQ ID NO: 92. Amino acids −18 to −1 of SEQ ID NO: 92 is the signal peptide.

In one aspect, the mature polypeptide is amino acids 1 to 279 of SEQ ID NO: 95. Amino acids −25 to −1 of SEQ ID NO: 95 is the signal peptide.

In one aspect, the mature polypeptide is amino acids 1 to 319 of SEQ ID NO: 98. Amino acids −17 to −1 of SEQ ID NO: 98 is the signal peptide.

It is known in the art that a host cell may produce a mixture of two of more different mature polypeptides (i.e., with a different C-terminal and/or N-terminal amino acid) expressed by the same polynucleotide. It is also known in the art that different host cells process polypeptides differently, and thus, one host cell expressing a polynucleotide may produce a different mature polypeptide (e.g., having a different C-terminal and/or N-terminal amino acid) as compared to another host cell expressing the same polynucleotide.

The term “mature polypeptide coding sequence” means a polynucleotide that encodes a mature polypeptide having nuclease activity. In one aspect, the mature polypeptide coding sequence is nucleotides 58 to 1095 of SEQ ID NO: 1; and nucleotides 1 to 57 of SEQ ID NO: 1 encode a signal peptide.

In another aspect, the mature polypeptide coding sequence is nucleotides 64 to 1135 of SEQ ID NO: 4; and nucleotides 1 to 63 of SEQ ID NO: 4 encode a signal peptide.

In another aspect, the mature polypeptide coding sequence is nucleotides 61 to 1113 of SEQ ID NO: 7; and nucleotides 1 to 60 of SEQ ID NO: 7 encode a signal peptide. In another aspect, the mature polypeptide coding sequence is nucleotides 61 to 1078 of SEQ ID NO: 10; and nucleotides 1 to 60 of SEQ ID NO: 10 encode a signal peptide.

In another aspect, the mature polypeptide coding sequence is nucleotides 58 to 1124 of SEQ ID NO: 13; and nucleotides 1 to 57 of SEQ ID NO: 13 encode a signal peptide.

In another aspect, the mature polypeptide coding sequence is nucleotides 52 to 1083 of SEQ ID NO: 16; and nucleotides 1 to 51 of SEQ ID NO: 16 encode a signal peptide.

In another aspect, the mature polypeptide coding sequence is nucleotides 61 to 1119 of SEQ ID NO: 19; and nucleotides 1 to 60 of SEQ ID NO: 19 encode a signal peptide.

In another aspect, the mature polypeptide coding sequence is nucleotides 58 to 634 of SEQ ID NO: 22; and nucleotides 1 to 57 of SEQ ID NO: 22 encode a signal peptide.

In another aspect, the mature polypeptide coding sequence is nucleotides 61 to 1165 of SEQ ID NO: 25; and nucleotides 1 to 60 of SEQ ID NO: 25 encode a signal peptide.

In another aspect, the mature polypeptide coding sequence is nucleotides 91 to 810 of SEQ ID NO: 28; and nucleotides 1 to 90 of SEQ ID NO: 28 encode a signal peptide.

In another aspect, the mature polypeptide coding sequence is nucleotides 103 to 858 of SEQ ID NO: 31; and nucleotides 1 to 102 of SEQ ID NO: 31 encode a signal peptide.

In another aspect, the mature polypeptide coding sequence is nucleotides 79 to 852 of SEQ ID NO: 34; and nucleotides 1 to 78 of SEQ ID NO: 34 encode a signal peptide.

In another aspect, the mature polypeptide coding sequence is nucleotides 70 to 822 of SEQ ID NO: 37; and nucleotides 1 to 69 of SEQ ID NO: 37 encode a signal peptide.

In another aspect, the mature polypeptide coding sequence is nucleotides 64 to 1035 of SEQ ID NO: 40; and nucleotides 1 to 57 of SEQ ID NO: 40 encode a signal peptide.

In another aspect, the mature polypeptide coding sequence is nucleotides 64 to 1059 of SEQ ID NO: 43; and nucleotides 1 to 63 of SEQ ID NO: 43 encode a signal peptide.

In another aspect, the mature polypeptide coding sequence is nucleotides 61 to 960 of SEQ ID NO: 46; and nucleotides 1 to 60 of SEQ ID NO: 46 encode a signal peptide.

In another aspect, the mature polypeptide coding sequence is nucleotides 58 to 1099 of SEQ ID NO: 49; and nucleotides 1 to 57 of SEQ ID NO: 49 encode a signal peptide.

In another aspect, the mature polypeptide coding sequence is nucleotides 58 to 952 of SEQ ID NO: 52; and nucleotides 1 to 57 of SEQ ID NO: 52 encode a signal peptide.

In another aspect, the mature polypeptide coding sequence is nucleotides 58 to 1083 of SEQ ID NO: 55; and nucleotides 1 to 57 of SEQ ID NO: 55 encode a signal peptide.

In another aspect, the mature polypeptide coding sequence is nucleotides 55 to 1044 of SEQ ID NO: 58; and nucleotides 1 to 54 of SEQ ID NO: 58 encode a signal peptide.

In another aspect, the mature polypeptide coding sequence is nucleotides 64 to 1445 of SEQ ID NO: 61; and nucleotides 1 to 63 of SEQ ID NO: 61 encode a signal peptide.

In another aspect, the mature polypeptide coding sequence is nucleotides 58 to 1116 of SEQ ID NO: 57; and nucleotides 1 to 57 of SEQ ID NO: 64 encode a signal peptide.

In another aspect, the mature polypeptide coding sequence is nucleotides 58 to 1064 of SEQ ID NO: 67; and nucleotides 1 to 57 of SEQ ID NO: 67 encode a signal peptide.

In another aspect, the mature polypeptide coding sequence is nucleotides 58 to 1100 of SEQ ID NO: 70; and nucleotides 1 to 57 of SEQ ID NO: 70 encode a signal peptide.

In another aspect, the mature polypeptide coding sequence is nucleotides 61 to 1054 of SEQ ID NO: 73; and nucleotides 1 to 60 of SEQ ID NO: 73 encode a signal peptide.

In another aspect, the mature polypeptide coding sequence is nucleotides 85 to 1197 of SEQ ID NO: 76; and nucleotides 1 to 84 of SEQ ID NO: 76 encode a signal peptide.

In another aspect, the mature polypeptide coding sequence is nucleotides 76 to 780 of SEQ ID NO: 79; and nucleotides 1 to 75 of SEQ ID NO: 79 encode a signal peptide.

In another aspect, the mature polypeptide coding sequence is nucleotides 67 to 972 of SEQ ID NO: 82; and nucleotides 1 to 66 of SEQ ID NO: 82 encode a signal peptide.

In another aspect, the mature polypeptide coding sequence is nucleotides 85 to 822 of SEQ ID NO: 85; and nucleotides 1 to 84 of SEQ ID NO: 85 encode a signal peptide.

In another aspect, the mature polypeptide coding sequence is nucleotides 64 to 1029 of SEQ ID NO: 88; and nucleotides 1 to 63 of SEQ ID NO: 88 encode a signal peptide.

In another aspect, the mature polypeptide coding sequence is nucleotides 55 to 1089 of SEQ ID NO: 91; and nucleotides 1 to 75 of SEQ ID NO: 91 encode a signal peptide.

In another aspect, the mature polypeptide coding sequence is nucleotides 76 to 973 of SEQ ID NO: 94; and nucleotides 1 to 75 of SEQ ID NO: 94 encode a signal peptide.

In another aspect, the mature polypeptide coding sequence is nucleotides 52 to 1008 of SEQ ID NO: 97; and nucleotides 1 to 51 of SEQ ID NO: 97 encode a signal peptide.

The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

The term “operably linked” means a configuration in which a control sequence is placed at an appropriate position relative to the coding sequence of a polynucleotide such that the control sequence directs expression of the coding sequence. The relatedness between two amino acid sequences or between two nucleotide sequences is described by the parameter “sequence identity.

For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment)

The term “subsequence” means a polynucleotide having one or more nucleotides absent from the 5′ and/or 3′ end of a mature polypeptide coding sequence; wherein the subsequence encodes a fragment having nuclease activity.

The term “variant” means a polypeptide having nuclease activity comprising an alteration, i.e., a substitution, insertion, and/or deletion, at one or more positions. A substitution means replacement of the amino acid occupying a position with a different amino acid; a deletion means removal of the amino acid occupying a position; and an insertion means adding an amino acid adjacent to and immediately following the amino acid occupying a position The term “textile” means any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose-based such as natural cellulosics, including cotton, flax/linen, jute, ramie, sisal or coir or manmade cellulosics (e.g., originating from wood pulp) including viscose/rayon, cellulose acetate fibers (tricell), lyocell or blends thereof. The textile or fabric may also be non-cellulose-based such as natural polyamides including wool, camel, cashmere, mohair, rabbit and silk or synthetic polymers such as nylon, aramid, polyester, acrylic, polypropylene and spandex/elastane, or blends thereof as well as blends of cellulose based and non-cellulose based fibers. Examples of blends are blends of cotton and/or rayon/viscose with one or more companion material such as wool, synthetic fiber (e.g., polyamide fiber, acrylic fiber, polyester fiber, polyvinyl chloride fiber, polyurethane fiber, polyurea fiber, aramid fiber), and/or cellulose-containing fiber (e.g., rayon/viscose, ramie, flax/linen, jute, cellulose acetate fiber, lyocell). Fabric may be conventional washable laundry, for example stained household laundry. When the term fabric or garment is used it is intended to include the broader term textiles as well.

The term “wash cycle” is defined herein as a washing operation wherein one or more items are exposed to a wash liquor, and the items and wash liquor are subjected to interaction, e.g., by applying is mechanical action of some kind to the wash liquor and/or to the item, or by spraying the washing liquor on to the items, in order to release stains and to facilitate flow of wash liquor in or around the items, and finally the superfluous wash liquor is removed. After one or more wash cycles, the items are generally rinsed and dried. The term “wash liquor” is defined herein as the solution or mixture of water and a cleaning composition.

Nomenclature: For purposes of the present invention, the nomenclature [E/Q] or simply [EQ] means that the amino acid at this position may be a glutamic acid (Glu, E) or a glutamine (Gln, Q). Likewise, the nomenclature [V/G/A/I] or [VGAI] means that the amino acid at this position may be a valine (Val, V), glycine (Gly, G), alanine (Ala, A) or isoleucine (Ile, I), and so forth for other combinations as described herein. Unless otherwise limited further, the amino acid X is defined such that it may be any of the 20 natural amino acids.

### DETAILED DESCRIPTION OF THE INVENTION

### Cleaning Compositions and Polypeptides Having Nuclease Activity

The present invention provides polypeptides having nuclease activity, whose members are characterized by having a functional nuclease domain conferring the catalytic activity of the polypeptide, as well as cleaning compositions comprising the polypeptides.

According to a first embodiment, a polypeptide having nuclease activity is an S1-P1 nuclease. S1-P1 nucleases cleave single stranded DNA and RNA with no sequence specificity, and may also introduce single-stranded breaks in double-stranded DNA or RNA, or DNA-RNA hybrids. The S1-P1 comprises an S1-P1_nuclease domain (Pfam domain id PF02265, Pfam version 31.0 Finn (2016). Nucleic Acids Research, Database Issue 44:D279-D285) which is a functional domain conferring hydrolytic activity to the polypeptide, classified as EC:3.1.30.1. In nature S1-P1 nucleases are secreted proteins.

According to a second embodiment, the polypeptides having nuclease activity are classified as exonucleases or endonucleases, since they are characterized by having a functional Endonuclease_NS or an Endonuclea_NS_2 domain, identified as Pfam domain id PF01223 and PF13930 (Finn et al., (2016) Nucleic Acids Research, 44:D279-D285 and Pfam version 31.0). The Endonuclease_NS and the Endonuclea_NS_2 is a functional domain providing catalytic activity to the polypeptide.

In one embodiment, the polypeptide is an S1-P1 nuclease that belongs to the Pfam family PF02265 (S1-P1_nuclease), and which comprises the motif [HQ][FILVY]X[GAQS]DX[HTGSA][QVM]P[LFM]H (SEQ ID NO: 102) and/or G[GA]NX[VILFY]X[VLM](SEQ ID NO: 103).

In a particular embodiment, the polypeptide is an S1-P1 nuclease of bacterial origin and comprises the motif P[LM]H[VA][GA](SEQ ID NO: 112).

In another particular embodiment, the polypeptide is an S1-P1 nuclease of fungal origin and comprises the motif PLH[DN]E (SEQ ID NO: 113).

In another embodiment, the polypeptide is an EN_NS nuclease that belongs to the Pfam family PF01223 (Endonuclease_NS) or PF13930 (Endonuclea_NS_2), and which comprises the motif [SADN]R[GS]H (SEQ ID NO: 104) and/or [YV][DN]RGH (SEQ ID NO: 114), preferably [YV][DN]RGH (SEQ ID NO: 114).

In one particular embodiment, the polypeptide is an EN_NS nuclease of fungal origin, in particular one that belongs to the Pfam family PF01223 (Endonuclease_NS), and which comprises the motif YDRGHQ[AV](SEQ ID NO: 115).

In another particular embodiment, the polypeptide is an EN_NS nuclease of fungal origin, in particular one that belongs to the Pfam family PF01223 (Endonuclease_NS), and which comprises the motif [DNA]R[GSC]H[LI](SEQ ID NO: 116) and/or [RIENLG][YF][RHN]V (SEQ ID NO: 117).

In a first aspect the invention concerns a polypeptide having nuclease activity, selected from a first group consisting of:
(a) a polypeptide having at least 99% sequence identity to the polypeptide of SEQ ID NO: 3;
(b) a polypeptide having at least 62% sequence identity to the polypeptide of SEQ ID NO: 6;
(c) a polypeptide having at least 97% sequence identity to the polypeptide of SEQ ID NO: 12; and
(d) a polypeptide having at least 86% sequence identity to the polypeptide of SEQ ID NO: 21 or 27;
(e) a polypeptide having at least 98% sequence identity to the polypeptide of SEQ ID NO: 30;
(f) a polypeptide having 100% sequence identity to the polypeptide of SEQ ID NO: 33 or 48;
(g) a polypeptide having at least 76% sequence identity to the polypeptide of SEQ ID NO: 39 or 51;
(h) a polypeptide having at least 95% sequence identity to the polypeptide of SEQ ID NO: 42;
(i) a polypeptide having at least 79% sequence identity to the polypeptide of SEQ ID NO: 45 or 24;
(j) a polypeptide having at least 94% sequence identity to the polypeptide of SEQ ID NO: 54;
(k) a polypeptide having at least 72% sequence identity to the polypeptide of SEQ ID NO: 57;
(l) a polypeptide having at least 82% sequence identity to the polypeptide of SEQ ID NO: 60;
(m) a polypeptide having at least 75% sequence identity to the polypeptide of SEQ ID NO: 63;
(n) a polypeptide having at least 80% sequence identity to the polypeptide of SEQ ID NO: 69 or 75;
or selected from a second group consisting of:
(o) a polypeptide having at least 77% sequence identity to the polypeptide of SEQ ID NO: 78;
(p) a polypeptide having at least 94% sequence identity to the polypeptide of SEQ ID NO: 84;
(q) a polypeptide having at least 99% sequence identity to the polypeptide of SEQ ID NO: 87;
(r) a polypeptide having at least 80% sequence identity to the polypeptide of SEQ ID NO: 90;
(s) a polypeptide having at least 90% sequence identity to the polypeptide of SEQ ID NO: 93;
   or a variant or fragment selected from:
(t) a variant of the polypeptide selected from said first group or said second group wherein the variant has nuclease activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions; and
(u) a fragment of the polypeptide of (a), (b), (c), (d), (e), (f), (g), (h), (i), (j), (k), (I), (m), (n), (o), (p), (q), (r) and (s), wherein said fragment has nuclease activity.

The polypeptide having nuclease activity can be selected from said first group consisting of:
i. a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of any one of SEQ ID NO: 6, 24, 39, 45, 51, 57, 63, 69 and 75; and
ii. a polypeptide having at least 95%, at least 96%, at least 97%, at least 98%, at least 99% or 100% sequence identity to the polypeptide of any one of SEQ ID NO: 21, 27, 42, 54 and 60.

In another embodiment of the invention the polypeptide having nuclease activity is selected from said second group consisting of:
i. a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 78 or 90; and
ii. a polypeptide having at least 95%, at least 96%, at least 97%, at least 98%, at least 99% or 100% sequence identity to the polypeptide of SEQ ID NO: 84 or 93.

In one embodiment of the invention the polypeptide having nuclease activity can be encoded by a polynucleotide having at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to:
i. the mature polypeptide coding sequence of any one of SEQ ID NO: 28, 31, 37, 40, 76, 82, 85, 88, and 91; or
ii. the mature polypeptide cDNA coding sequence of any one of SEQ ID NO: 1, 4, 10, 19, 22, 25, 43, 46, 49, 52, 55, 58, 61, 67 and 73.

In an embodiment of the invention the polypeptide having nuclease activity can be selected from the group consisting of polypeptides:
(a) comprising or consisting of SEQ ID NO: 3 or the mature polypeptide of SEQ ID NO: 2;
(b) comprising or consisting of SEQ ID NO: 6 or the mature polypeptide of SEQ ID NO: 5;
(c) comprising or consisting of SEQ ID NO: 12 or the mature polypeptide of SEQ ID NO: 11;
(d) comprising or consisting of SEQ ID NO: 21 or the mature polypeptide of SEQ ID NO: 20;
(e) comprising or consisting of SEQ ID NO: 24 or the mature polypeptide of SEQ ID NO: 23;
(f) comprising or consisting of SEQ ID NO: 27 or the mature polypeptide of SEQ ID NO: 26;
(g) comprising or consisting of SEQ ID NO: 30 or the mature polypeptide of SEQ ID NO: 29;
(h) comprising or consisting of SEQ ID NO: 33 or the mature polypeptide of SEQ ID NO: 32;
(i) comprising or consisting of SEQ ID NO: 39 or the mature polypeptide of SEQ ID NO: 38;
(j) comprising or consisting of SEQ ID NO: 42 or the mature polypeptide of SEQ ID NO: 41;
(k) comprising or consisting of SEQ ID NO: 45 or the mature polypeptide of SEQ ID NO: 44;
(l) comprising or consisting of SEQ ID NO: 48 or the mature polypeptide of SEQ ID NO: 47;
(m) comprising or consisting of SEQ ID NO: 51 or the mature polypeptide of SEQ ID NO: 50;
(n) comprising or consisting of SEQ ID NO: 54 or the mature polypeptide of SEQ ID NO: 53;
(o) comprising or consisting of SEQ ID NO: 57 or the mature polypeptide of SEQ ID NO: 56;
(p) comprising or consisting of SEQ ID NO: 60 or the mature polypeptide of SEQ ID NO: 59;
(q) comprising or consisting of SEQ ID NO: 63 or the mature polypeptide of SEQ ID NO: 62;
(r) comprising or consisting of SEQ ID NO: 69 or the mature polypeptide of SEQ ID NO: 68;
(s) comprising or consisting of SEQ ID NO: 75 or the mature polypeptide of SEQ ID NO: 74;
(t) comprising or consisting of SEQ ID NO: 78 or the mature polypeptide of SEQ ID NO: 77;
(u) comprising or consisting of SEQ ID NO: 84 or the mature polypeptide of SEQ ID NO: 83;
(v) comprising or consisting of SEQ ID NO: 87 or the mature polypeptide of SEQ ID NO: 86;
(w) comprising or consisting of SEQ ID NO: 90 or the mature polypeptide of SEQ ID NO: 89;
(x) comprising or consisting of SEQ ID NO: 93 or the mature polypeptide of SEQ ID NO: 92;
(y) comprising or consisting of SEQ ID NO: 96 or the mature polypeptide of SEQ ID NO: 95;
(z) comprising or consisting of SEQ ID NO: 99 or the mature polypeptide of SEQ ID NO: 98;
(aa) comprising or consisting of SEQ ID NO: 108 or the mature polypeptide of SEQ ID NO: 107; and
(bb) comprising or consisting of SEQ ID NO: 111 or the mature polypeptide of SEQ ID NO: 110.

In an embodiment, the present invention relates to polypeptides having a sequence identity to the mature polypeptide of SEQ ID NO: 3 of at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 3.

In a particular embodiment the invention relates to polypeptides having a sequence identity to the mature polypeptide of SEQ ID NO: 3 of at least 99%, or 100%, and wherein the polypeptide has at least at least 70% of the nuclease activity of the mature polypeptide of SEQ ID NO: 3.

In a particular embodiment the invention relates to polypeptides having a sequence identity to the mature polypeptide of SEQ ID NO: 3 of at least 99%, or 100%, and wherein the polypeptide has at least at least 75% of the nuclease activity of the mature polypeptide of SEQ ID NO: 3.

In a particular embodiment the invention relates to polypeptides having a sequence identity to the mature polypeptide of SEQ ID NO: 3 of at least 99%, or 100%, and wherein the polypeptide has at least at least 80% of the nuclease activity of the mature polypeptide of SEQ ID NO: 3.

In a particular embodiment the invention relates to polypeptides having a sequence identity to the mature polypeptide of SEQ ID NO: 3 of at least 99%, or 100%, and wherein the polypeptide has at least at least 85% of the nuclease activity of the mature polypeptide of SEQ ID NO: 3.

In a particular embodiment the invention relates to polypeptides having a sequence identity to the mature polypeptide of SEQ ID NO: 3 of at least 99%, or 100%, and wherein the polypeptide has at least at least 90% of the nuclease activity of the mature polypeptide of SEQ ID NO: 3.

In a particular embodiment the invention relates to polypeptides having a sequence identity to the mature polypeptide of SEQ ID NO: 3 of at least 99%, or 100%, and wherein the polypeptide has at least at least 95% of the nuclease activity of the mature polypeptide of SEQ ID NO: 3.

In a particular embodiment the invention relates to polypeptides having a sequence identity to the mature polypeptide of SEQ ID NO: 3 of at least 99%, or 100%, and wherein the polypeptide has at least at least 100% of the nuclease activity of the mature polypeptide of SEQ ID NO: 3.

In an embodiment, the polypeptide has been isolated. A polypeptide of the present invention preferably comprises or consists of the amino acid sequence of SEQ ID NO: 3 or an allelic variant thereof; or is a fragment thereof having nuclease activity. In another aspect, the polypeptide comprises or consists of the mature polypeptide of SEQ ID NO: 3.

In an embodiment, the present invention relates to polypeptides having a sequence identity to the mature polypeptide of SEQ ID NO: 6 of at least 62%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 6.

In a particular embodiment the invention relates to polypeptides having a sequence identity to the mature polypeptide of SEQ ID NO: 6 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 6.

In an embodiment, the polypeptide has been isolated. A polypeptide of the present invention preferably comprises or consists of the amino acid sequence of SEQ ID NO: 6 or an allelic variant thereof; or is a fragment thereof having nuclease activity. In another aspect, the polypeptide comprises or consists of the mature polypeptide of SEQ ID NO: 6.

In an embodiment, the present invention relates to polypeptides having a sequence identity to the mature polypeptide of SEQ ID NO: 12 of at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 12.

In a particular embodiment the invention relates to polypeptides having a sequence identity to the mature polypeptide of SEQ ID NO: 12 at least 97%, at least 98%, at least 99%, or 100%, and wherein the polypeptide has at least at least 70% of the nuclease activity of the mature polypeptide of SEQ ID NO: 12.

In a particular embodiment the invention relates to polypeptides having a sequence identity to the mature polypeptide of SEQ ID NO: 12 of at least 97%, at least 98%, at least 99%, or 100%, and wherein the polypeptide has at least at least 75% of the nuclease activity of the mature polypeptide of SEQ ID NO: 12.

In a particular embodiment the invention relates to polypeptides having a sequence identity to the mature polypeptide of SEQ ID NO: 12 of at least 97%, at least 98%, at least 99%, or 100%, and wherein the polypeptide has at least at least 80% of the nuclease activity of the mature polypeptide of SEQ ID NO: 12.

In a particular embodiment the invention relates to polypeptides having a sequence identity to the mature polypeptide of SEQ ID NO: 12 of at least 97%, at least 98%, at least 99%, or 100%, and wherein the polypeptide has at least at least 85% of the nuclease activity of the mature polypeptide of SEQ ID NO: 12.

In a particular embodiment the invention relates to polypeptides having a sequence identity to the mature polypeptide of SEQ ID NO: 12 of at least 97%, at least 98%, at least 99%, or 100%, and wherein the polypeptide has at least at least 90% of the nuclease activity of the mature polypeptide of SEQ ID NO: 12.

In a particular embodiment the invention relates to polypeptides having a sequence identity to the mature polypeptide of SEQ ID NO: 12 of at least 97%, at least 98%, at least 99%, or 100%, and wherein the polypeptide has at least at least 95% of the nuclease activity of the mature polypeptide of SEQ ID NO: 12.

In a particular embodiment the invention relates to polypeptides having a sequence identity to the mature polypeptide of SEQ ID NO: 12 of at least 97%, at least 98%, at least 99%, or 100%, and wherein the polypeptide has at least at least 100% of the nuclease activity of the mature polypeptide of SEQ ID NO: 12.

In an embodiment, the polypeptide has been isolated. A polypeptide of the present invention preferably comprises or consists of the amino acid sequence of SEQ ID NO: 12 or an allelic variant thereof; or is a fragment thereof having nuclease activity. In another aspect, the polypeptide comprises or consists of the mature polypeptide of SEQ ID NO: 12.

In an embodiment, the present invention relates to polypeptides having a sequence identity to the mature polypeptide of SEQ ID NO: 21 or SEQ ID NO: 27 of at least 86%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 21 or SEQ ID NO: 27.

In a particular embodiment the invention relates to polypeptides having a sequence identity to the mature polypeptide of SEQ ID NO: 21 or SEQ ID NO: 27 of at least 86%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 21 or SEQ ID NO: 27.

In a particular embodiment the invention relates to polypeptides having a sequence identity to the mature polypeptide of SEQ ID NO: 21 or SEQ ID NO: 27 of at least 86%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 21 or SEQ ID NO: 27.

In a particular embodiment the invention relates to polypeptides having a sequence identity to the mature polypeptide of SEQ ID NO: 21 or SEQ ID NO: 27 of at least 86%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 21 or SEQ ID NO: 27.

In a particular embodiment the invention relates to polypeptides having a sequence identity to the mature polypeptide of SEQ ID NO: 21 or SEQ ID NO: 27 of at least 86%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 21 or SEQ ID NO: 27.

In a particular embodiment the invention relates to polypeptides having a sequence identity to the mature polypeptide of SEQ ID NO: 21 or SEQ ID NO: 27 of at least 86%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 21 or SEQ ID NO: 27.

In a particular embodiment the invention relates to polypeptides having a sequence identity to the mature polypeptide of SEQ ID NO: 21 or SEQ ID NO: 27 of at least 86%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 21 or SEQ ID NO: 27.

In a particular embodiment the invention relates to polypeptides having a sequence identity to the mature polypeptide of SEQ ID NO: 21 or SEQ ID NO: 27 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 21 or SEQ ID NO: 27.

In an embodiment, the polypeptide has been isolated. A polypeptide of the present invention preferably comprises or consists of the amino acid sequence of SEQ ID NO: 21 or SEQ ID NO: 27 or an allelic variant thereof; or is a fragment thereof having nuclease activity.

In another aspect, the polypeptide comprises or consists of the mature polypeptide of SEQ ID NO: 21 or SEQ ID NO: 27.

In an embodiment, the present invention relates to polypeptides having a sequence identity to the mature polypeptide of SEQ ID NO: 30 of at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 30.

In a particular embodiment the invention relates to polypeptides having a sequence identity to the mature polypeptide of SEQ ID NO: 30 of at least 98%, at least 99%, or 100%, and wherein the polypeptide has at least at least 70% of the nuclease activity of the mature polypeptide of SEQ ID NO: 30.

In a particular embodiment the invention relates to polypeptides having a sequence identity to the mature polypeptide of SEQ ID NO: 30 of at least 98%, at least 99%, or 100%, and wherein the polypeptide has at least at least 75% of the nuclease activity of the mature polypeptide of SEQ ID NO: 30.

In a particular embodiment the invention relates to polypeptides having a sequence identity to the mature polypeptide of SEQ ID NO: 30 of at least 98%, at least 99%, or 100%, and wherein the polypeptide has at least at least 80% of the nuclease activity of the mature polypeptide of SEQ ID NO: 30.

In a particular embodiment the invention relates to polypeptides having a sequence identity to the mature polypeptide of SEQ ID NO: 30 of at least 98%, at least 99%, or 100%, and wherein the polypeptide has at least at least 85% of the nuclease activity of the mature polypeptide of SEQ ID NO: 30.

In a particular embodiment the invention relates to polypeptides having a sequence identity to the mature polypeptide of SEQ ID NO: 30 of at least 98%, at least 99%, or 100%, and wherein the polypeptide has at least at least 90% of the nuclease activity of the mature polypeptide of SEQ ID NO: 30.

In a particular embodiment the invention relates to polypeptides having a sequence identity to the mature polypeptide of SEQ ID NO: 30 of at least 98%, at least 99%, or 100%, and wherein the polypeptide has at least at least 95% of the nuclease activity of the mature polypeptide of SEQ ID NO: 30.

In a particular embodiment the invention relates to polypeptides having a sequence identity to the mature polypeptide of SEQ ID NO: 30 of at least 98%, at least 99%, or 100%, and wherein the polypeptide has at least at least 100% of the nuclease activity of the mature polypeptide of SEQ ID NO: 30.

In an embodiment, the polypeptide has been isolated. A polypeptide of the present invention preferably comprises or consists of the amino acid sequence of SEQ ID NO: 30 or an allelic variant thereof; or is a fragment thereof having nuclease activity. In another aspect, the polypeptide comprises or consists of the mature polypeptide of SEQ ID NO: 30.

In an embodiment, the present invention relates to polypeptides having a sequence identity of 100% to the mature polypeptide of SEQ ID NO: 33 or SEQ ID NO: 48, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 33 or SEQ ID NO: 48.

In a particular embodiment the invention relates to polypeptides having a sequence identity of 100% to the mature polypeptide of SEQ ID NO: 33 or SEQ ID NO: 48, and wherein the polypeptide has at least at least 70% of the nuclease activity of the mature polypeptide of SEQ ID NO: 33 or SEQ ID NO: 48.

In a particular embodiment the invention relates to polypeptides having a sequence identity of 100% to the mature polypeptide of SEQ ID NO: 33 or SEQ ID NO: 48, and wherein the polypeptide has at least at least 75% of the nuclease activity of the mature polypeptide of SEQ ID NO: 33 or SEQ ID NO: 48.

In a particular embodiment the invention relates to polypeptides having a sequence identity of 100% to the mature polypeptide of SEQ ID NO: 33 or SEQ ID NO: 48, and wherein the polypeptide has at least at least 80% of the nuclease activity of the mature polypeptide of SEQ ID NO: 33 or SEQ ID NO: 48.

In a particular embodiment the invention relates to polypeptides having a sequence identity of 100% to the mature polypeptide of SEQ ID NO: 33 or SEQ ID NO: 48, and wherein the polypeptide has at least at least 85% of the nuclease activity of the mature polypeptide of SEQ ID NO: 33 or SEQ ID NO: 48.

In a particular embodiment the invention relates to polypeptides having a sequence identity of 100% to the mature polypeptide of SEQ ID NO: 33 or SEQ ID NO: 48 and wherein the polypeptide has at least at least 90% of the nuclease activity of the mature polypeptide of SEQ ID NO: 33 or SEQ ID NO: 48.

In a particular embodiment the invention relates to polypeptides having a sequence identity of 100% to the mature polypeptide of SEQ ID NO: 33 or SEQ ID NO: 48, and wherein the polypeptide has at least at least 95% of the nuclease activity of the mature polypeptide of SEQ ID NO: 33 or SEQ ID NO: 48.

In a particular embodiment the invention relates to polypeptides having a sequence identity of 100% to the mature polypeptide of SEQ ID NO: 33 or SEQ ID NO: 48, and wherein the polypeptide has at least at least 100% of the nuclease activity of the mature polypeptide of SEQ ID NO: 33 or SEQ ID NO: 48.

In an embodiment, the polypeptide has been isolated. A polypeptide of the present invention preferably comprises or consists of the amino acid sequence of SEQ ID NO: 33 or SEQ ID NO: 48 or an allelic variant thereof; or is a fragment thereof having nuclease activity.

In another aspect, the polypeptide comprises or consists of the mature polypeptide of SEQ ID NO: 33 or SEQ ID NO: 48 In an embodiment, the present invention relates to polypeptides having a sequence identity to the mature polypeptide of SEQ ID NO: 39 of at least 76%, at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 39.

In a particular embodiment the invention relates to polypeptides having a sequence identity to the mature polypeptide of SEQ ID NO: 39 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 39.

In a particular embodiment the invention relates to polypeptides having a sequence identity to the mature polypeptide of SEQ ID NO: 39 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 39.

In a particular embodiment the invention relates to polypeptides having a sequence identity to the mature polypeptide of SEQ ID NO: 39 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 39.

In a particular embodiment the invention relates to polypeptides having a sequence identity to the mature polypeptide of SEQ ID NO: 39 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 39.

In a particular embodiment the invention relates to polypeptides having a sequence identity to the mature polypeptide of SEQ ID NO: 39 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 39.

In a particular embodiment the invention relates to polypeptides having a sequence identity to the mature polypeptide of SEQ ID NO: 39 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 39.

In a particular embodiment the invention relates to polypeptides having a sequence identity to the mature polypeptide of SEQ ID NO: 39 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 39.

In an embodiment, the polypeptide has been isolated. A polypeptide of the present invention preferably comprises or consists of the amino acid sequence of SEQ ID NO: 39 or an allelic variant thereof; or is a fragment thereof having nuclease activity. In another aspect, the polypeptide comprises or consists of the mature polypeptide of SEQ ID NO: 39.

In an embodiment, the present invention relates to polypeptides having a sequence identity to the mature polypeptide of SEQ ID NO: 42 of at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 42.

In a particular embodiment the invention relates to polypeptides having a sequence identity to the mature polypeptide of SEQ ID NO: 42 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 42.

In a particular embodiment the invention relates to polypeptides having a sequence identity to the mature polypeptide of SEQ ID NO: 42 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 42.

In a particular embodiment the invention relates to polypeptides having a sequence identity to the mature polypeptide of SEQ ID NO: 42 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 42.

In a particular embodiment the invention relates to polypeptides having a sequence identity to the mature polypeptide of SEQ ID NO: 42 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 42.

In a particular embodiment the invention relates to polypeptides having a sequence identity to the mature polypeptide of SEQ ID NO: 42 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 42.

In a particular embodiment the invention relates to polypeptides having a sequence identity to the mature polypeptide of SEQ ID NO: 42 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 42.

In a particular embodiment the invention relates to polypeptides having a sequence identity to the mature polypeptide of SEQ ID NO: 42 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 42.

In an embodiment, the polypeptide has been isolated. A polypeptide of the present invention preferably comprises or consists of the amino acid sequence of SEQ ID NO: 42 or an allelic variant thereof; or is a fragment thereof having nuclease activity. In another aspect, the polypeptide comprises or consists of the mature polypeptide of SEQ ID NO: 42.

In an embodiment, the present invention relates to polypeptides having a sequence identity to the mature polypeptide of SEQ ID NO: 45 or SEQ ID NO: 24 of at least 69%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 45 or SEQ ID NO: 24.

In a particular embodiment the invention relates to polypeptides having a sequence identity to the mature polypeptide of SEQ ID NO: 45 or SEQ ID NO: 24 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 45 or SEQ ID NO: 24.

In a particular embodiment the invention relates to polypeptides having a sequence identity to the mature polypeptide of SEQ ID NO: 45 or SEQ ID NO: 24 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 45 or SEQ ID NO: 24.

In a particular embodiment the invention relates to polypeptides having a sequence identity to the mature polypeptide of SEQ ID NO: 45 or SEQ ID NO: 24 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 45 or SEQ ID NO: 24.

In a particular embodiment the invention relates to polypeptides having a sequence identity to the mature polypeptide of SEQ ID NO: 45 or SEQ ID NO: 24 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 45 or SEQ ID NO: 24.

In a particular embodiment the invention relates to polypeptides having a sequence identity to the mature polypeptide of SEQ ID NO: 45 or SEQ ID NO: 24 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 45 or SEQ ID NO: 24.

In a particular embodiment the invention relates to polypeptides having a sequence identity to the mature polypeptide of SEQ ID NO: 45 or SEQ ID NO: 24 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 45 or SEQ ID NO: 24.

In a particular embodiment the invention relates to polypeptides having a sequence identity to the mature polypeptide of SEQ ID NO: 45 or SEQ ID NO: 24 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 45 or SEQ ID NO: 24.

In an embodiment, the polypeptide has been isolated. A polypeptide of the present invention preferably comprises or consists of the amino acid sequence of SEQ ID NO: 45 or SEQ ID NO: 24 or an allelic variant thereof; or is a fragment thereof having nuclease activity.

In another aspect, the polypeptide comprises or consists of the mature polypeptide of SEQ ID NO: 45 or SEQ ID NO: 24.

In an embodiment, the present invention relates to polypeptides having a sequence identity to the mature polypeptide of SEQ ID NO: 54 of at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 54.

In a particular embodiment the invention relates to polypeptides having a sequence identity to the mature polypeptide of SEQ ID NO: 54 of at least 94%, at least 95%, at least 96%, at least 97%, at least 98%, at least 99%, or 100%, and wherein the polypeptide has at least at least 70% of the nuclease activity of the mature polypeptide of SEQ ID NO: 54.

In a particular embodiment the invention relates to polypeptides having a sequence identity to the mature polypeptide of SEQ ID NO: 54 of at least 94%, at least 95%, at least 96%, at least 97%, at least 98%, at least 99%, or 100%, and wherein the polypeptide has at least at least 75% of the nuclease activity of the mature polypeptide of SEQ ID NO: 54.

In a particular embodiment the invention relates to polypeptides having a sequence identity to the mature polypeptide of SEQ ID NO: 54 of at least 94%, at least 95%, at least 96%, at least 97%, at least 98%, at least 99%, or 100%, and wherein the polypeptide has at least at least 80% of the nuclease activity of the mature polypeptide of SEQ ID NO: 54.

In a particular embodiment the invention relates to polypeptides having a sequence identity to the mature polypeptide of SEQ ID NO: 54 of at least 94%, at least 95%, at least 96%, at least 97%, at least 98%, at least 99%, or 100%, and wherein the polypeptide has at least at least 85% of the nuclease activity of the mature polypeptide of SEQ ID NO: 54.

In a particular embodiment the invention relates to polypeptides having a sequence identity to the mature polypeptide of SEQ ID NO: 54 of at least 94%, at least 95%, at least 96%, at least 97%, at least 98%, at least 99%, or 100%, and wherein the polypeptide has at least at least 90% of the nuclease activity of the mature polypeptide of SEQ ID NO: 54.

In a particular embodiment the invention relates to polypeptides having a sequence identity to the mature polypeptide of SEQ ID NO: 54 of at least 94%, at least 95%, at least 96%, at least 97%, at least 98%, at least 99%, or 100%, and wherein the polypeptide has at least at least 95% of the nuclease activity of the mature polypeptide of SEQ ID NO: 54.

In a particular embodiment the invention relates to polypeptides having a sequence identity to the mature polypeptide of SEQ ID NO: 54 of at least 94%, at least 95%, at least 96%, at least 97%, at least 98%, at least 99%, or 100%, and wherein the polypeptide has at least at least 100% of the nuclease activity of the mature polypeptide of SEQ ID NO: 54.

In an embodiment, the polypeptide has been isolated. A polypeptide of the present invention preferably comprises or consists of the amino acid sequence of SEQ ID NO: 54 or an allelic variant thereof; or is a fragment thereof having nuclease activity. In another aspect, the polypeptide comprises or consists of the mature polypeptide of SEQ ID NO: 54.

In an embodiment, the present invention relates to polypeptides having a sequence identity to the mature polypeptide of SEQ ID NO: 57 of at least 72%, at least 75%, at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 57.

In a particular embodiment the invention relates to polypeptides having a sequence identity to the mature polypeptide of SEQ ID NO: 57 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 57.

In a particular embodiment the invention relates to polypeptides having a sequence identity to the mature polypeptide of SEQ ID NO: 57 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 57.

In a particular embodiment the invention relates to polypeptides having a sequence identity to the mature polypeptide of SEQ ID NO: 57 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 57.

In a particular embodiment the invention relates to polypeptides having a sequence identity to the mature polypeptide of SEQ ID NO: 57 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 57.

In a particular embodiment the invention relates to polypeptides having a sequence identity to the mature polypeptide of SEQ ID NO: 57 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 57.

In a particular embodiment the invention relates to polypeptides having a sequence identity to the mature polypeptide of SEQ ID NO: 57 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 57.

In a particular embodiment the invention relates to polypeptides having a sequence identity to the mature polypeptide of SEQ ID NO: 57 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 57.

In an embodiment, the polypeptide has been isolated. A polypeptide of the present invention preferably comprises or consists of the amino acid sequence of SEQ ID NO: 57 or an allelic variant thereof; or is a fragment thereof having nuclease activity. In another aspect, the polypeptide comprises or consists of the mature polypeptide of SEQ ID NO: 57.

In an embodiment, the present invention relates to polypeptides having a sequence identity to the mature polypeptide of SEQ ID NO: 60 of at least 82%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 60.

In a particular embodiment the invention relates to polypeptides having a sequence identity to the mature polypeptide of SEQ ID NO: 60 of at least 82%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 60.

In a particular embodiment the invention relates to polypeptides having a sequence identity to the mature polypeptide of SEQ ID NO: 60 of at least 82%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 60.

In a particular embodiment the invention relates to polypeptides having a sequence identity to the mature polypeptide of SEQ ID NO: 60 of at least 82%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 60.

In a particular embodiment the invention relates to polypeptides having a sequence identity to the mature polypeptide of SEQ ID NO: 60 of at least 82%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 60.

In a particular embodiment the invention relates to polypeptides having a sequence identity to the mature polypeptide of SEQ ID NO: 60 of at least 82%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 60.

In a particular embodiment the invention relates to polypeptides having a sequence identity to the mature polypeptide of SEQ ID NO: 60 of at least 82%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 60.

In a particular embodiment the invention relates to polypeptides having a sequence identity to the mature polypeptide of SEQ ID NO: 60 of at least 82%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 60.

In an embodiment, the polypeptide has been isolated. A polypeptide of the present invention preferably comprises or consists of the amino acid sequence of SEQ ID NO: 60 or an allelic variant thereof; or is a fragment thereof having nuclease activity. In another aspect, the polypeptide comprises or consists of the mature polypeptide of SEQ ID NO: 60.

In an embodiment, the present invention relates to polypeptides having a sequence identity to the mature polypeptide of SEQ ID NO: 63 of at least 75%, at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 63.

In a particular embodiment the invention relates to polypeptides having a sequence identity to the mature polypeptide of SEQ ID NO: 63 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 63.

In a particular embodiment the invention relates to polypeptides having a sequence identity to the mature polypeptide of SEQ ID NO: 63 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 63.

In a particular embodiment the invention relates to polypeptides having a sequence identity to the mature polypeptide of SEQ ID NO: 63 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 63.

In a particular embodiment the invention relates to polypeptides having a sequence identity to the mature polypeptide of SEQ ID NO: 63 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 63.

In a particular embodiment the invention relates to polypeptides having a sequence identity to the mature polypeptide of SEQ ID NO: 63 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 63.

In a particular embodiment the invention relates to polypeptides having a sequence identity to the mature polypeptide of SEQ ID NO: 63 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 63.

In a particular embodiment the invention relates to polypeptides having a sequence identity to the mature polypeptide of SEQ ID NO: 63 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 63.

In an embodiment, the polypeptide has been isolated. A polypeptide of the present invention preferably comprises or consists of the amino acid sequence of SEQ ID NO: 63 or an allelic variant thereof; or is a fragment thereof having nuclease activity. In another aspect, the polypeptide comprises or consists of the mature polypeptide of SEQ ID NO: 63.

In an embodiment, the present invention relates to polypeptides having a sequence identity to the mature polypeptide of SEQ ID NO: 69 or SEQ ID NO: 75 of at least 80%, at least 85%, at least 90%, at least 91%, at least 92%, at least 93%, at least 94%, at least 95%, at least 96%, at least 97%, at least 98%, at least 99%, or 100%, which have nuclease activity.

In one aspect, the polypeptides differ by up to 10 amino acids, e.g., 1, 2, 3, 4, 5, 6, 7, 8, 9, or 10, from the mature polypeptide of SEQ ID NO: 69 or SEQ ID NO: 75.

In a particular embodiment the invention relates to polypeptides having a sequence identity to the mature polypeptide of SEQ ID NO: 69 or SEQ ID NO: 75 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 69 or SEQ ID NO: 75.

In a particular embodiment the invention relates to polypeptides having a sequence identity to the mature polypeptide of SEQ ID NO: 69 or SEQ ID NO: 75 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 69 or SEQ ID NO: 75.

In a particular embodiment the invention relates to polypeptides having a sequence identity to the mature polypeptide of SEQ ID NO: 69 or SEQ ID NO: 75 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 69 or SEQ ID NO: 75.

In a particular embodiment the invention relates to polypeptides having a sequence identity to the mature polypeptide of SEQ ID NO: 69 or SEQ ID NO: 75 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 69 or SEQ ID NO: 75.

In a particular embodiment the invention relates to polypeptides having a sequence identity to the mature polypeptide of SEQ ID NO: 69 or SEQ ID NO: 75 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 69 or SEQ ID NO: 75.

In a particular embodiment the invention relates to polypeptides having a sequence identity to the mature polypeptide of SEQ ID NO: 69 or SEQ ID NO: 75 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 69 or SEQ ID NO: 75.

In a particular embodiment the invention relates to polypeptides having a sequence identity to the mature polypeptide of SEQ ID NO: 69 or SEQ ID NO: 75 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 69 or SEQ ID NO: 75.

In an embodiment, the polypeptide has been isolated. A polypeptide of the present invention preferably comprises or consists of the amino acid sequence of SEQ ID NO: 69 or SEQ ID NO: 75 or an allelic variant thereof; or is a fragment thereof having nuclease activity.

In another aspect, the polypeptide comprises or consists of the mature polypeptide of SEQ ID NO: 69 or SEQ ID NO: 75.

In an embodiment, the present invention relates to polypeptides having a sequence identity to the mature polypeptide of SEQ ID NO: 78 of at least 77%, at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 78.

In a particular embodiment the invention relates to polypeptides having a sequence identity to the mature polypeptide of SEQ ID NO: 78 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 78.

In a particular embodiment the invention relates to polypeptides having a sequence identity to the mature polypeptide of SEQ ID NO: 78 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 78.

In a particular embodiment the invention relates to polypeptides having a sequence identity to the mature polypeptide of SEQ ID NO: 78 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 78.

In a particular embodiment the invention relates to polypeptides having a sequence identity to the mature polypeptide of SEQ ID NO: 78 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 78.

In a particular embodiment the invention relates to polypeptides having a sequence identity to the mature polypeptide of SEQ ID NO: 78 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 78.

In a particular embodiment the invention relates to polypeptides having a sequence identity to the mature polypeptide of SEQ ID NO: 78 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 78.

In a particular embodiment the invention relates to polypeptides having a sequence identity to the mature polypeptide of SEQ ID NO: 78 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 78.

In an embodiment, the polypeptide has been isolated. A polypeptide of the present invention preferably comprises or consists of the amino acid sequence of SEQ ID NO: 78 or an allelic variant thereof; or is a fragment thereof having nuclease activity. In another aspect, the polypeptide comprises or consists of the mature polypeptide of SEQ ID NO: 78.

In an embodiment, the present invention relates to polypeptides having a sequence identity to the mature polypeptide of SEQ ID NO: 84 of at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 84.

In a particular embodiment the invention relates to polypeptides having a sequence identity to the mature polypeptide of SEQ ID NO: 84 at least 94%, at least 95%, at least 96%, at least 97%, at least 98%, at least 99%, or 100%, and wherein the polypeptide has at least at least 70% of the nuclease activity of the mature polypeptide of SEQ ID NO: 84.

In a particular embodiment the invention relates to polypeptides having a sequence identity to the mature polypeptide of SEQ ID NO: 84 of at least 94%, at least 95%, at least 96%, at least 97%, at least 98%, at least 99%, or 100%, and wherein the polypeptide has at least at least 75% of the nuclease activity of the mature polypeptide of SEQ ID NO: 84.

In a particular embodiment the invention relates to polypeptides having a sequence identity to the mature polypeptide of SEQ ID NO: 84 of at least 94%, at least 95%, at least 96%, at least 97%, at least 98%, at least 99%, or 100%, and wherein the polypeptide has at least at least 80% of the nuclease activity of the mature polypeptide of SEQ ID NO: 84.

In a particular embodiment the invention relates to polypeptides having a sequence identity to the mature polypeptide of SEQ ID NO: 84 of at least 94%, at least 95%, at least 96%, at least 97%, at least 98%, at least 99%, or 100%, and wherein the polypeptide has at least at least 85% of the nuclease activity of the mature polypeptide of SEQ ID NO: 84.

In a particular embodiment the invention relates to polypeptides having a sequence identity to the mature polypeptide of SEQ ID NO: 84 of at least 94%, at least 95%, at least 96%, at least 97%, at least 98%, at least 99%, or 100%, and wherein the polypeptide has at least at least 90% of the nuclease activity of the mature polypeptide of SEQ ID NO: 84.

In a particular embodiment the invention relates to polypeptides having a sequence identity to the mature polypeptide of SEQ ID NO: 84 of at least 94%, at least 95%, at least 96%, at least 97%, at least 98%, at least 99%, or 100%, and wherein the polypeptide has at least at least 95% of the nuclease activity of the mature polypeptide of SEQ ID NO: 84.

In a particular embodiment the invention relates to polypeptides having a sequence identity to the mature polypeptide of SEQ ID NO: 84 of at least 94%, at least 95%, at least 96%, at least 97%, at least 98%, at least 99%, or 100%, and wherein the polypeptide has at least at least 100% of the nuclease activity of the mature polypeptide of SEQ ID NO: 84.

In an embodiment, the polypeptide has been isolated. A polypeptide of the present invention preferably comprises or consists of the amino acid sequence of SEQ ID NO: 84 or an allelic variant thereof; or is a fragment thereof having nuclease activity. In another aspect, the polypeptide comprises or consists of the mature polypeptide of SEQ ID NO: 84.

In an embodiment, the present invention relates to polypeptides having a sequence identity to the mature polypeptide of SEQ ID NO: 87 of at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 87.

In a particular embodiment the invention relates to polypeptides having a sequence identity to the mature polypeptide of SEQ ID NO: 87 of at least 99%, or 100%, and wherein the polypeptide has at least at least 70% of the nuclease activity of the mature polypeptide of SEQ ID NO: 87.

In a particular embodiment the invention relates to polypeptides having a sequence identity to the mature polypeptide of SEQ ID NO: 87 of at least 99%, or 100%, and wherein the polypeptide has at least at least 75% of the nuclease activity of the mature polypeptide of SEQ ID NO: 87.

In a particular embodiment the invention relates to polypeptides having a sequence identity to the mature polypeptide of SEQ ID NO: 87 of at least 99%, or 100%, and wherein the polypeptide has at least at least 80% of the nuclease activity of the mature polypeptide of SEQ ID NO: 87.

In a particular embodiment the invention relates to polypeptides having a sequence identity to the mature polypeptide of SEQ ID NO: 87 of at least 99%, or 100%, and wherein the polypeptide has at least at least 85% of the nuclease activity of the mature polypeptide of SEQ ID NO: 87.

In a particular embodiment the invention relates to polypeptides having a sequence identity to the mature polypeptide of SEQ ID NO: 87 of at least 99%, or 100%, and wherein the polypeptide has at least at least 90% of the nuclease activity of the mature polypeptide of SEQ ID NO: 87.

In a particular embodiment the invention relates to polypeptides having a sequence identity to the mature polypeptide of SEQ ID NO: 87 of at least 99%, or 100%, and wherein the polypeptide has at least at least 95% of the nuclease activity of the mature polypeptide of SEQ ID NO: 87.

In a particular embodiment the invention relates to polypeptides having a sequence identity to the mature polypeptide of SEQ ID NO: 87 of at least 99%, or 100%, and wherein the polypeptide has at least at least 100% of the nuclease activity of the mature polypeptide of SEQ ID NO: 87.

In an embodiment, the polypeptide has been isolated. A polypeptide of the present invention preferably comprises or consists of the amino acid sequence of SEQ ID NO: 87 or an allelic variant thereof; or is a fragment thereof having nuclease activity. In another aspect, the polypeptide comprises or consists of the mature polypeptide of SEQ ID NO: 87.

In an embodiment, the present invention relates to polypeptides having a sequence identity to the mature polypeptide of SEQ ID NO: 90 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 90.

In a particular embodiment the invention relates to polypeptides having a sequence identity to the mature polypeptide of SEQ ID NO: 90 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 90.

In a particular embodiment the invention relates to polypeptides having a sequence identity to the mature polypeptide of SEQ ID NO: 90 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 90.

In a particular embodiment the invention relates to polypeptides having a sequence identity to the mature polypeptide of SEQ ID NO: 90 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 90.

In a particular embodiment the invention relates to polypeptides having a sequence identity to the mature polypeptide of SEQ ID NO: 90 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 90.

In a particular embodiment the invention relates to polypeptides having a sequence identity to the mature polypeptide of SEQ ID NO: 90 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 90.

In a particular embodiment the invention relates to polypeptides having a sequence identity to the mature polypeptide of SEQ ID NO: 90 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 90.

In a particular embodiment the invention relates to polypeptides having a sequence identity to the mature polypeptide of SEQ ID NO: 90 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 90.

In an embodiment, the polypeptide has been isolated. A polypeptide of the present invention preferably comprises or consists of the amino acid sequence of SEQ ID NO: 90 or an allelic variant thereof; or is a fragment thereof having nuclease activity. In another aspect, the polypeptide comprises or consists of the mature polypeptide of SEQ ID NO: 90.

In an embodiment, the present invention relates to polypeptides having a sequence identity to the mature polypeptide of SEQ ID NO: 93 of at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 93.

In a particular embodiment the invention relates to polypeptides having a sequence identity to the mature polypeptide of SEQ ID NO: 93 of at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 93.

In a particular embodiment the invention relates to polypeptides having a sequence identity to the mature polypeptide of SEQ ID NO: 93 of at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 93.

In a particular embodiment the invention relates to polypeptides having a sequence identity to the mature polypeptide of SEQ ID NO: 93 of at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 93.

In a particular embodiment the invention relates to polypeptides having a sequence identity to the mature polypeptide of SEQ ID NO: 93 of at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 93.

In a particular embodiment the invention relates to polypeptides having a sequence identity to the mature polypeptide of SEQ ID NO: 93 of at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 93.

In a particular embodiment the invention relates to polypeptides having a sequence identity to the mature polypeptide of SEQ ID NO: 93 of at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 93.

In a particular embodiment the invention relates to polypeptides having a sequence identity to the mature polypeptide of SEQ ID NO: 93 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 93.

In an embodiment, the polypeptide has been isolated. A polypeptide of the present invention preferably comprises or consists of the amino acid sequence of SEQ ID NO: 93 or an allelic variant thereof; or is a fragment thereof having nuclease activity. In another aspect, the polypeptide comprises or consists of the mature polypeptide of SEQ ID NO: 93.

The polynucleotide of SEQ ID NO: 1, 4, 7, 10, 13, 16, 19, 22, 25, 28, 31, 34, 37, 40, 43, 46, 49, 52, 55, 76, 79, 82, 85, 88, 91, 94, or 97 or a subsequence thereof, as well as the polypeptide of SEQ ID NO: 2, 5, 8, 11, 14, 17, 20, 23, 26, 29, 32, 35, 38, 41, 44, 47, 50, 53, 56, 77, 80, 83, 86, 89, 92, 95, or 96 or a fragment thereof may be used to design nucleic acid probes to identify and clone DNA encoding polypeptides having nuclease activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having nuclease activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1 or a subsequence thereof, the carrier material is used in a Southern blot.

In another embodiment, the present invention relates to a polypeptide having nuclease activity encoded by a polynucleotide having a sequence identity to the mature polypeptide coding sequence of SEQ ID NO: 1, SEQ ID NO: 4, SEQ ID NO: 7, SEQ ID NO: 10, SEQ ID NO: 13, or SEQ ID NO: 16 SEQ ID NO: 19, SEQ ID NO: 22, SEQ ID NO: 25, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73 or the cDNA sequence thereof of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to a polypeptide having nuclease activity encoded by a polynucleotide having a sequence identity to the mature polypeptide coding sequence of SEQ ID NO: 28, SEQ ID NO: 31, SEQ ID NO: 34, SEQ ID NO: 37, SEQ ID NO: 40, SEQ ID NO: 43, SEQ ID NO: 76, SEQ ID NO: 79, SEQ ID NO: 82, SEQ ID NO: 85, SEQ ID NO: 88, SEQ ID NO: 91, SEQ ID NO: 94 or SEQ ID NO: 97 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%. In a further embodiment, the polypeptide has been isolated.

In another embodiment, the present invention relates to variants of the mature polypeptide of SEQ ID NO: 1, SEQ ID NO: 4, SEQ ID NO: 7, SEQ ID NO: 10, SEQ ID NO: 13, SEQ ID NO: 16, SEQ ID NO: 19, SEQ ID NO: 22, SEQ ID NO: 25, SEQ ID NO: 28, SEQ ID NO: 31, SEQ ID NO: 34, SEQ ID NO: 37, SEQ ID NO: 40, SEQ ID NO: 43, SEQ ID NO: 46, SEQ ID NO: 49, SEQ ID NO: 52, SEQ ID NO: 55, SEQ ID NO: 58, SEQ ID NO: 61, SEQ ID NO: 64, SEQ ID NO: 67, SEQ ID NO: 70, SEQ ID NO: 73, SEQ ID NO: 76, SEQ ID NO: 79, SEQ ID NO: 82, SEQ ID NO: 85, SEQ ID NO: 88, SEQ ID NO: 91, SEQ ID NO: 94 or SEQ ID NO: 97 comprising a substitution, deletion, and/or insertion at one or more positions. In an embodiment, the number of amino acid substitutions, deletions and/or insertions introduced into the mature polypeptide of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 108 or SEQ ID NO: 111 is up to 10, e.g., 1, 2, 3, 4, 5, 6, 7, 8, 9, or 10. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/11e, Leu/Val, Ala/Glu, and Asp/Gly.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant molecules are tested for nuclease activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al., 1986, Gene 46:145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al., 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Nuclease Activity

A polypeptide having nuclease activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

In one aspect the polypeptide having nuclease activity may be obtained from Bacillus, Penicillium, Streptomyces, Acremonium, Daldinia, Actinomucor, Talaromyces, Lysobacter, Pseudoalteromonas, Vibrio, Janthinobacterium, Massilia, Aspergillus, Pyrenochaetopsis Lachnellula, Trichoderma, Cordyceps, Acremonium, Microdochium, Morchella, Stenocarpella, Cordyceps, Phialophora, or an Adophora cell.

In one aspect, the polypeptide having nuclease activity may be obtained from Bacillus, e.g., from Bacillus sp-62738, Bacillus deramificans or Bacillus thuringiensis.

In one aspect, the polypeptide having nuclease activity may be obtained from Penicillium, e.g., from Penicillium virgatum, Penicillium atramentosum, Penicillium emersonii or Penicillium cremeogriseum.

In one aspect, the polypeptide having nuclease activity may be obtained from Streptomyces, e.g., Streptomyces cirratus.

In one aspect, the polypeptide having nuclease activity may be obtained from Acremonium, e.g., Acremonium sp. XZ1968 or Acremonium alcalophilum.

In one aspect, the polypeptide having nuclease activity may be obtained from Daldinia, e.g., Daldinia fissa.

In one aspect, the polypeptide having nuclease activity may be obtained from Actinomucor, e.g., Actinomucor elegans.

In one aspect, the polypeptide having nuclease activity may be obtained from Talaromyces, e.g., Talaromyces leycettanus.

In one aspect, the polypeptide having nuclease activity may be obtained from Lysobacter, e.g., Lysobacter enzymogenes.

In one aspect, the polypeptide having nuclease activity may be obtained from Pseudoalteromonas, e.g., Pseudoalteromonas nigrifaciens.

In one aspect, the polypeptide having nuclease activity may be obtained from Vibrio, e.g., Vibrio sp-63684.

In one aspect, the polypeptide having nuclease activity may be obtained from Janthinobacterium, e.g., Janthinobacterium agaricidamnosum.

In one aspect, the polypeptide having nuclease activity may be obtained from Massilia, e.g., Massilia aerilata.

In one aspect, the polypeptide having nuclease activity may be obtained from Aspergillus, e.g., Aspergillus oryzae.

In one aspect, the polypeptide having nuclease activity may be obtained from Ostropa, e.g., Ostropa barbara.

In one aspect, the polypeptide having nuclease activity may be obtained from Lachnellula, e.g., Pyrenochaetopsis sp.

In one aspect, the polypeptide having nuclease activity may be obtained from Pyrenochaetopsis, e.g., Lachnellula sp.

In one aspect, the polypeptide having nuclease activity may be obtained from Trichoderma, e.g., Trichoderma reesei or Trichoderma hamatum.

In one aspect, the polypeptide having nuclease activity may be obtained from Cordyceps, e.g., Cordyceps cardinalis.

In one aspect, the polypeptide having nuclease activity may be obtained from Microdochium, e.g., Microdochium phragmitis.

In one aspect, the polypeptide having nuclease activity may be obtained from Morchella, e.g., Morchella costata.

In one aspect, the polypeptide having nuclease activity may be obtained from Stenocarpella, e.g., Stenocarpella maydis.

In one aspect, the polypeptide having nuclease activity may be obtained from Phialophora, e.g., Phialophora geniculate.

In one aspect, the polypeptide having nuclease activity may be obtained from Cadophora, e.g., Cadophora fastigiata.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms isolated from nature (e.g., soil, composts, water, etc.) or DNA samples obtained directly from natural materials (e.g., soil, composts, water, etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Polynucleotides

The present invention also relates to polynucleotides encoding a polypeptide of the present invention, as described herein. In an embodiment, the polynucleotide encoding the polypeptide of the present invention has been isolated.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Bacillus, Penicillium, Streptomyces, Acremonium, Daldinia, Actinomucor, Talaromyces, Lysobacter, Pseudoalteromonas, Vibrio, Janthinobacterium, Massilia, Aspergillus, Pyrenochaetopsis Lachnellula, Trichoderma, Cordyceps, Acremonium, Microdochium, Morchella, Stenocarpella, Cordyceps, Phialophora, Adophora or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

The polynucleotide may be manipulated in a variety of ways to provide for expression of the polypeptide. Manipulation of the polynucleotide prior to its insertion into a vector may be desirable or necessary depending on the expression vector. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including variant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and variant, truncated, and hybrid promoters thereof. Other promoters are described in U.S. Pat. No. 6,011,147.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rrnB).

Preferred terminators for filamentous fungal host cells are obtained from the genes for Aspergillus nidulans acetamidase, Aspergillus nidulans anthranilate synthase, Aspergillus niger glucoamylase, Aspergillus niger alpha-glucosidase, Aspergillus oryzae TAKA amylase, Fusarium oxysporum trypsin-like protease, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase V, Trichoderma reesei xylanase I, Trichoderma reesei xylanase II, Trichoderma reesei xylanase III, Trichoderma reesei beta-xylosidase, and Trichoderma reesei translation elongation factor.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory sequences are those that cause expression of the gene to be turned on or off in response to a chemical or physical stimulus, including the presence of a regulatory compound. Regulatory sequences in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter, Trichoderma reesei cellobiohydrolase I promoter, and Trichoderma reesei cellobiohydrolase II promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked to the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated.

Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosylaminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene. Preferred for use in a Trichoderma cell are adeA, adeB, amdS, hph, and pyrG genes.

The selectable marker may be a dual selectable marker system as described in WO 2010/039889. In one aspect, the dual selectable marker is an hph-tk dual selectable marker system.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMf31 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus altitudinis, Bacillus amyloliquefaciens, B. amyloliquefaciens subsp. plantarum, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus methylotrophicus, Bacillus pumilus, Bacillus safensis, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thorne, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al., 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and optionally, (b) recovering the polypeptide. In one aspect, the cell is a Bacillus, Penicillium, Streptomyces, Acremonium, Daldinia, Actinomucor, Talaromyces, Lysobacter, Pseudoalteromonas, Vibrio, Janthinobacterium, Massilia, Aspergillus, Pyrenochaetopsis Lachnellula, Trichoderma, Cordyceps, Acremonium, Microdochium, Morchella, Stenocarpella, Cordyceps, Phialophora, or an Adophora cell.

In another aspect, the cell is a Bacillus cell. In another aspect, the cell is Bacillus sp-62738, Bacillus deramificans or Bacillus thuringiensis.

In another aspect, the cell is a Penicillium cell. In another aspect, the cell is Penicillium virgatum, Penicillium atramentosum, Penicillium emersonii or Penicillium cremeogriseum.

In another aspect, the cell is a Streptomyces cell. In another aspect, the cell is Streptomyces cirratus.

In another aspect, the cell is a Acremonium cell. In another aspect, the cell is Acremonium sp. XZ1968 or Acremonium alcalophilum.

In another aspect, the cell is a Daldinia cell. In another aspect, the cell is Daldinia fissa.

In another aspect, the cell is a Actinomucor cell. In another aspect, the cell is Actinomucor elegans.

In another aspect, the cell is a Talaromyces cell. In another aspect, the cell is Talaromyces leycettanus.

In another aspect, the cell is a Lysobacter cell. In another aspect, the cell is Lysobacter enzymogenes.

In another aspect, the cell is a Pseudoalteromonas cell. In another aspect, the cell is Pseudoalteromonas nigrifaciens.

In another aspect, the cell is a Vibrio cell. In another aspect, the cell is Vibrio sp-63684.

In another aspect, the cell is a Janthinobacterium cell. In another aspect, the cell is Janthinobacterium agaricidamnosum.

In another aspect, the cell is a Massilia cell. In another aspect, the cell is Massilia aerilata.

In another aspect, the cell is a Aspergillus cell. In another aspect, the cell is Aspergillus oryzae.

In another aspect, the cell is a Ostropa cell. In another aspect, the cell is Ostropa Barbara.

In another aspect, the cell is a Lachnellula cell. In another aspect, the cell is Pyrenochaetopsis sp.

In another aspect, the cell is a Pyrenochaetopsis cell. In another aspect, the cell is Lachnellula sp.

In another aspect, the cell is a Trichoderma cell. In another aspect, the cell is Trichoderma reesei or Trichoderma hamatum.

In another aspect, the cell is a Cordyceps cell. In another aspect, the cell is Cordyceps cardinalis.

In another aspect, the cell is a Microdochium cell. In another aspect, the cell is Microdochium phragmitis.

In another aspect, the cell is a Morchella cell. In another aspect, the cell is Morchella costata.

In another aspect, the cell is a Stenocarpella cell. In another aspect, the cell is Stenocarpella maydis.

In another aspect, the cell is a Phialophora cell. In another aspect, the cell is Phialophora geniculate.

In another aspect, the cell is a Cadophora cell. In another aspect, the cell is Cadophora fastigiata.

The present invention also relates to methods of producing a polypeptide of the present invention, comprising (a) cultivating a recombinant host cell of the present invention under conditions conducive for production of the polypeptide; and optionally, (b) recovering the polypeptide.

The host cells are cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermentors in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides having nuclease activity. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Fermentation Broth Formulations or Cell Compositions

The present invention also relates to a fermentation broth formulation or a cell composition comprising a polypeptide of the present invention. The fermentation broth product further comprises additional ingredients used in the fermentation process, such as, for example, cells (including, the host cells containing the gene encoding the polypeptide of the present invention which are used to produce the polypeptide of interest), cell debris, biomass, fermentation media and/or fermentation products. In some embodiments, the composition is a cell-killed whole broth containing organic acid(s), killed cells and/or cell debris, and culture medium.

The term “fermentation broth” as used herein refers to a preparation produced by cellular fermentation that undergoes no or minimal recovery and/or purification. For example, fermentation broths are produced when microbial cultures are grown to saturation, incubated under carbon-limiting conditions to allow protein synthesis (e.g., expression of enzymes by host cells) and secretion into cell culture medium. The fermentation broth can contain unfractionated or fractionated contents of the fermentation materials derived at the end of the fermentation. Typically, the fermentation broth is unfractionated and comprises the spent culture medium and cell debris present after the microbial cells (e.g., filamentous fungal cells) are removed, e.g., by centrifugation. In some embodiments, the fermentation broth contains spent cell culture medium, extracellular enzymes, and viable and/or nonviable microbial cells.

In an embodiment, the fermentation broth formulation and cell compositions comprise a first organic acid component comprising at least one 1-5 carbon organic acid and/or a salt thereof and a second organic acid component comprising at least one 6 or more carbon organic acid and/or a salt thereof. In a specific embodiment, the first organic acid component is acetic acid, formic acid, propionic acid, a salt thereof, or a mixture of two or more of the foregoing and the second organic acid component is benzoic acid, cyclohexanecarboxylic acid, 4-methylvaleric acid, phenylacetic acid, a salt thereof, or a mixture of two or more of the foregoing.

In one aspect, the composition contains an organic acid(s), and optionally further contains killed cells and/or cell debris. In one embodiment, the killed cells and/or cell debris are removed from a cell-killed whole broth to provide a composition that is free of these components.

The fermentation broth formulations or cell compositions may further comprise a preservative and/or anti-microbial (e.g., bacteriostatic) agent, including, but not limited to, sorbitol, sodium chloride, potassium sorbate, and others known in the art.

The cell-killed whole broth or composition may contain the unfractionated contents of the fermentation materials derived at the end of the fermentation. Typically, the cell-killed whole broth or composition contains the spent culture medium and cell debris present after the microbial cells (e.g., filamentous fungal cells) are grown to saturation, incubated under carbon-limiting conditions to allow protein synthesis. In some embodiments, the cell-killed whole broth or composition contains the spent cell culture medium, extracellular enzymes, and killed filamentous fungal cells. In some embodiments, the microbial cells present in the cell-killed whole broth or composition can be permeabilized and/or lysed using methods known in the art.

A whole broth or cell composition as described herein is typically a liquid, but may contain insoluble components, such as killed cells, cell debris, culture media components, and/or insoluble enzyme(s). In some embodiments, insoluble components may be removed to provide a clarified liquid composition.

The whole broth formulations and cell compositions of the present invention may be produced by a method described in WO 90/15861 or WO 2010/096673.

### Enzyme Compositions

The invention further comprises a composition comprising a polypeptide of the invention. In one embodiment the composition comprises a polypeptide having nuclease activity, selected from a first group consisting of:
(a) a polypeptide having at least 99% sequence identity to the polypeptide of SEQ ID NO: 3;
(b) a polypeptide having at least 62% sequence identity to the polypeptide of SEQ ID NO: 6;
(c) a polypeptide having at least 97% sequence identity to the polypeptide of SEQ ID NO: 12; and
(d) a polypeptide having at least 86% sequence identity to the polypeptide of SEQ ID NO: 21 or 27;
(e) a polypeptide having at least 98% sequence identity to the polypeptide of SEQ ID NO: 30;
(f) a polypeptide having 100% sequence identity to the polypeptide of SEQ ID NO: 33 or 48;
(g) a polypeptide having at least 76% sequence identity to the polypeptide of SEQ ID NO: 39 or 51;
(h) a polypeptide having at least 95% sequence identity to the polypeptide of SEQ ID NO: 42;
(i) a polypeptide having at least 79% sequence identity to the polypeptide of SEQ ID NO: 45 or 24;
(j) a polypeptide having at least 94% sequence identity to the polypeptide of SEQ ID NO: 54;
(k) a polypeptide having at least 72% sequence identity to the polypeptide of SEQ ID NO: 57;
(l) a polypeptide having at least 82% sequence identity to the polypeptide of SEQ ID NO: 60;
(m) a polypeptide having at least 75% sequence identity to the polypeptide of SEQ ID NO: 63;
(n) a polypeptide having at least 80% sequence identity to the polypeptide of SEQ ID NO: 69 or 75;
   or selected from a second group consisting of:
(o) a polypeptide having at least 77% sequence identity to the polypeptide of SEQ ID NO: 78;
(p) a polypeptide having at least 94% sequence identity to the polypeptide of SEQ ID NO: 84;
(q) a polypeptide having at least 99% sequence identity to the polypeptide of SEQ ID NO: 87;
(r) a polypeptide having at least 80% sequence identity to the polypeptide of SEQ ID NO: 90; and
(s) a polypeptide having at least 90% sequence identity to the polypeptide of SEQ ID NO: 93;
   or a variant or fragment selected from:
(t) a variant of the polypeptide selected from said first group or said second group wherein the variant has nuclease activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions;
(u) a fragment of the polypeptide of (a), (b), (c), (d), (e), (f), (g), (h), (i), (j), (k), (l), (m), (n), (o), (p), (q), (r) and (s), wherein said fragment has nuclease activity.

In one embodiment of the invention, the composition comprises a polypeptide selected from the group consisting of:
i. a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of any one of SEQ ID NO: 6, 24, 39, 45, 51, 57, 63, 69 and 75; and
ii. a polypeptide having at least 95%, at least 96%, at least 97%, at least 98%, at least 99% or 100% sequence identity to the polypeptide of any one of SEQ ID NO: 21, 27, 42, 54 and 60.

In one embodiment of the invention, the composition comprises a polypeptide selected from the group consisting of:
i. a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 78 or 90; and
ii. a polypeptide having at least 95%, at least 96%, at least 97%, at least 98%, at least 99% or 100% sequence identity to the polypeptide of SEQ ID NO: 84 or 93.

In one embodiment of the invention, the composition comprises a polypeptide which is encoded by a polynucleotide having at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to:
i. the mature polypeptide coding sequence of any one of SEQ ID NO: 28, 31, 37, 40, 76, 82, 85, 88, and 91; or
ii. the mature polypeptide cDNA coding sequence of any one of SEQ ID NO: 1, 4, 10, 19, 22, 25, 43, 46, 49, 52, 55, 58, 61, 67 and 73.

In one embodiment of the invention, the composition comprises a polypeptide selected from the group consisting of polypeptides:
(a) comprising or consisting of SEQ ID NO: 3 or the mature polypeptide of SEQ ID NO: 2;
(b) comprising or consisting of SEQ ID NO: 6 or the mature polypeptide of SEQ ID NO: 5;
(c) comprising or consisting of SEQ ID NO: 12 or the mature polypeptide of SEQ ID NO: 11;
(d) comprising or consisting of SEQ ID NO: 21 or the mature polypeptide of SEQ ID NO: 20;
(e) comprising or consisting of SEQ ID NO: 24 or the mature polypeptide of SEQ ID NO: 23;
(f) comprising or consisting of SEQ ID NO: 27 or the mature polypeptide of SEQ ID NO: 26;
(g) comprising or consisting of SEQ ID NO: 30 or the mature polypeptide of SEQ ID NO: 29;
(h) comprising or consisting of SEQ ID NO: 33 or the mature polypeptide of SEQ ID NO: 32;
(i) comprising or consisting of SEQ ID NO: 39 or the mature polypeptide of SEQ ID NO: 38;
(j) comprising or consisting of SEQ ID NO: 42 or the mature polypeptide of SEQ ID NO: 41;
(k) comprising or consisting of SEQ ID NO: 45 or the mature polypeptide of SEQ ID NO: 44;
(l) comprising or consisting of SEQ ID NO: 48 or the mature polypeptide of SEQ ID NO: 47;
(m) comprising or consisting of SEQ ID NO: 51 or the mature polypeptide of SEQ ID NO: 50;
(n) comprising or consisting of SEQ ID NO: 54 or the mature polypeptide of SEQ ID NO: 53;
(o) comprising or consisting of SEQ ID NO: 57 or the mature polypeptide of SEQ ID NO: 56;
(p) comprising or consisting of SEQ ID NO: 60 or the mature polypeptide of SEQ ID NO: 59;
(q) comprising or consisting of SEQ ID NO: 63 or the mature polypeptide of SEQ ID NO: 62;
(r) comprising or consisting of SEQ ID NO: 69 or the mature polypeptide of SEQ ID NO: 68;
(s) comprising or consisting of SEQ ID NO: 75 or the mature polypeptide of SEQ ID NO: 74;
(t) comprising or consisting of SEQ ID NO: 78 or the mature polypeptide of SEQ ID NO: 77;
(u) comprising or consisting of SEQ ID NO: 84 or the mature polypeptide of SEQ ID NO: 83;
(v) comprising or consisting of SEQ ID NO: 87 or the mature polypeptide of SEQ ID NO: 86;
(w) comprising or consisting of SEQ ID NO: 90 or the mature polypeptide of SEQ ID NO: 89;
(x) comprising or consisting of SEQ ID NO: 93 or the mature polypeptide of SEQ ID NO: 92;
(y) comprising or consisting of SEQ ID NO: 96 or the mature polypeptide of SEQ ID NO: 95;
(z) comprising or consisting of SEQ ID NO: 99 or the mature polypeptide of SEQ ID NO: 98;
(aa) comprising or consisting of SEQ ID NO: 108 or the mature polypeptide of SEQ ID NO: 107; and
(bb) comprising or consisting of SEQ ID NO: 111 or the mature polypeptide of SEQ ID NO: 110.

In one embodiment of the invention the composition is a cleaning composition.

In one embodiment of the invention the composition is a cleaning composition comprising
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein said polypeptide is selected from the group consisting of:
(a) a polypeptide having at least 99% sequence identity to the polypeptide of SEQ ID NO: 3;
(b) a polypeptide having at least 62% sequence identity to the polypeptide of SEQ ID NO: 6;
(c) a polypeptide having at least 97% sequence identity to the polypeptide of SEQ ID NO: 12; and
(d) a polypeptide having at least 86% sequence identity to the polypeptide of SEQ ID NO: 21 or 27
(e) a polypeptide having at least 98% sequence identity to the polypeptide of SEQ ID NO: 30;
(f) a polypeptide having 100% sequence identity to the polypeptide of SEQ ID NO: 33 or 48;
(g) a polypeptide having at least 76% sequence identity to the polypeptide of SEQ ID NO: 39 or 51;
(h) a polypeptide having at least 95% sequence identity to the polypeptide of SEQ ID NO: 42;
(i) a polypeptide having at least 79% sequence identity to the polypeptide of SEQ ID NO: 45 or 24;
(j) a polypeptide having at least 94% sequence identity to the polypeptide of SEQ ID NO: 54;
(k) a polypeptide having at least 72% sequence identity to the polypeptide of SEQ ID NO: 57;
(l) a polypeptide having at least 82% sequence identity to the polypeptide of SEQ ID NO: 60;
(m) a polypeptide having at least 75% sequence identity to the polypeptide of SEQ ID NO: 63;
(n) a polypeptide having at least 80% sequence identity to the polypeptide of SEQ ID NO: 69 or 75;
   or selected from a second group consisting of:
(o) a polypeptide having at least 77% sequence identity to the polypeptide of SEQ ID NO: 78;
(p) a polypeptide having at least 94% sequence identity to the polypeptide of SEQ ID NO: 84;
(q) a polypeptide having at least 99% sequence identity to the polypeptide of SEQ ID NO: 87;
(r) a polypeptide having at least 80% sequence identity to the polypeptide of SEQ ID NO: 90; and
(s) a polypeptide having at least 90% sequence identity to the polypeptide of SEQ ID NO: 93;
   or a variant or fragment selected from:
(t) a variant of the polypeptide selected from said first group or said second group wherein the variant has nuclease activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions;
(u) a fragment of the polypeptide of (a), (b), (c), (d), (e), (f), (g), (h), (i), (j), (k), (I), (m), (n), (o), (p), (q), (r) and (s), wherein said fragment has nuclease activity.

(v) a polypeptide having at least 80% sequence identity to the polypeptide of SEQ ID NO: 36;
(w) a polypeptide having at least 80% sequence identity to the polypeptide of SEQ ID NO: 66;
(x) a polypeptide having at least 80% sequence identity to the polypeptide of SEQ ID NO: 72;
(y) a polypeptide having at least 80% sequence identity to the polypeptide of SEQ ID NO: 9;
(z) a polypeptide having at least 80% sequence identity to the polypeptide of SEQ ID NO: 15;
(aa) a polypeptide having at least 80% sequence identity to the polypeptide of SEQ ID NO: 18;
(bb) a polypeptide having at least 80% sequence identity to the polypeptide of SEQ ID NO: 81;
(cc) a polypeptide having at least 80% sequence identity to the polypeptide of SEQ ID NO: 96;
(dd) a polypeptide having at least 80% sequence identity to the polypeptide of SEQ ID NO: 99; and
(ee) a polypeptide comprising the motif [HQ][FILVY]X[GAQS]DX[HTGSA][QVM]P[LFM]H (SEQ ID NO: 102) and/or the motif G[GA]NX[VILFY]X[VLM](SEQ ID NO: 103).

In an embodiment of the invention the composition is a cleaning composition comprising;
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein the polypeptide has a sequence identity to the mature polypeptide of SEQ ID NO: 36 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 36.

In a particular embodiment the composition comprises polypeptides having a sequence identity to the mature polypeptide of SEQ ID NO: 36 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 36.

In a particular embodiment the composition comprises to polypeptides having a sequence identity to the mature polypeptide of SEQ ID NO: 36 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 36.

In a particular embodiment the composition comprises to polypeptides having a sequence identity to the mature polypeptide of SEQ ID NO: 36 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 36.

In a particular embodiment the composition comprises to polypeptides having a sequence identity to the mature polypeptide of SEQ ID NO: 36 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 36.

In a particular embodiment the composition comprises to polypeptides having a sequence identity to the mature polypeptide of SEQ ID NO: 36 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 36.

In a particular embodiment the composition comprises to polypeptides having a sequence identity to the mature polypeptide of SEQ ID NO: 36 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 36.

In a particular embodiment the composition comprises to polypeptides having a sequence identity to the mature polypeptide of SEQ ID NO: 36 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 36.

In an embodiment of the invention the composition is a cleaning composition comprising
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein the polypeptide has a sequence identity to the mature polypeptide of SEQ ID NO: 66 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 66.

In a particular embodiment the composition comprises polypeptides having a sequence identity to the mature polypeptide of SEQ ID NO: 66 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 66.

In a particular embodiment the composition comprises to polypeptides having a sequence identity to the mature polypeptide of SEQ ID NO: 66 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 66.

In a particular embodiment the composition comprises to polypeptides having a sequence identity to the mature polypeptide of SEQ ID NO: 66 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 66.

In a particular embodiment the composition comprises to polypeptides having a sequence identity to the mature polypeptide of SEQ ID NO: 66 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 66.

In a particular embodiment the composition comprises to polypeptides having a sequence identity to the mature polypeptide of SEQ ID NO: 66 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 66.

In a particular embodiment the composition comprises to polypeptides having a sequence identity to the mature polypeptide of SEQ ID NO: 66 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 66.

In a particular embodiment the composition comprises to polypeptides having a sequence identity to the mature polypeptide of SEQ ID NO: 66 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 66.

In an embodiment of the invention the composition is a cleaning composition comprising
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein the polypeptide has a sequence identity to the mature polypeptide of SEQ ID NO: 72 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 72.

In a particular embodiment the composition comprises polypeptides having a sequence identity to the mature polypeptide of SEQ ID NO: 72 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 72.

In a particular embodiment the composition comprises to polypeptides having a sequence identity to the mature polypeptide of SEQ ID NO: 72 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 72.

In a particular embodiment the composition comprises to polypeptides having a sequence identity to the mature polypeptide of SEQ ID NO: 72 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 72.

In a particular embodiment the composition comprises to polypeptides having a sequence identity to the mature polypeptide of SEQ ID NO: 72 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 72.

In a particular embodiment the composition comprises to polypeptides having a sequence identity to the mature polypeptide of SEQ ID NO: 72 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 72.

In a particular embodiment the composition comprises to polypeptides having a sequence identity to the mature polypeptide of SEQ ID NO: 72 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 72.

In a particular embodiment the composition comprises to polypeptides having a sequence identity to the mature polypeptide of SEQ ID NO: 72 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 72.

In an embodiment of the invention the composition is a cleaning composition 15 comprising
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein the polypeptide has a sequence identity to the mature polypeptide of SEQ ID NO: 9 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 9.

In a particular embodiment the composition comprises polypeptides having a sequence identity to the mature polypeptide of SEQ ID NO: 9 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 9.

In a particular embodiment the composition comprises to polypeptides having a sequence identity to the mature polypeptide of SEQ ID NO: 9 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 9.

In a particular embodiment the composition comprises to polypeptides having a sequence identity to the mature polypeptide of SEQ ID NO: 9 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 9.

In a particular embodiment the composition comprises to polypeptides having a sequence identity to the mature polypeptide of SEQ ID NO: 9 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 9.

In a particular embodiment the composition comprises to polypeptides having a sequence identity to the mature polypeptide of SEQ ID NO: 9 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 9.

In a particular embodiment the composition comprises to polypeptides having a sequence identity to the mature polypeptide of SEQ ID NO: 9 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 9.

In a particular embodiment the composition comprises to polypeptides having a sequence identity to the mature polypeptide of SEQ ID NO: 9 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 9.

In an embodiment of the invention the composition is a cleaning composition 25 comprising
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein the polypeptide has a sequence identity to the mature polypeptide of SEQ ID NO: 15 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 15.

In a particular embodiment the composition comprises polypeptides having a sequence identity to the mature polypeptide of SEQ ID NO: 15 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 15.

In a particular embodiment the composition comprises to polypeptides having a sequence identity to the mature polypeptide of SEQ ID NO: 15 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 15.

In a particular embodiment the composition comprises to polypeptides having a sequence identity to the mature polypeptide of SEQ ID NO: 15 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 15.

In a particular embodiment the composition comprises to polypeptides having a sequence identity to the mature polypeptide of SEQ ID NO: 15 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 15.

In a particular embodiment the composition comprises to polypeptides having a sequence identity to the mature polypeptide of SEQ ID NO: 15 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 15.

In a particular embodiment the composition comprises to polypeptides having a sequence identity to the mature polypeptide of SEQ ID NO: 15 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 15.

In a particular embodiment the composition comprises to polypeptides having a sequence identity to the mature polypeptide of SEQ ID NO: 15 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 15.

In an embodiment of the invention the composition is a cleaning composition comprising
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein the polypeptide has a sequence identity to the mature polypeptide of SEQ ID NO: 18 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 18.

In a particular embodiment the composition comprises polypeptides having a sequence identity to the mature polypeptide of SEQ ID NO: 18 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 18.

In a particular embodiment the composition comprises to polypeptides having a sequence identity to the mature polypeptide of SEQ ID NO: 18 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 18.

In a particular embodiment the composition comprises to polypeptides having a sequence identity to the mature polypeptide of SEQ ID NO: 18 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 18.

In a particular embodiment the composition comprises to polypeptides having a sequence identity to the mature polypeptide of SEQ ID NO: 18 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 18.

In a particular embodiment the composition comprises to polypeptides having a sequence identity to the mature polypeptide of SEQ ID NO: 18 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 18.

In a particular embodiment the composition comprises to polypeptides having a sequence identity to the mature polypeptide of SEQ ID NO: 18 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 18.

In a particular embodiment the composition comprises to polypeptides having a sequence identity to the mature polypeptide of SEQ ID NO: 18 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 18.

In an embodiment of the invention the composition is a cleaning composition comprising
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein the polypeptide has a sequence identity to the mature polypeptide of SEQ ID NO: 81 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 81.

In a particular embodiment the composition comprises polypeptides having a sequence identity to the mature polypeptide of SEQ ID NO: 81 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 81.

In a particular embodiment the composition comprises to polypeptides having a sequence identity to the mature polypeptide of SEQ ID NO: 81 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 81.

In a particular embodiment the composition comprises to polypeptides having a sequence identity to the mature polypeptide of SEQ ID NO: 81 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 81.

In a particular embodiment the composition comprises to polypeptides having a sequence identity to the mature polypeptide of SEQ ID NO: 81 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 81.

In a particular embodiment the composition comprises to polypeptides having a sequence identity to the mature polypeptide of SEQ ID NO: 81 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 81.

In a particular embodiment the composition comprises to polypeptides having a sequence identity to the mature polypeptide of SEQ ID NO: 81 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 81.

In a particular embodiment the composition comprises to polypeptides having a sequence identity to the mature polypeptide of SEQ ID NO: 81 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 81.

In an embodiment of the invention the composition is a cleaning composition comprising
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein the polypeptide has a sequence identity to the mature polypeptide of SEQ ID NO: 96 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 96.

In a particular embodiment the composition comprises polypeptides having a sequence identity to the mature polypeptide of SEQ ID NO: 96 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 96.

In a particular embodiment the composition comprises to polypeptides having a sequence identity to the mature polypeptide of SEQ ID NO: 96 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 96.

In a particular embodiment the composition comprises to polypeptides having a sequence identity to the mature polypeptide of SEQ ID NO: 96 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 96.

In a particular embodiment the composition comprises to polypeptides having a sequence identity to the mature polypeptide of SEQ ID NO: 96 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 96.

In a particular embodiment the composition comprises to polypeptides having a sequence identity to the mature polypeptide of SEQ ID NO: 96 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 96.

In a particular embodiment the composition comprises to polypeptides having a sequence identity to the mature polypeptide of SEQ ID NO: 96 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 96.

In a particular embodiment the composition comprises to polypeptides having a sequence identity to the mature polypeptide of SEQ ID NO: 96 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 96.

In an embodiment of the invention the composition is a cleaning composition comprising
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein the polypeptide has a sequence identity to the mature polypeptide of SEQ ID NO: 99 of at least 80%, at least 85%, at least 90%, at least 91%, at least 92%, at least 93%, at least 94%, at least 95%, at least 96%, at least 97%, at least 98%, at least 99%, or 100%, which have nuclease activity. In one aspect, the polypeptides differ by up to 10 amino acids, e.g., 1, 2, 3, 4, 5, 6, 7, 8, 9, or 10, from the mature polypeptide of SEQ ID NO: 99.

In a particular embodiment the composition comprises polypeptides having a sequence identity to the mature polypeptide of SEQ ID NO: 99 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 70% of the nuclease activity of the mature polypeptide of SEQ ID NO: 99.

In a particular embodiment the composition comprises to polypeptides having a sequence identity to the mature polypeptide of SEQ ID NO: 99 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 75% of the nuclease activity of the mature polypeptide of SEQ ID NO: 99.

In a particular embodiment the composition comprises to polypeptides having a sequence identity to the mature polypeptide of SEQ ID NO: 99 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 80% of the nuclease activity of the mature polypeptide of SEQ ID NO: 99.

In a particular embodiment the composition comprises to polypeptides having a sequence identity to the mature polypeptide of SEQ ID NO: 99 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 85% of the nuclease activity of the mature polypeptide of SEQ ID NO: 99.

In a particular embodiment the composition comprises to polypeptides having a sequence identity to the mature polypeptide of SEQ ID NO: 99 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 90% of the nuclease activity of the mature polypeptide of SEQ ID NO: 99.

In a particular embodiment the composition comprises to polypeptides having a sequence identity to the mature polypeptide of SEQ ID NO: 99 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 95% of the nuclease activity of the mature polypeptide of SEQ ID NO: 99.

In a particular embodiment the composition comprises to polypeptides having a sequence identity to the mature polypeptide of SEQ ID NO: 99 of at least 80%, at least 85%, at least 90%, at least 91%, at least 92%, at least 93%, at least 94%, at least 95%, at least 96%, at least 97%, at least 98%, at least 99%, or 100%, and wherein the polypeptide has at least at least 100% of the nuclease activity of the mature polypeptide of SEQ ID NO: 99.

In a preferred embodiment of the invention the cleaning composition comprises one or more surfactants selected from anionic surfactants, nonionic surfactants, cationic surfactants, zwitterionic and mixtures thereof.

In one embodiment the surfactant is an anionic surfactant preferably selected from the group consisting of linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

In one embodiment of the invention, the one or more surfactant is a nonionic surfactant is selected from alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides (FAGA) and combinations thereof.

In one embodiment of the invention the cleaning comprises one or more anionic surfactants and one or more nonionic surfactants.

### Surfactants

The detergent composition may comprise one or more surfactants, which may be anionic and/or cationic and/or non-ionic and/or semi-polar and/or zwitterionic, or a mixture thereof. In a particular embodiment, the detergent composition includes a mixture of one or more nonionic surfactants and one or more anionic surfactants. The surfactant(s) is typically present at a level of from about 0.1% to 60% by weight, such as about 1% to about 40%, or about 3% to about 20%, or about 3% to about 10%. The surfactant(s) is chosen based on the desired cleaning application, and may include any conventional surfactant(s) known in the art.

When included therein the detergent will usually contain from about 1% to about 40% by weight of an anionic surfactant, such as from about 5% to about 30%, including from about 5% to about 15%, or from about 15% to about 20%, or from about 20% to about 25% of an anionic surfactant. Non-limiting examples of anionic surfactants include sulfates and sulfonates, in particular, linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES, also known as alcohol ethoxysulfates or fatty alcohol ether sulfates), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

When included therein the detergent will usually contain from about 1% to about 40% by weigh of a cationic surfactant, for example from about 0.5% to about 30%, in particular from about 1% to about 20%, from about 3% to about 10%, such as from about 3% to about 5%, from about 8% to about 12% or from about 10% to about 12%. Non-limiting examples of cationic surfactants include alkyldimethylethanolamine quat (ADMEAQ), cetyltrimethylammonium bromide (CTAB), dimethyldistearylammonium chloride (DSDMAC), and alkylbenzyldimethylammonium, alkyl quaternary ammonium compounds, alkoxylated quaternary ammonium (AQA) compounds, ester quats, and combinations thereof.

When included therein the detergent will usually contain from about 0.2% to about 40% by weight of a nonionic surfactant, for example from about 0.5% to about 30%, in particular from about 1% to about 20%, from about 3% to about 10%, such as from about 3% to about 5%, from about 8% to about 12%, or from about 10% to about 12%. Non-limiting examples of nonionic surfactants include alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides, FAGA), as well as products available under the trade names SPAN® and TWEEN®, and combinations thereof.

When included therein the detergent will usually contain from about 0.1% to about 10% by weight of a semipolar surfactant. Non-limiting examples of semipolar surfactants include amine oxides (AO) such as alkyldimethylamineoxide, N-(coco alkyl)-N,N-dimethylamine oxide and N-(tallow-alkyl)-N,N-bis(2-hydroxyethyl)amine oxide, and combinations thereof.

When included therein the detergent will usually contain from about 0.1% to about 10% by weight of a zwitterionic surfactant. Non-limiting examples of zwitterionic surfactants include betaines such as alkyldimethylbetaines, sulfobetaines, and combinations thereof.

### Builders and Co-Builders

The detergent composition may contain about 0-65% by weight, such as about 5% to about 50% of a detergent builder or co-builder, or a mixture thereof. In a dish wash detergent, the level of builder is typically 40-65%, particularly 50-65%. The builder and/or co-builder may particularly be a chelating agent that forms water-soluble complexes with Ca and Mg. Any builder and/or co-builder known in the art for use in cleaning detergents may be utilized. Non-limiting examples of builders include zeolites, diphosphates (pyrophosphates), triphosphates such as sodium triphosphate (STP or STPP), carbonates such as sodium carbonate, soluble silicates such as sodium metasilicate, layered silicates (e.g., SKS-6 from Hoechst), ethanolamines such as 2-aminoethan-1-ol (MEA), diethanolamine (DEA, also known as 2,2′-iminodiethan-1-ol), triethanolamine (TEA, also known as 2,2′,2″-nitrilotriethan-1-ol), and (carboxymethyl)inulin (CMI), and combinations thereof.

The detergent composition may also contain 0-50% by weight, such as about 5% to about 30%, of a detergent co-builder. The detergent composition may include a co-builder alone, or in combination with a builder, for example a zeolite builder. Non-limiting examples of co-builders include homopolymers of polyacrylates or copolymers thereof, such as poly(acrylic acid) (PAA) or copoly(acrylic acid/maleic acid) (PAA/PMA). Further non-limiting examples include citrate, chelators such as aminocarboxylates, aminopolycarboxylates and phosphonates, and alkyl- or alkenylsuccinic acid. Additional specific examples include 2,2′,2″-nitrilotriacetic acid (NTA), ethylenediaminetetraacetic acid (EDTA), diethylenetriaminepentaacetic acid (DTPA), iminodisuccinic acid (IDS), ethylenediamine-N,N′-disuccinic acid (EDDS), methylglycinediacetic acid (MGDA), glutamic acid-N,N-diacetic acid (GLDA), 1-hydroxyethane-1,1-diphosphonic acid (HEDP), ethylenediaminetetra(methylenephosphonic acid) (EDTMPA), diethylenetriaminepentakis (methylenephosphonic acid) (DTMPA or DTPMPA), N-(2-hydroxyethyl)iminodiacetic acid (EDG), aspartic acid-N-monoacetic acid (ASMA), aspartic acid-N,N-diacetic acid (ASDA), aspartic acid-N-monopropionic acid (ASMP), iminodisuccinic acid (IDA), N-(2-sulfomethyl)-aspartic acid (SMAS), N-(2-sulfoethyl)-aspartic acid (SEAS), N-(2-sulfomethyl)-glutamic acid (SMGL), N-(2-sulfoethyl)-glutamic acid (SEGL), N-methyliminodiacetic acid (MIDA), α-alanine-N,N-diacetic acid (α-ALDA), serine-N,N-diacetic acid (SEDA), isoserine-N,N-diacetic acid (ISDA), phenylalanine-N,N-diacetic acid (PHDA), anthranilic acid-N,N-diacetic acid (ANDA), sulfanilic acid-N,N-diacetic acid (SLDA), taurine-N,N-diacetic acid (TUDA) and sulfomethyl-N,N-diacetic acid (SMDA), N-(2-hydroxyethyl)ethylenediamine-N,N′,N″-triacetic acid (HEDTA), diethanolglycine (DEG), diethylenetriamine penta(methylenephosphonic acid) (DTPMP), aminotris(methylenephosphonic acid) (ATMP), and combinations and salts thereof. Further exemplary builders and/or co-builders are described in, e.g., WO 09/102854, U.S. Pat. No. 5,977,053.

### Bleaching Systems

The detergent may contain 0-30% by weight, such as about 1% to about 20%, of a bleaching system. Any bleaching system comprising components known in the art for use in cleaning detergents may be utilized. Suitable bleaching system components include sources of hydrogen peroxide; sources of peracids; and bleach catalysts or boosters.

### Sources of Hydrogen Peroxide:

Suitable sources of hydrogen peroxide are inorganic persalts, including alkali metal salts such as sodium percarbonate and sodium perborates (usually mono- or tetrahydrate), and hydrogen peroxide-urea (1/1).

### Sources of Peracids:

Peracids may be (a) incorporated directly as preformed peracids or (b) formed in situ in the wash liquor from hydrogen peroxide and a bleach activator (perhydrolysis) or (c) formed in situ in the wash liquor from hydrogen peroxide and a perhydrolase and a suitable substrate for the latter, e.g., an ester.

a) Suitable preformed peracids include, but are not limited to, peroxycarboxylic acids such as peroxybenzoic acid and its ring-substituted derivatives, peroxy-α-naphthoic acid, peroxyphthalic acid, peroxylauric acid, peroxystearic acid, ε-phthalimidoperoxycaproic acid [phthalimidoperoxyhexanoic acid (PAP)], and o-carboxybenzamidoperoxycaproic acid; aliphatic and aromatic diperoxydicarboxylic acids such as diperoxydodecanedioic acid, diperoxyazelaic acid, diperoxysebacic acid, diperoxybrassylic acid, 2-decyldiperoxybutanedioic acid, and diperoxyphthalic, -isophthalic and -terephthalic acids; perimidic acids; peroxymonosulfuric acid; peroxydisulfuric acid; peroxyphosphoric acid; peroxysilicic acid; and mixtures of said compounds. It is understood that the peracids mentioned may in some cases be best added as suitable salts, such as alkali metal salts (e.g., Oxone®) or alkaline earth-metal salts.

b) Suitable bleach activators include those belonging to the class of esters, amides, imides, nitriles or anhydrides and, where applicable, salts thereof. Suitable examples are tetraacetylethylenediamine (TAED), sodium 4-[(3,5,5-trimethylhexanoyl)oxy]benzene-1-sulfonate (ISONOBS), sodium 4-(dodecanoyloxy)benzene-1-sulfonate (LOBS), sodium 4-(decanoyloxy)benzene-1-sulfonate, 4-(decanoyloxy)benzoic acid (DOBA), sodium 4-(nonanoyloxy)benzene-1-sulfonate (NOBS), and/or those disclosed in WO 98/17767. A particular family of bleach activators of interest was disclosed in EP624154 and particularly preferred in that family is acetyl triethyl citrate (ATC). ATC or a short chain triglyceride like triacetin has the advantage that they are environmentally friendly. Furthermore, acetyl triethyl citrate and triacetin have good hydrolytical stability in the product upon storage and are efficient bleach activators. Finally, ATC is multifunctional, as the citrate released in the perhydrolysis reaction may function as a builder.

### Bleach Catalysts and Boosters

The bleaching system may also include a bleach catalyst or booster.

Some non-limiting examples of bleach catalysts that may be used in the compositions of the present invention include manganese oxalate, manganese acetate, manganese-collagen, cobalt-amine catalysts and manganese triazacyclononane (MnTACN) catalysts; particularly preferred are complexes of manganese with 1,4,7-trimethyl-1,4,7-triazacyclononane (Me3-TACN) or 1,2,4,7-tetramethyl-1,4,7-triazacyclononane (Me4-TACN), in particular Me3-TACN, such as the dinuclear manganese complex [(Me3-TACN)Mn(O)3Mn(Me3-TACN)](PF6)2, and [2,2′,2″-nitrilotris(ethane-1,2-diylazanylylidene-κN-methanylylidene)triphenolato-κ3O]manganese(III). The bleach catalysts may also be other metal compounds, such as iron or cobalt complexes.

In some embodiments, where a source of a peracid is included, an organic bleach catalyst or bleach booster may be used having one of the following formulae:

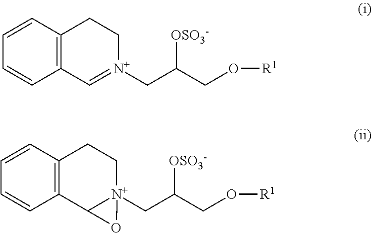

(iii) and mixtures thereof; wherein each R1 is independently a branched alkyl group containing from 9 to 24 carbons or linear alkyl group containing from 11 to 24 carbons, preferably each R1 is independently a branched alkyl group containing from 9 to 18 carbons or linear alkyl group containing from 11 to 18 carbons, more preferably each R1 is independently selected from the group consisting of 2-propylheptyl, 2-butyloctyl, 2-pentylnonyl, 2-hexyldecyl, dodecyl, tetradecyl, hexadecyl, octadecyl, isononyl, isodecyl, isotridecyl and isopentadecyl.

Other exemplary bleaching systems are described, e.g., in WO 2007/087258, WO 2007/087244, WO 2007/087259, EP 1867708 (Vitamin K) and WO 2007/087242. Suitable photobleaches may for example be sulfonated zinc or aluminium phthalocyanines.

### Metal Care Agents

Metal care agents may prevent or reduce the tarnishing, corrosion or oxidation of metals, including aluminium, stainless steel and non-ferrous metals, such as silver and copper.

Suitable examples include one or more of the following:
(a) benzatriazoles, including benzotriazole or bis-benzotriazole and substituted derivatives thereof. Benzotriazole derivatives are those compounds in which the available substitution sites on the aromatic ring are partially or completely substituted. Suitable substituents include linear or branch-chain Ci-C20-alkyl groups (e.g., C1-C20-alkyl groups) and hydroxyl, thio, phenyl or halogen such as fluorine, chlorine, bromine and iodine.
(b) metal salts and complexes chosen from the group consisting of zinc, manganese, titanium, zirconium, hafnium, vanadium, cobalt, gallium and cerium salts and/or complexes, the metals being in one of the oxidation states II, III, IV, V or VI. In one aspect, suitable metal salts and/or metal complexes may be chosen from the group consisting of Mn(II) sulphate, Mn(II) citrate, Mn(II) stearate, Mn(II) acetylacetonate, K\hat{ }TiF6 (e.g., K2TiF6), K\hat{ }ZrF6 (e.g., K2ZrF6), CoSO4, Co(NOs)2 and Ce(NOs)3, zinc salts, for example zinc sulphate, hydrozincite or zinc acetate.
(c) silicates, including sodium or potassium silicate, sodium disilicate, sodium metasilicate, crystalline phyllosilicate and mixtures thereof.

Further suitable organic and inorganic redox-active substances that act as silver/copper corrosion inhibitors are disclosed in WO 94/26860 and WO 94/26859. Preferably the composition of the invention comprises from 0.1 to 5% by weight of the composition of a metal care agent, preferably the metal care agent is a zinc salt.

### Hydrotropes

The detergent may contain 0-10% by weight, for example 0-5% by weight, such as about 0.5 to about 5%, or about 3% to about 5%, of a hydrotrope. Any hydrotrope known in the art for use in detergents may be utilized. Non-limiting examples of hydrotropes include sodium benzenesulfonate, sodium p-toluene sulfonate (STS), sodium xylene sulfonate (SXS), sodium cumene sulfonate (SCS), sodium cymene sulfonate, amine oxides, alcohols and polyglycolethers, sodium hydroxynaphthoate, sodium hydroxynaphthalene sulfonate, sodium ethylhexyl sulfate, and combinations thereof.

### Polymers

The detergent may contain 0-10% by weight, such as 0.5-5%, 2-5%, 0.5-2% or 0.2-1% of a polymer. Any polymer known in the art for use in detergents may be utilized. The polymer may function as a co-builder as mentioned above, or may provide antiredeposition, fiber protection, soil release, dye transfer inhibition, grease cleaning and/or anti-foaming properties. Some polymers may have more than one of the above-mentioned properties and/or more than one of the below-mentioned motifs. Exemplary polymers include (carboxymethyl)cellulose (CMC), poly(vinyl alcohol) (PVA), poly(vinylpyrrolidone) (PVP), poly(ethyleneglycol) or poly(ethylene oxide) (PEG), ethoxylated poly(ethyleneimine), carboxymethyl inulin (CMI), and polycarboxylates such as PAA, PAA/PMA, poly-aspartic acid, and lauryl methacrylate/acrylic acid copolymers, hydrophobically modified CMC (HM-CMC) and silicones, copolymers of terephthalic acid and oligomeric glycols, copolymers of poly(ethylene terephthalate) and poly(oxyethene terephthalate) (PET-POET), PVP, poly(vinylimidazole) (PVI), poly(vinylpyridine-N-oxide) (PVPO or PVPNO) and polyvinylpyrrolidone-vinylimidazole (PVPVI). Suitable examples include PVP-K15, PVP-K30, ChromaBond S-400, ChromaBond S-403E and Chromabond S-100 from Ashland Aqualon, and Sokalan® HP 165, Sokalan® HP 50 (Dispersing agent), Sokalan® HP 53 (Dispersing agent), Sokalan® HP 59 (Dispersing agent), Sokalan® HP 56 (dye transfer inhibitor), Sokalan® HP 66 K (dye transfer inhibitor) from BASF. Further exemplary polymers include sulfonated polycarboxylates, polyethylene oxide and polypropylene oxide (PEO-PPO) and diquaternium ethoxy sulfate. Other exemplary polymers are disclosed in, e.g., WO 2006/130575. Salts of the above-mentioned polymers are also contemplated. Particularly preferred polymer is ethoxylated homopolymer Sokalan® HP 20 from BASF, which helps to prevent redeposition of soil in the wash liquor.

### Fabric Hueing Agents

The detergent compositions of the present invention may also include fabric hueing agents such as dyes or pigments, which when formulated in detergent compositions can deposit onto a fabric when said fabric is contacted with a wash liquor comprising said detergent compositions and thus altering the tint of said fabric through absorption/reflection of visible light. Fluorescent whitening agents emit at least some visible light. In contrast, fabric hueing agents alter the tint of a surface as they absorb at least a portion of the visible light spectrum. Suitable fabric hueing agents include dyes and dye-clay conjugates, and may also include pigments. Suitable dyes include small molecule dyes and polymeric dyes. Suitable small molecule dyes include small molecule dyes selected from the group consisting of dyes falling into the Colour Index (C.I.) classifications of Direct Blue, Direct Red, Direct Violet, Acid Blue, Acid Red, Acid Violet, Basic Blue, Basic Violet and Basic Red, or mixtures thereof, for example as described in WO 2005/03274, WO 2005/03275, WO 2005/03276 and EP 1876226 (hereby incorporated by reference). The detergent composition preferably comprises from about 0.00003 wt % to about 0.2 wt %, from about 0.00008 wt % to about 0.05 wt %, or even from about 0.001 wt % to about 0.04 wt % fabric hueing agent. The composition may comprise from 0.001 wt % to 0.2 wt % fabric hueing agent, this may be especially preferred when the composition is in the form of a unit dose pouch. Suitable hueing agents are also disclosed in, e.g., WO 2007/087257 and WO 2007/087243.

### Enzymes

The detergent additive as well as the detergent composition may comprise one or more additional enzymes such as at least one lipase, cutinase, amylase, carbohydrase, cellulase, pectinase, mannanase, arabinase, galactanase, xylanase, oxidase, e.g., laccase, and/or peroxidase.

In general, the properties of the selected enzyme(s) should be compatible with the selected detergent, (i.e., pH-optimum, compatibility with other enzymatic and non-enzymatic ingredients, etc.), and the enzyme(s) should be present in effective amounts.

### Cellulases

Suitable cellulases include those of bacterial or fungal origin. Chemically modified or protein engineered mutants are included. Suitable cellulases include cellulases from the genera Bacillus, Pseudomonas, Humicola, Fusarium, Thielavia, Acremonium, e.g., the fungal cellulases produced from Humicola insolens, Myceliophthora thermophila and Fusarium oxysporum disclosed in U.S. Pat. Nos. 4,435,307, 5,648,263, 5,691,178, 5,776,757 and WO 89/09259.

Especially suitable cellulases are the alkaline or neutral cellulases having colour care benefits. Examples of such cellulases are cellulases described in EP 0 495 257, EP 0 531 372, WO 96/11262, WO 96/29397, WO 98/08940. Other examples are cellulase variants such as those described in WO 94/07998, EP 0 531 315, U.S. Pat. Nos. 5,457,046, 5,686,593, 5,763,254, WO 95/24471, WO 98/12307 and WO 99/01544.

Other cellulases are endo-beta-1,4-glucanase enzyme having a sequence of at least 97% identity to the amino acid sequence of position 1 to position 773 of SEQ ID NO:2 of WO 2002/099091 or a family 44 xyloglucanase, which a xyloglucanase enzyme having a sequence of at least 60% identity to positions 40-559 of SEQ ID NO: 2 of WO 2001/062903.

Commercially available cellulases include Celluzyme™, Carezyme™ (Novozymes A/S) Carezyme Premium™ (Novozymes A/S), Celluclean™ (Novozymes A/S), Celluclean Classic™ (Novozymes A/S), Cellusoft™ (Novozymes A/S), Whitezyme™ (Novozymes A/S), Clazinase™, and Puradax HA™ (Genencor International Inc.), and KAC-500(B)™ (Kao Corporation).

### Mannanases

Suitable mannanases include those of bacterial or fungal origin. Chemically or genetically modified mutants are included. The mannanase may be an alkaline mannanase of Family 5 or 26. It may be a wild-type from Bacillus or Humicola, particularly B. agaradhaerens, B. licheniformis, B. halodurans, B. clausii, or H. insolens. Suitable mannanases are described in WO 99/64619. A commercially available mannanase is Mannaway (Novozymes A/S).

### Peroxidases/Oxidases

Suitable peroxidases/oxidases include those of plant, bacterial or fungal origin. Chemically modified or protein engineered mutants are included. Examples of useful peroxidases include peroxidases from Coprinus, e.g., from C. cinereus, and variants thereof as those described in WO 93/24618, WO 95/10602, and WO 98/15257. Commercially available peroxidases include Guardzyme™ (Novozymes A/S).

### Lipases and Cutinases

Suitable lipases and cutinases include those of bacterial or fungal origin. Chemically modified or protein engineered mutant enzymes are included. Examples include lipase from Thermomyces, e.g., from T. lanuginosus (previously named Humicola lanuginosa) as described in EP 258068 and EP 305216, cutinase from Humicola, e.g., H. insolens (WO 96/13580), lipase from strains of Pseudomonas (some of these now renamed to Burkholderia), e.g., P. alcaligenes or P. pseudoalcaligenes (EP 218272), P. cepacia (EP 331376), P. sp. strain SD705 (WO 95/06720 & WO 96/27002), P. wisconsinensis (WO 96/12012), GDSL-type Streptomyces lipases (WO 2010/065455), cutinase from Magnaporthe grisea (WO 2010/107560), cutinase from Pseudomonas mendocina (U.S. Pat. No. 5,389,536), lipase from Thermobifida fusca (WO 2011/084412), Geobacillus stearothermophilus lipase (WO 2011/084417), lipase from Bacillus subtilis (WO 2011/084599), and lipase from Streptomyces griseus (WO 2011/150157) and S. pristinaespiralis (WO 2012/137147).

Other examples are lipase variants such as those described in EP 407225, WO 92/05249, WO 94/01541, WO 94/25578, WO 95/14783, WO 95/30744, WO 95/35381, WO 95/22615, WO 96/00292, WO 97/04079, WO 97/07202, WO 00/34450, WO 00/60063, WO 01/92502, WO 2007/87508 and WO 2009/109500.

Preferred commercial lipase products include Lipolase™, Lipex™; Lipolex™ and Lipoclean™ (Novozymes A/S), Lumafast (originally from Genencor) and Lipomax (originally from Gist-Brocades).

Still other examples are lipases sometimes referred to as acyltransferases or perhydrolases, e.g., acyltransferases with homology to Candida antarctica lipase A (WO 2010/111143), acyltransferase from Mycobacterium smegmatis (WO 2005/56782), perhydrolases from the CE 7 family (WO 2009/067279), and variants of the M. smegmatis perhydrolase in particular the S54V variant used in the commercial product Gentle Power Bleach from Huntsman Textile Effects Pte Ltd (WO 2010/100028).

### Amylases

Suitable amylases include alpha-amylases and/or a glucoamylases and may be of bacterial or fungal origin. Chemically modified or protein engineered mutants are included. Amylases include, for example, alpha-amylases obtained from Bacillus, e.g., a special strain of Bacillus licheniformis, described in more detail in GB 1,296,839.

Suitable amylases include amylases having SEQ ID NO: 2 in WO 95/10603 or variants having 90% sequence identity to SEQ ID NO: 3 thereof. Preferred variants are described in WO 94/02597, WO 94/18314, WO 97/43424 and SEQ ID NO: 4 of WO 99/019467, such as variants with substitutions in one or more of the following positions: 15, 23, 105, 106, 124, 128, 133, 154, 156, 178, 179, 181, 188, 190, 197, 201, 202, 207, 208, 209, 211, 243, 264, 304, 305, 391, 408, and 444.

Different suitable amylases include amylases having SEQ ID NO: 6 in WO 02/010355 or variants thereof having 90% sequence identity to SEQ ID NO: 6. Preferred variants of SEQ ID NO: 6 are those having a deletion in positions 181 and 182 and a substitution in position 193.

Other amylases which are suitable are hybrid alpha-amylase comprising residues 1-33 of the alpha-amylase derived from B. amyloliquefaciens shown in SEQ ID NO: 6 of WO 2006/066594 and residues 36-483 of the B. licheniformis alpha-amylase shown in SEQ ID NO: 4 of WO 2006/066594 or variants having 90% sequence identity thereof. Preferred variants of this hybrid alpha-amylase are those having a substitution, a deletion or an insertion in one of more of the following positions: G48, T49, G107, H156, A181, N190, M197, I201, A209 and Q264. Most preferred variants of the hybrid alpha-amylase comprising residues 1-33 of the alpha-amylase derived from B. amyloliquefaciens shown in SEQ ID NO: 6 of WO 2006/066594 and residues 36-483 of SEQ ID NO: 4 are those having the substitutions:
M197T;
H156Y+A181T+N190F+A209V+Q264S; or
G48A+T491+G107A+H156Y+A181T+N190F+I201F+A209V+Q264S.

Further amylases which are suitable are amylases having SEQ ID NO: 6 in WO 99/19467 or variants thereof having 90% sequence identity to SEQ ID NO: 6. Preferred variants of SEQ ID NO: 6 are those having a substitution, a deletion or an insertion in one or more of the following positions: R181, G182, H183, G184, N195, I206, E212, E216 and K269. Particularly preferred amylases are those having deletion in positions R181 and G182, or positions H183 and G184.

Additional amylases which can be used are those having SEQ ID NO: 1, SEQ ID NO: 3, SEQ ID NO: 2 or SEQ ID NO: 7 of WO 96/023873 or variants thereof having 90% sequence identity to SEQ ID NO: 1, SEQ ID NO: 2, SEQ ID NO: 3 or SEQ ID NO: 7. Preferred variants of SEQ ID NO: 1, SEQ ID NO: 2, SEQ ID NO: 3 or SEQ ID NO: 7 are those having a substitution, a deletion or an insertion in one or more of the following positions: 140, 181, 182, 183, 184, 195, 206, 212, 243, 260, 269, 304 and 476, using SEQ ID 2 of WO 96/023873 for numbering. More preferred variants are those having a deletion in two positions selected from 181, 182, 183 and 184, such as 181 and 182, 182 and 183, or positions 183 and 184. Most preferred amylase variants of SEQ ID NO: 1, SEQ ID NO: 2 or SEQ ID NO: 7 are those having a deletion in positions 183 and 184 and a substitution in one or more of positions 140, 195, 206, 243, 260, 304 and 476.

Other amylases which can be used are amylases having SEQ ID NO: 2 of WO 2008/153815, SEQ ID NO: 10 in WO 01/66712 or variants thereof having 90% sequence identity to SEQ ID NO: 2 of WO 2008/153815 or 90% sequence identity to SEQ ID NO: 10 in WO 01/66712. Preferred variants of SEQ ID NO: 10 in WO 01/66712 are those having a substitution, a deletion or an insertion in one of more of the following positions: 176, 177, 178, 179, 190, 201, 207, 211 and 264.

Further suitable amylases are amylases having SEQ ID NO: 2 of WO 2009/061380 or variants having 90% sequence identity to SEQ ID NO: 2 thereof. Preferred variants of SEQ ID NO: 2 are those having a truncation of the C-terminus and/or a substitution, a deletion or an insertion in one of more of the following positions: Q87, Q98, S125, N128, T131, T165, K178, R180, S181, T182, G183, M201, F202, N225, S243, N272, N282, Y305, R309, D319, Q320, Q359, K444 and G475. More preferred variants of SEQ ID NO: 2 are those having the substitution in one of more of the following positions: Q87E,R, Q98R, S125A, N128C, T131I, T165I, K178, T182G, M201L, F202Y, N225E,R, N272E,R, S243Q,A,E,D, Y305R, R309A, Q320R, Q359E, K444E and G475K and/or deletion in position R180 and/or S181 or of T182 and/or G183. Most preferred amylase variants of SEQ ID NO: 2 are those having the substitutions:
N128C+K178L+T182G+Y305R+G475K;
N128C+K178L+T182G+F202Y+Y305R+D319T+G475K;
S125A+N128C+K178L+T182G+Y305R+G475K; or
S125A+Ni128C+T131I+T165I+K178L+T182G+Y305R+G475K
   wherein the variants are C-terminally truncated and optionally further comprises a substitution at position 243 and/or a deletion at position 180 and/or position 181.

Further suitable amylases are amylases having SEQ ID NO: 1 of WO 2013/184577 or variants having 90% sequence identity to SEQ ID NO: 1 thereof. Preferred variants of SEQ ID NO: 1 are those having a substitution, a deletion or an insertion in one of more of the following positions: K176, R178, G179, T180, G181, E187, N192, M199, 1203, S241, R458, T459, D460, G476 and G477. More preferred variants of SEQ ID NO: 1 are those having the substitution in one of more of the following positions: K176L, E187P, N192FYH, M199L, 1203YF, S241QADN, R458N, T459S, D460T, G476K and G477K and/or deletion in position R178 and/or S179 or of T180 and/or G181. Most preferred amylase variants of SEQ ID NO: 1 are those having the substitutions:
E187P+1203Y+G476K
E187P+1203Y+R458N+T459S+D460T+G476K
   wherein the variants optionally further comprise a substitution at position 241 and/or a deletion at position 178 and/or position 179.

Further suitable amylases are amylases having SEQ ID NO: 1 of WO 2010/104675 or variants having 90% sequence identity to SEQ ID NO: 1 thereof. Preferred variants of SEQ ID NO: 1 are those having a substitution, a deletion or an insertion in one of more of the following positions: N21, D97, V128 K177, R179, S180, I181, G182, M200, L204, E242, G477 and G478. More preferred variants of SEQ ID NO: 1 are those having the substitution in one of more of the following positions: N21 D, D97N, V128I K177L, M200L, L204YF, E242QA, G477K and G478K and/or deletion in position R179 and/or S180 or of I181 and/or G182. Most preferred amylase variants of SEQ ID NO: 1 are those having the substitutions:
N21 D+D97N+V128I
   wherein the variants optionally further comprise a substitution at position 200 and/or a deletion at position 180 and/or position 181.

Other suitable amylases are the alpha-amylase having SEQ ID NO: 12 in WO 01/66712 or a variant having at least 90% sequence identity to SEQ ID NO: 12. Preferred amylase variants are those having a substitution, a deletion or an insertion in one of more of the following positions of SEQ ID NO: 12 in WO 01/66712: R28, R118, N174; R181, G182, D183, G184, G186, W189, N195, M202, Y298, N299, K302, S303, N306, R310, N314; R320, H324, E345, Y396, R400, W439, R444, N445, K446, Q449, R458, N471, N484. Particular preferred amylases include variants having a deletion of D183 and G184 and having the substitutions R118K, N195F, R320K and R458K, and a variant additionally having substitutions in one or more position selected from the group: M9, G149, G182, G186, M202, T257, Y295, N299, M323, E345 and A339, most preferred a variant that additionally has substitutions in all these positions.

Other examples are amylase variants such as those described in WO 2011/098531, WO 2013/001078 and WO 2013/001087.

Commercially available amylases are Duramyl™, Termamyl™, Fungamyl™, Stainzyme™, Stainzyme Pus™, Natalase™, Liquozyme X and BAN™ (from Novozymes A/S), and Rapidase™, Purastar™/Effectenz™, Powerase, Preferenz S1000, Preferenz S100 and Preferenz S110 (from Genencor International Inc./DuPont).

### Proteases

Suitable proteases include those of bacterial, fungal, plant, viral or animal origin, e.g., vegetable or microbial origin. Microbial origin is preferred. Chemically modified or protein engineered mutants are included. It may be an alkaline protease, such as a serine protease or a metalloprotease. A serine protease may for example be of the S1 family, such as trypsin, or the S8 family such as subtilisin. A metalloprotease may for example be a thermolysin from, e.g., family M4 or other metalloprotease such as those from M5, M7 or M8 families.

The term “subtilases” refers to a sub-group of serine protease according to Siezen et al., Protein Engng. 4 (1991) 719-737 and Siezen et al. Protein Science 6 (1997) 501-523. Serine proteases are a subgroup of proteases characterized by having a serine in the active site, which forms a covalent adduct with the substrate. The subtilases may be divided into 6 sub-divisions, i.e., the Subtilisin family, the Thermitase family, the Proteinase K family, the Lantibiotic peptidase family, the Kexin family and the Pyrolysin family.

Examples of subtilases are those derived from Bacillus such as Bacillus lentus, Bacillus alkalophilus, Bacillus subtilis, Bacillus amyloliquefaciens, Bacillus pumilus and Bacillus gibsonii described in; U.S. Pat. No. 7,262,042 and WO 2009/021867, and Subtilisin lentus, Subtilisin Novo, subtilisin Carlsberg, Bacillus licheniformis, subtilisin BPN′, subtilisin 309, subtilisin 147 and subtilisin 168 and, e.g., protease PD138 described in (WO 93/18140). Other useful proteases may be those described in WO 01/16285 and WO 02/016547. Examples of trypsin-like proteases are trypsin (e.g., of porcine or bovine origin) and the Fusarium protease described in WO 94/25583 and WO 2005/040372, and the chymotrypsin proteases derived from Cellumonas described in WO 2005/052161 and WO 2005/052146.

A further preferred protease is the alkaline protease from Bacillus lentus DSM 5483, as described for example in WO 95/23221, and variants thereof which are described in WO 92/21760, WO 95/23221, EP 1921147 and EP 1921148.

Examples of metalloproteases are the neutral metalloprotease as described in WO 2007/044993 (Proctor & Gamble/Genencor Int.) such as those derived from Bacillus amyloliquefaciens.

Examples of useful proteases are the variants described in: WO 89/06279 WO 92/19729, WO 96/34946, WO 98/20115, WO 98/20116, WO 99/11768, WO 01/44452, WO 03/006602, WO 2004/03186, WO 2004/041979, WO 2007/006305, WO 2011/036263, WO 2011/036264, especially the variants with substitutions in one or more of the following positions: 3, 4, 9, 15, 24, 27, 42, 55, 59, 60, 66, 74, 85, 96, 97, 98, 99, 100, 101, 102, 104, 116, 118, 121, 126, 127, 128, 154, 156, 157, 158, 161, 164, 176, 179, 182, 185, 188, 189, 193, 198, 199, 200, 203, 206, 211, 212, 216, 218, 226, 229, 230, 239, 246, 255, 256, 268 and 269 wherein the positions correspond to the positions of the Bacillus lentus protease shown in SEQ ID NO: 1 of WO 2016/001449. More preferred the protease variants may comprise one or more of the mutations selected from the group consisting of: S3T, V41, S9R, S9E, A15T, S24G, S24R, K27R, N42R, S55P, G59E, G59D, N60D, N60E, V66A, N74D, S85R, A96S, S97G, S97D, S97A, S97SD, S99E, S99D, S99G, S99M, S99N, S99R, S99H, S101A, V1021, V102Y, V102N, S104A, G116V, G116R, H118D, H118N, A120S, S126L, P127Q, S128A, S154D, A156E, G157D, G157P, S158E, Y161A, R164S, Q176E, N179E, S182E, Q185N, A188P, G189E, V193M, N198D, V1991, Y203W, S206G, L211Q, L211D, N212D, N212S, M216S, A226V, K229L, Q230H, Q239R, N246K, N255W, N255D, N255E, L256E, L256D T268A and R269H. The protease variants are preferably variants of the Bacillus lentus protease shown in SEQ ID NO: 1 of WO 2016/001449, the Bacillus amylolichenifaciens protease (BPN′) shown in SEQ ID NO: 2 of WO 2016/001449. The protease variants preferably have at least 80% sequence identity to SEQ ID NO: 1 or SEQ ID NO: 2 of WO 2016/001449.

Another suitable protease variant is one comprising a substitution at one or more positions corresponding to positions 171, 173, 175, 179, or 180 of SEQ ID NO: 1 of WO 2004/067737, wherein said protease variant has a sequence identity of at least 75% but less than 100% to SEQ ID NO: 1 of WO 2004/067737.

Suitable commercially available protease enzymes include those sold under the trade names Alcalase®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Blaze®, Blaze Evity®100T, Blaze Evity® 125T, Blaze Evity® 150T, Neutrase®, Everlase®, Esperase®, Progress® Uno, Progress® In and Progress® Excel (Novozymes A/S), those sold under the tradename Maxatase®, Maxacal®, Maxapem®, Purafect Ox®, Purafect OxP®, Puramax®, FN2®, FN3®, FN4®, Excellase®, Excellenz P1000™, Excellenz P1250™, Eraser®, Preferenz P100™, Preferenz® P 300, Purafect Prime®, Preferenz P110™, Effectenz P1000™, Purafect®™, Effectenz P1050™, Purafect Ox®™, Effectenz P2000™, Purafast®, Properase®, Opticlean® and Optimase® (Danisco/DuPont), Axapem™ (Gist-Brocases N.V.), BLAP (sequence shown in FIG. 29 of U.S. Pat. No. 5,352,604) and variants hereof (Henkel AG) and KAP (Bacillus alkalophilus subtilisin) from Kao.

### Peroxidases/Oxidases

A peroxidase according to the invention is a peroxidase enzyme comprised by the enzyme classification EC 1.11.1.7, as set out by the Nomenclature Committee of the International Union of Biochemistry and Molecular Biology (IUBMB), or any fragment derived therefrom, exhibiting peroxidase activity.

Suitable peroxidases include those of plant, bacterial or fungal origin. Chemically modified or protein engineered mutants are included. Examples of useful peroxidases include peroxidases from Coprinopsis, e.g., from C. cinerea (EP 179,486), and variants thereof as those described in WO 93/24618, WO 95/10602, and WO 98/15257.

A suitable peroxidase includes a haloperoxidase enzyme, such as chloroperoxidase, bromoperoxidase and compounds exhibiting chloroperoxidase or bromoperoxidase activity. Haloperoxidases are classified according to their specificity for halide ions. Chloroperoxidases (E.C. 1.11.1.10) catalyze formation of hypochlorite from chloride ions. Preferably, the haloperoxidase is a vanadium haloperoxidase, i.e., a vanadate-containing haloperoxidase. Haloperoxidases have been isolated from many different fungi, in particular from the fungus group dematiaceous hyphomycetes, such as Caldariomyces, e.g., C. fumago, Alternaria, Curvularia, e.g., C. verruculosa and C. inaequalis, Drechslera, Ulocladium and Botrytis.

Haloperoxidases have also been isolated from bacteria such as Pseudomonas, e.g., P. pyrrocinia and Streptomyces, e.g., S. aureofaciens.

A suitable oxidase includes, in particular, any laccase enzyme comprised by the enzyme classification EC 1.10.3.2, or any fragment derived therefrom exhibiting laccase activity, or a compound exhibiting a similar activity, such as a catechol oxidase (EC 1.10.3.1), an o-aminophenol oxidase (EC 1.10.3.4), or a bilirubin oxidase (EC 1.3.3.5). Preferred laccase enzymes are enzymes of microbial origin. The enzymes may be derived from plants, bacteria or fungi (including filamentous fungi and yeasts). Suitable examples from fungi include a laccase derivable from a strain of Aspergillus, Neurospora, e.g., N. crassa, Podospora, Botrytis, Collybia, Fomes, Lentinus, Pleurotus, Trametes, e.g., T. villosa and T. versicolor, Rhizoctonia, e.g., R. solani, Coprinopsis, e.g., C. cinerea, C. comatus, C. friesii, and C. plicatilis, Psathyrella, e.g., P. condelleana, Panaeolus, e.g., P. papilionaceus, Myceliophthora, e.g., M. thermophila, Schytalidium, e.g., S. thermophilum, Polyporus, e.g., P. pinsitus, Phlebia, e.g., P. radiata (WO 92/01046), or Coriolus, e.g., C. hirsutus (JP 2238885). Suitable examples from bacteria include a laccase derivable from a strain of Bacillus. A laccase derived from Coprinopsis or Myceliophthora is preferred; in particular, a laccase derived from Coprinopsis cinerea, as disclosed in WO 97/08325; or from Myceliophthora thermophila, as disclosed in WO 95/33836.

### Dispersants

The detergent compositions of the present invention can also contain dispersants. In particular, powdered detergents may comprise dispersants. Suitable water-soluble organic materials include the homo- or co-polymeric acids or their salts, in which the polycarboxylic acid comprises at least two carboxyl radicals separated from each other by not more than two carbon atoms. Suitable dispersants are for example described in Powdered Detergents, Surfactant science series volume 71, Marcel Dekker.

### Dye Transfer Inhibiting Agents

The detergent compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in a subject composition, the dye transfer inhibiting agents may be present at levels from about 0.001% to about 10%, from about 0.01% to about 5% or even from about 0.1% to about 3% by weight of the composition.

### Fluorescent Whitening Agent

The detergent compositions of the present invention will preferably also contain additional components that may tint articles being cleaned, such as fluorescent whitening agent or optical brighteners. Where present the brightener is preferably at a level of about 0.01% to about 0.5%. Any fluorescent whitening agent suitable for use in a laundry detergent composition may be used in the composition of the present invention. The most commonly used fluorescent whitening agents are those belonging to the classes of diaminostilbene-sulfonic acid derivatives, diarylpyrazoline derivatives and bisphenyl-distyryl derivatives. Examples of the diaminostilbene-sulfonic acid derivative type of fluorescent whitening agents include the sodium salts of: 4,4′-bis-(2-diethanolamino-4-anilino-s-triazin-6-ylamino) stilbene-2,2′-disulfonate, 4,4′-bis-(2,4-dianilino-s-triazin-6-ylamino) stilbene-2.2′-disulfonate, 4,4′-bis-(2-anilino-4-(N-methyl-N-2-hydroxy-ethylamino)-s-triazin-6-ylamino) stilbene-2,2′-disulfonate, 4,4′-bis-(4-phenyl-1,2,3-triazol-2-yl)stilbene-2,2′-disulfonate and sodium 5-(2H-naphtho[1,2-d][1,2,3]triazol-2-yl)-2-[(E)-2-phenylvinyl]benzenesulfonate. Preferred fluorescent whitening agents are Tinopal DMS and Tinopal CBS available from Ciba-Geigy AG, Basel, Switzerland. Tinopal DMS is the disodium salt of 4,4′-bis-(2-morpholino-4-anilino-s-triazin-6-ylamino) stilbene-2,2′-disulfonate. Tinopal CBS is the disodium salt of 2,2′-bis-(phenyl-styryl)-disulfonate. Also preferred are fluorescent whitening agents is the commercially available Parawhite KX, supplied by Paramount Minerals and Chemicals, Mumbai, India. Other fluorescers suitable for use in the invention include the 1-3-diaryl pyrazolines and the 7-alkylaminocoumarins. Suitable fluorescent brightener levels include lower levels of from about 0.01, from 0.05, from about 0.1 or even from about 0.2 wt % to upper levels of 0.5 or even 0.75 wt %.

### Soil Release Polymers

The detergent compositions of the present invention may also include one or more soil release polymers which aid the removal of soils from fabrics such as cotton and polyester based fabrics, in particular the removal of hydrophobic soils from polyester based fabrics. The soil release polymers may for example be nonionic or anionic terephthalte based polymers, polyvinyl caprolactam and related copolymers, vinyl graft copolymers, polyester polyamides see for example Chapter 7 in Powdered Detergents, Surfactant science series volume 71, Marcel Dekker, Inc. Another type of soil release polymers is amphiphilic alkoxylated grease cleaning polymers comprising a core structure and a plurality of alkoxylate groups attached to that core structure. The core structure may comprise a polyalkylenimine structure or a polyalkanolamine structure as described in detail in WO 2009/087523 (hereby incorporated by reference). Furthermore, random graft co-polymers are suitable soil release polymers. Suitable graft co-polymers are described in more detail in WO 2007/138054, WO 2006/108856 and WO 2006/113314 (hereby incorporated by reference). Suitable polyethylene glycol polymers include random graft co-polymers comprising: (i) hydrophilic backbone comprising polyethylene glycol; and (ii) side chain(s) selected from the group consisting of: C4-C25 alkyl group, polypropylene, polybutylene, vinyl ester of a saturated C1-C6 mono-carboxylic acid, CI-C 6 alkyl ester of acrylic or methacrylic acid, and mixtures thereof. Suitable polyethylene glycol polymers have a polyethylene glycol backbone with random grafted polyvinyl acetate side chains. The average molecular weight of the polyethylene glycol backbone can be in the range of from 2,000 Da to 20,000 Da, or from 4,000 Da to 8,000 Da. The molecular weight ratio of the polyethylene glycol backbone to the polyvinyl acetate side chains can be in the range of from 1:1 to 1:5, or from 1:1.2 to 1:2. The average number of graft sites per ethylene oxide units can be less than 1, or less than 0.8, the average number of graft sites per ethylene oxide units can be in the range of from 0.5 to 0.9, or the average number of graft sites per ethylene oxide units can be in the range of from 0.1 to 0.5, or from 0.2 to 0.4. A suitable polyethylene glycol polymer is Sokalan HP22. Other soil release polymers are substituted polysaccharide structures especially substituted cellulosic structures such as modified cellulose deriviatives such as those described in EP 1867808 or WO 2003/040279 (both are hereby incorporated by reference). Suitable cellulosic polymers include cellulose, cellulose ethers, cellulose esters, cellulose amides and mixtures thereof. Suitable cellulosic polymers include anionically modified cellulose, nonionically modified cellulose, cationically modified cellulose, zwitterionically modified cellulose, and mixtures thereof. Suitable cellulosic polymers include methyl cellulose, carboxy methyl cellulose, ethyl cellulose, hydroxyl ethyl cellulose, hydroxyl propyl methyl cellulose, ester carboxy methyl cellulose, and mixtures thereof.

### Anti-Redeposition Agents

The detergent compositions of the present invention may also include one or more anti-redeposition agents such as carboxymethylcellulose (CMC), polyvinyl alcohol (PVA), polyvinylpyrrolidone (PVP), polyoxyethylene and/or polyethyleneglycol (PEG), homopolymers of acrylic acid, copolymers of acrylic acid and maleic acid, and ethoxylated polyethyleneimines. The cellulose based polymers described under soil release polymers above may also function as anti-redeposition agents.

### Rheology Modifiers

The detergent compositions of the present invention may also include one or more rheology modifiers, structurants or thickeners, as distinct from viscosity reducing agents. The rheology modifiers are selected from the group consisting of non-polymeric crystalline, hydroxy-functional materials, polymeric rheology modifiers which impart shear thinning characteristics to the aqueous liquid matrix of a liquid detergent composition. The rheology and viscosity of the detergent can be modified and adjusted by methods known in the art, for example as shown in EP 2169040.

Other suitable cleaning composition components include, but are not limited to, anti-shrink agents, anti-wrinkling agents, bactericides, binders, carriers, dyes, enzyme stabilizers, fabric softeners, fillers, foam regulators, hydrotropes, perfumes, pigments, sod suppressors, solvents, and structurants for liquid detergents and/or structure elasticizing agents.

### Formulation of Detergent Products

The detergent composition of the invention may be in any convenient form, e.g., a bar, a homogenous tablet, a tablet having two or more layers, a pouch having one or more compartments, a regular or compact powder, a granule, a paste, a gel, or a regular, compact or concentrated liquid.

Pouches can be configured as single or multicompartments. It can be of any form, shape and material which is suitable for hold the composition, e.g., without allowing the release of the composition to release of the composition from the pouch prior to water contact. The pouch is made from water soluble film which encloses an inner volume. Said inner volume can be divided into compartments of the pouch. Preferred films are polymeric materials preferably polymers which are formed into a film or sheet. Preferred polymers, copolymers or derivates thereof are selected polyacrylates, and water soluble acrylate copolymers, methyl cellulose, carboxy methyl cellulose, sodium dextrin, ethyl cellulose, hydroxyethyl cellulose, hydroxypropyl methyl cellulose, malto dextrin, poly methacrylates, most preferably polyvinyl alcohol copolymers and, hydroxypropyl methyl cellulose (HPMC). Preferably the level of polymer in the film for example PVA is at least about 60%. Preferred average molecular weight will typically be about 20,000 to about 150,000. Films can also be of blended compositions comprising hydrolytically degradable and water soluble polymer blends such as polylactide and polyvinyl alcohol (known under the Trade reference M8630 as sold by MonoSol LLC, Indiana, USA) plus plasticisers like glycerol, ethylene glycerol, propylene glycol, sorbitol and mixtures thereof. The pouches can comprise a solid laundry cleaning composition or part components and/or a liquid cleaning composition or part components separated by the water soluble film. The compartment for liquid components can be different in composition than compartments containing solids: US 2009/0011970.

Detergent ingredients can be separated physically from each other by compartments in water dissolvable pouches or in different layers of tablets. Thereby negative storage interaction between components can be avoided. Different dissolution profiles of each of the compartments can also give rise to delayed dissolution of selected components in the wash solution.

A liquid or gel detergent, which is not unit dosed, may be aqueous, typically containing at least 20% by weight and up to 95% water, such as up to about 70% water, up to about 65% water, up to about 55% water, up to about 45% water, up to about 35% water. Other types of liquids, including without limitation, alkanols, amines, diols, ethers and polyols may be included in an aqueous liquid or gel. An aqueous liquid or gel detergent may contain from 0-30% organic solvent. A liquid or gel detergent may be non-aqueous.

### Granular Detergent Formulations

The compositions of the invention may be formulated as a granule for example as a co-granule that combines one or more enzymes. Each enzyme will then be present in more granules securing a more uniform distribution of enzymes in the detergent. This also reduces the physical segregation of different enzymes due to different particle sizes. Methods for producing multi-enzyme co-granulates for the detergent industry are disclosed in the IP.com disclosure IPCOM000200739D.

Another example of formulation of enzymes by the use of co-granulates are disclosed in WO 2013/188331, which relates to a detergent composition comprising (a) a multi-enzyme co-granule; (b) less than 10 wt zeolite (anhydrous basis); and (c) less than 10 wt phosphate salt (anhydrous basis), wherein said enzyme co-granule comprises from 10 to 98 wt % moisture sink component and the composition additionally comprises from 20 to 80 wt % detergent moisture sink component.

The multi-enzyme co-granule may comprise polypeptide of the invention and (a) one or more enzymes selected from lipases, hemicellulases, proteases, amylases, cellulases, cellobiose dehydrogenases, xylanases, phospho lipases, esterases, cutinases, pectinases, mannanases, pectate lyases, keratinases, reductases, oxidases, phenoloxidases, ligninases, pullulanases, tannases, pentosanases, lichenases glucanases, arabinosidases, hyaluronidase, chondroitinase, amylases, and mixtures thereof.

The compositions may be prepared in accordance with methods known in the art and may be in the form of a liquid or a dry composition. The compositions may be stabilized in accordance with methods known in the art.

Examples are given below of preferred uses of the compositions of the present invention. The dosage of the composition and other conditions under which the composition is used may be determined on the basis of methods known in the art.

### Uses

The polypeptides having nuclease activity may be used for deep cleaning of an item, such as a textile. In one embodiment of the invention relates to the use of polypeptides having nuclease activity for prevention, reduction or removal of malodor. One embodiment of the invention relates to the use of polypeptides having nuclease activity for prevention or reduction of anti-redeposition and/or for improvement of whiteness of a textile subjected to multiple washes. When the biofilm components, e.g., RNA, of the extracellular biofilm matrix are removed or reduced the stickiness caused by biofilm is also reduced, whereby the adherence of soil to the item is prevented, reduced or removed. The polypeptides having nuclease activity therefore reduce the greyness of textiles when applied in the compositions of the invention to a cleaning process such as laundry. The polypeptides having nuclease activity may further be used for preventing, reducing or removing redeposition of soil during cleaning of the item

One aspect of the invention relates to the use of a polypeptide having nuclease activity for cleaning an item by:
(a) preventing, reducing or removing stickiness of the item;
(b) preventing, reducing or removing biofilm or biofilm components from the item;
(c) preventing, reducing or removing redeposition of soil during cleaning of the item;
(d) preventing, reducing or removing adherence of soil to the item;
(e) maintaining or improving whiteness of the item; or
(f) preventing, reducing or removing malodor from the item,
   wherein the item is a textile, and
wherein the polypeptide having nuclease activity selected from the group consisting of SEQ ID NO: 3, SEQ ID NO: 6, SEQ ID NO: 9, SEQ ID NO: 12, SEQ ID NO: 15, SEQ ID NO: 18, SEQ ID NO: 21, SEQ ID NO: 24, SEQ ID NO: 27, SEQ ID NO: 30, SEQ ID NO: 33, SEQ ID NO: 36, SEQ ID NO: 39, SEQ ID NO: 42, SEQ ID NO: 45, SEQ ID NO: 48, SEQ ID NO: 51, SEQ ID NO: 54, SEQ ID NO: 57, SEQ ID NO: 60, SEQ ID NO: 63, SEQ ID NO: 66, SEQ ID NO: 69, SEQ ID NO: 72, SEQ ID NO: 75, SEQ ID NO: 78, SEQ ID NO: 81, SEQ ID NO: 84, SEQ ID NO: 87, SEQ ID NO: 90, SEQ ID NO: 93, SEQ ID NO: 96, SEQ ID NO: 99, SEQ ID NO: 108 and SEQ ID NO: 111, and polypeptides having at least 80% sequence identity hereto: Specific embodiments of the present invention are defined in the following paragraphs:

1. A polypeptide having nuclease activity, selected from a first group consisting of:
(a) a polypeptide having at least 99% sequence identity to the polypeptide of SEQ ID NO: 3;
(b) a polypeptide having at least 62% sequence identity to the polypeptide of SEQ ID NO: 6;
(c) a polypeptide having at least 97% sequence identity to the polypeptide of SEQ ID NO: 12; and
(d) a polypeptide having at least 86% sequence identity to the polypeptide of SEQ ID NO: 21 or 27;
(e) a polypeptide having at least 98% sequence identity to the polypeptide of SEQ ID NO: 30;
(f) a polypeptide having 100% sequence identity to the polypeptide of SEQ ID NO: 33 or 48;
(g) a polypeptide having at least 76% sequence identity to the polypeptide of SEQ ID NO: 39 or 51;
(h) a polypeptide having at least 95% sequence identity to the polypeptide of SEQ ID NO: 42;
(i) a polypeptide having at least 79% sequence identity to the polypeptide of SEQ ID NO: 45 or 24;
(j) a polypeptide having at least 94% sequence identity to the polypeptide of SEQ ID NO: 54;
(k) a polypeptide having at least 72% sequence identity to the polypeptide of SEQ ID NO: 57;
(l) a polypeptide having at least 82% sequence identity to the polypeptide of SEQ ID NO: 60;
(m) a polypeptide having at least 75% sequence identity to the polypeptide of SEQ ID NO: 63; and
(n) a polypeptide having at least 80% sequence identity to the polypeptide of SEQ ID NO: 69 or 75;
   or selected from a second group consisting of:
(o) a polypeptide having at least 77% sequence identity to the polypeptide of SEQ ID NO: 78;
(p) a polypeptide having at least 94% sequence identity to the polypeptide of SEQ ID NO: 84;
(q) a polypeptide having at least 99% sequence identity to the polypeptide of SEQ ID NO: 87;
(r) a polypeptide having at least 80% sequence identity to the polypeptide of SEQ ID NO: 90; and
(s) a polypeptide having at least 90% sequence identity to the polypeptide of SEQ ID NO: 93;
   or a variant or fragment selected from:
(t) a variant of the polypeptide selected from said first group or said second group wherein the variant has nuclease activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions; and
(u) a fragment of the polypeptide of (a), (b), (c), (d), (e), (f), (g), (h), (i), (j), (k), (I), (m), (n), (o), (p), (q), (r) and (s), wherein said fragment has nuclease activity.

2. The polypeptide of paragraph 1, selected from said first group consisting of:
i. a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of any of SEQ ID NO: 6, 24, 39, 45, 51, 57, 63, 69 and 75; and
ii. a polypeptide having at least 95%, at least 96%, at least 97%, at least 98%, at least 99% or 100% sequence identity to the polypeptide of any of SEQ ID NO: 42, 54, 60, 21 and 27.

2. The polypeptide of paragraph 1, selected from said second group consisting of:
i. a polypeptide having at least 80%, at least 85%, at least 90%, at least 91%, at least 92%, at least 93%, at least 94%, at least 95%, at least 96%, at least 97%, at least 98%, at least 99% or 100% sequence identity to the polypeptide of SEQ ID NO: 78 or 90; and
ii. a polypeptide having at least 95%, at least 96%, at least 97%, at least 98%, at least 99% or 100% sequence identity to the polypeptide of SEQ ID NO: 84 or 93.

3. The polypeptide of any of paragraphs 1-3, which is encoded by a polynucleotide having at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to:
i. the mature polypeptide coding sequence of any of SEQ ID NO: 28, 31, 37, 40, 76, 82, 85, 88, and 91; or
ii. the mature polypeptide cDNA coding sequence of any of SEQ ID NO: 1, 4, 10, 19, 22, 25, 43, 46, 49, 52, 55, 58, 61, 67 and 73.

4. The polypeptide of any of paragraphs 1-4, selected from the group consisting of polypeptides:
(a) comprising or consisting of SEQ ID NO: 3 or the mature polypeptide of SEQ ID NO: 2;
(b) comprising or consisting of SEQ ID NO: 6 or the mature polypeptide of SEQ ID NO: 5;
(c) comprising or consisting of SEQ ID NO: 12 or the mature polypeptide of SEQ ID NO: 11;
(d) comprising or consisting of SEQ ID NO: 21 or the mature polypeptide of SEQ ID NO: 20;
(e) comprising or consisting of SEQ ID NO: 24 or the mature polypeptide of SEQ ID NO: 23;
(f) comprising or consisting of SEQ ID NO: 27 or the mature polypeptide of SEQ ID NO: 26;
(g) comprising or consisting of SEQ ID NO: 30 or the mature polypeptide of SEQ ID NO: 29;
(h) comprising or consisting of SEQ ID NO: 33 or the mature polypeptide of SEQ ID NO: 32;
(i) comprising or consisting of SEQ ID NO: 39 or the mature polypeptide of SEQ ID NO: 38;
(j) comprising or consisting of SEQ ID NO: 42 or the mature polypeptide of SEQ ID NO: 41;
(k) comprising or consisting of SEQ ID NO: 45 or the mature polypeptide of SEQ ID NO: 44;
(l) comprising or consisting of SEQ ID NO: 48 or the mature polypeptide of SEQ ID NO: 47;
(m) comprising or consisting of SEQ ID NO: 51 or the mature polypeptide of SEQ ID NO: 50;
(n) comprising or consisting of SEQ ID NO: 54 or the mature polypeptide of SEQ ID NO: 53;
(o) comprising or consisting of SEQ ID NO: 57 or the mature polypeptide of SEQ ID NO: 56;
(p) comprising or consisting of SEQ ID NO: 60 or the mature polypeptide of SEQ ID NO: 59;
(q) comprising or consisting of SEQ ID NO: 63 or the mature polypeptide of SEQ ID NO: 62;
(r) comprising or consisting of SEQ ID NO: 69 or the mature polypeptide of SEQ ID NO: 68;
(s) comprising or consisting of SEQ ID NO: 75 or the mature polypeptide of SEQ ID NO: 74;
(t) comprising or consisting of SEQ ID NO: 78 or the mature polypeptide of SEQ ID NO: 77;
(u) comprising or consisting of SEQ ID NO: 84 or the mature polypeptide of SEQ ID NO: 83;
(v) comprising or consisting of SEQ ID NO: 87 or the mature polypeptide of SEQ ID NO: 86;
(w) comprising or consisting of SEQ ID NO: 90 or the mature polypeptide of SEQ ID NO: 89;
(x) comprising or consisting of SEQ ID NO: 93 or the mature polypeptide of SEQ ID NO: 92;
(y) comprising or consisting of SEQ ID NO: 96 or the mature polypeptide of SEQ ID NO: 95;
(z) comprising or consisting of SEQ ID NO: 99 or the mature polypeptide of SEQ ID NO: 98;
(aa) comprising or consisting of SEQ ID NO: 108 or the mature polypeptide of SEQ ID NO: 107; and
(bb) comprising or consisting of SEQ ID NO: 111 or the mature polypeptide of SEQ ID NO: 110.

6. A polynucleotide encoding the polypeptide of any of paragraphs 1-5.

7. A nucleic acid construct or expression vector comprising the polynucleotide of paragraph 6 operably linked to one or more control sequences that direct the production of the polypeptide in an expression host.

8. A recombinant host cell comprising the polynucleotide of paragraph 6 operably linked to one or more control sequences that direct the production of the polypeptide.

9. A whole broth formulation or cell culture composition comprising the polypeptide of any of paragraphs 1-5.

10. A method of producing the polypeptide of any of paragraphs 1-5, comprising cultivating a cell, which in its wild-type form produces the polypeptide, under conditions conducive for production of the polypeptide, and optionally recovering the polypeptide.

11. A composition comprising the polypeptide of any of paragraphs 1-5.

12. A composition of paragraph 11, wherein the composition is a cleaning composition.

13. The cleaning composition of paragraph 12 comprising:
(a) at least 0.001 ppm of a polypeptide have nuclease activity
(b) one or more surfactants; and
(c) optionally one or more cleaning composition components, preferably selected from surfactants, builders, bleach components, polymers, dispersing agents and additional enzymes,
   wherein said polypeptide is a polypeptide of any of paragraphs 1-5, or said polypeptide is selected from the group consisting of:
(i) a polypeptide having at least 80% sequence identity to the polypeptide of SEQ ID NO: 9;
(ii) a polypeptide having at least 80% sequence identity to the polypeptide of SEQ ID NO: 15;
(iii) a polypeptide having at least 80% sequence identity to the polypeptide of SEQ ID NO: 18;
(iv) a polypeptide having at least 80% sequence identity to the polypeptide of SEQ ID NO: 36;
(v) a polypeptide having at least 80% sequence identity to the polypeptide of SEQ ID NO: 66;
(vi) a polypeptide having at least 80% sequence identity to the polypeptide of SEQ ID NO: 72;
(vii) a polypeptide having at least 80% sequence identity to the polypeptide of SEQ ID NO: 81;
(viii) a polypeptide having at least 80% sequence identity to the polypeptide of SEQ ID NO: 96; and
(ix) a polypeptide having at least 80% sequence identity to the polypeptide of SEQ ID NO: 99;
(x) a polypeptide comprising one or more of the motifs [HQ][FILVY]X[GAQS]DX[HTGSA][QVM]P[LFM]H (SEQ ID NO: 102), G[GA]NX[VILFY]X[VLM](SEQ ID NO: 103) and/or [SADN]R[GS]H (SEQ ID NO: 104), wherein the polypeptide belongs to the Pfam family PF02265 (S1-P1_nuclease), PF01223 (Endonuclease_NS) or PF13930 (Endonuclea_NS_2).

14. The cleaning composition of paragraph 13, wherein the one or more surfactants are selected from anionic surfactants, nonionic surfactants, cationic surfactants, zwitterionic and mixtures thereof.

15. The cleaning composition of paragraph 13 or 14, wherein the one or more surfactant is a anionic surfactant preferably selected from the group consisting of linear alkylbenzenesulfonates (LAS), isomers of LAS, branched alkylbenzenesulfonates (BABS), phenylalkanesulfonates, alpha-olefinsulfonates (AOS), olefin sulfonates, alkene sulfonates, alkane-2,3-diylbis(sulfates), hydroxyalkanesulfonates and disulfonates, alkyl sulfates (AS) such as sodium dodecyl sulfate (SDS), fatty alcohol sulfates (FAS), primary alcohol sulfates (PAS), alcohol ethersulfates (AES or AEOS or FES), secondary alkanesulfonates (SAS), paraffin sulfonates (PS), ester sulfonates, sulfonated fatty acid glycerol esters, alpha-sulfo fatty acid methyl esters (alpha-SFMe or SES) including methyl ester sulfonate (MES), alkyl- or alkenylsuccinic acid, dodecenyl/tetradecenyl succinic acid (DTSA), fatty acid derivatives of amino acids, diesters and monoesters of sulfo-succinic acid or salt of fatty acids (soap), and combinations thereof.

16. The cleaning composition of any of paragraphs 13-15, wherein the one or more surfactant is a nonionic surfactant is selected from alcohol ethoxylates (AE or AEO), alcohol propoxylates, propoxylated fatty alcohols (PFA), alkoxylated fatty acid alkyl esters, such as ethoxylated and/or propoxylated fatty acid alkyl esters, alkylphenol ethoxylates (APE), nonylphenol ethoxylates (NPE), alkylpolyglycosides (APG), alkoxylated amines, fatty acid monoethanolamides (FAM), fatty acid diethanolamides (FADA), ethoxylated fatty acid monoethanolamides (EFAM), propoxylated fatty acid monoethanolamides (PFAM), polyhydroxyalkyl fatty acid amides, or N-acyl N-alkyl derivatives of glucosamine (glucamides, GA, or fatty acid glucamides (FAGA) and combinations thereof.

17. The cleaning composition of paragraph 15 or 16, wherein the composition comprises one or more anionic surfactants and one or more nonionic surfactants.

18. A method for laundering an item comprising:
(a) exposing an item to a wash liquor comprising the polypeptide according to any of paragraphs 1-5 or the composition of any of paragraphs 11-17;
(b) completing at least one wash cycle; and
(c) optionally rinsing the item,
   wherein the item is a textile.

19. Use of a polypeptide of any of paragraphs 1-5, or a cleaning composition of paragraphs 11-17 for cleaning an item by:
(a) preventing, reducing or removing stickiness of the item;
(b) preventing, reducing or removing biofilm or biofilm components from the item;
(c) preventing, reducing or removing redeposition of soil during cleaning of the item;
(d) preventing, reducing or removing adherence of soil to the item;
(e) maintaining or improving whiteness of the item; or
(f) preventing, reducing or removing malodor from the item,
   wherein the item is a textile.

The present invention is further described by the following examples that should not be construed as limiting the scope of the invention.

### EXAMPLES

### Materials and Methods

### Strain

Escherichia coli Top-10 strain purchased from TIANGEN (TIANGEN Biotech Co. Ltd., Beijing, China) was used to propagate our expression vector. Aspergillus oryzae MT3568 strain was used for heterologous expression of the gene encoding a polypeptide having homology with polypeptides with phospholipase activity. A. oryzae MT3568 is an amdS (acetamidase) disrupted gene derivative of A. oryzae JaL355 (WO 02/40694) in which pyrG auxotrophy was restored by disrupting the A. oryzae acetamidase (amdS) gene with the pyrG gene.

### Media

YPM medium was composed of 10 g yeast extract, 20 g Bacto-peptone, 20 g maltose, and deionised water to 1000 ml.

LB plates were composed of 10 g of Bacto-tryptone, 5 g of yeast extract, 10 g of sodium chloride, 15 g of Bacto-agar, and deionised water to 1000 ml.

LB medium was composed of 1 g of Bacto-tryptone, 5 g of yeast extract, and 10 g of sodium chloride, and deionised water to 1000 ml.

COVE sucrose plates were composed of 342 g of sucrose, 20 g of agar powder, 20 ml of COVE salt solution, and deionized water to 1 liter. The medium was sterilized by autoclaving at 15 psi for 15 minutes. The medium was cooled to 60° C. and 10 mM acetamide, 15 mM CsCl, Triton X-100 (50 μl/500 ml) were added.

COVE-2 plate/tube for isolation: 30 g/L sucrose, 20 ml/L COVE salt solution, 10 mM acetamide, 30 g/L noble agar (Difco, Cat #214220).

COVE salt solution was composed of 26 g of MgSO₄·7H₂O, 26 g of KCL, 26 g of KH₂PO₄, 50 ml of COVE trace metal solution, and deionised water to 1000 ml.

COVE trace metal solution was composed of 0.04 g of Na₂B4O₇-10H₂O, 0.4 g of CuSO₄·5H₂O, 1.2 g of FeSO₄·7H₂O, 0.7 g of MnSO₄·H₂O, 0.8 g of Na₂MoO₄·2H₂O, 10 g of ZnSO₄·7H₂O, and deionised water to 1000 ml.

Minimal medium with PNAG-extract (External Polymeric Substances from Staphylococcus xylosus) as carbon, nitrogen and energy source (CN-AG medium) was composed of magnesium sulfate (MgSO₄) 0.2 g, calcium chloride (CaCl₂) 0.02 g, monopotassium phosphate (KH₂PO₄) 1.0 g, dipotassium phosphate (K₂HPO₄) 1.0 g, ferric chloride (FeCl₃) 0.05 g water to 1000 ml and PNAG-extracts.

### Assays

### Test for DNase Activity

Assay Principle: The experimental setup is based on separation of deoxyribonucleic acid (DNA) fragments by anion-exchange chromatography. Binding of DNA to the column is caused by phosphate groups in the backbone of the molecule, and separation of the fragments is induced by an elution step using a linear salt gradient. As a consequence, the order of elution is expected to approximately follow the size of the DNA pieces, the largest molecules being eluted last in the salt gradient due to the highest content of phosphate groups. The elution order from the anion-exchange column is monitored by absorption at 260 nm. Both double- and single-stranded DNA absorb at this wavelength. However, single-stranded DNA will display stronger absorption characteristics due to the hyperchromic effect.

Assay Execution: A suitable substrate for measuring DNase activity is deoxyribonucleic acid sodium salt from salmon testes (D1626 Sigma). This was dissolved at a concentration of 1 mg/mL in 20 mM Tris pH 8.5 buffer. The substrate solution is preferably prepared one day before use and left to condition overnight at 5° C. This ensures a homogenous solution, which is easy to handle and aspirate using pipettes. The candidate enzymes to be tested for activity were diluted to an appropriate concentration using 2 mM MgCl₂+0.01% Triton X-100, and mixed 1:1 with the substrate solution. Then the samples were incubated with shaking at 30° C. (500 rpm) on a thermomixer and the reaction mixture stopped by addition of 200 mM EDTA pH ˜8 in a reaction mixture: EDTA ratio of 9:1. The resulting DNA degradation profile was analyzed using anion-exchange chromatography (e.g., TSKgel® DNA-STAT HPLC column (10×4.6, 5 μm, part #0021962) from Tosoh Bioscience). 10 μL of sample was injected onto a column equilibrated with 20 mM Tris pH 8.5 (Buffer A). The DNA fragments were separated using a flow rate of 0.5 mL/min. in a linear gradient (Buffer B: Buffer A+1 M NaCl): 0-60% B in 15 minutes; 60-100% B in 20 minutes; 100% B for 5 minutes; and finally, 0% B for 5 minutes. The separation of the DNA fragments was monitored at 260 nm using an Agilent 1100 series analytical HPLC equipped with a diode array detector. DNA, EDTA, and sample blanks were included in the setup.

The polypeptides showed DNase activity as shown below.

| Mature protein | | |
|---|---|---|
| SEQ ID NO: | Nuclease family | Origin |
| 15 | S1-P1 | Stenocarpella maydis |
| 27 | S1-P1 | Cadophora fastigiata |
| 9 | S1-P1 | Trichoderma reesei |
| 3 | S1-P1 | Trichoderma hamatum |
| 6 | S1-P1 | Morchella costata |
| 72 | S1-P1 | Acremonium alcalophilum |

### Example 1: Cloning, Expression and Fermentation of Bacterial S1-P1 or EN_NS Nucleases of the Invention

Chromosomal DNA from pure cultures of individual bacterial strains listed in Table 1 and DNA extracted from a metagenome-enrichment of tap water source selected on a minimal medium with PNAG-extract (see Media) at pH7.5 and 30° C. was purified and subjected to full genome sequencing using Illumina sequencing technology. Coding sequences SEQ ID NOs: 28, 31, 34, 37, 40, 43, 76 and 79 encoding for the S1-P1 or EN_NS nucleases SEQ ID NOs: 30, 33, 36, 39, 42, 45, 78 and 81 were identified in those genome assemblies.

TABLE 1

| | | DNA SEQ ID | Mature Protein |
|---|---|---|---|
| Strain | Origin | NO | SEQ ID NO |
| Microbial | Denmark | SEQ ID NO: 28 | SEQ ID NO: 30 |
| enrichment A | | | |
| Lysobacter | Canada | SEQ ID NO: 31 | SEQ ID NO: 33 |
| enzymogenes | | | |
| Pseudoalteromonas | Denmark | SEQ ID NO: 34 | SEQ ID NO: 36 |
| nigrifaciens | | | |
| Vibrio sp. | USA | SEQ ID NO: 37 | SEQ ID NO: 39 |
| Janthinobacterium | Sweden | SEQ ID NO: 40 | SEQ ID NO: 42 |
| agaricidamnosum | | | |
| Massilia aerilata | Sweden | SEQ ID NO: 43 | SEQ ID NO: 45 |
| Bacillus deramificans | Denmark | SEQ ID NO: 76 | SEQ ID NO: 78 |
| Bacillus thuringiensis | Denmark | SEQ ID NO: 79 | SEQ ID NO: 81 |

* Gene nucleotide sequence

The S1-P1 nuclease genes having DNA SEQ ID NO's: 28, 31, 34, 37, 40 and 43 and the EN_NS nuclease genes having DNA SEQ ID NO's: 76 and 79 were cloned and transformed in Bacillus subtilis and expressed as secreted enzymes. For each gene, the sequence encoding the native secretion signal peptide was replaced with a sequence encoding a Bacillus clausii secretion signal (with the following amino acid sequence: SEQ ID NO: 105: MKKPLGKIVASTALLISVAFSSSIASA). Additionally, the gene included a coding sequence for a 6×His tag fused on the carboxy-terminal amino acid residue of the encoded nuclease for purification.

For the purpose of heterologous expression, all S1-P1 nuclease and EN_NS nuclease gene constructs for expression of each of polypeptides having SEQ ID NOs: 30, 33, 36, 39, 42, 45, 78 and 81 were made as linear integration constructs, where the genes were fused by PCR between two Bacillus subtilis homologous chromosomal regions together with a strong promoter and a chloramphenicol resistance marker. The fusion was made by SOE PCR (Horton, R. M., Hunt, H. D., Ho, S. N., Pullen, J. K. and Pease, L. R. (1989) Engineering hybrid genes without the use of restriction enzymes, gene splicing by overlap extension Gene 77: 61-68). The SOE PCR method is also described in patent application WO 2003095658. The genes were expressed under the control of a triple promoter system (as described in WO 99/43835), consisting of the promoters from Bacillus licheniformis alpha-amylase gene (amyL), Bacillus amyloliquefaciens alpha-amylase gene (amyQ), and the Bacillus thuringiensis cryIIIA promoter including stabilizing sequence. The linear PCR constructs were transformed into Bacillus subtilis. Transformants were selected on LB plates supplemented with 6 μg of chloramphenicol per ml.

Recombinant Bacillus subtilis clones comprising an integrated expression construct encoding an S1-P1 nuclease of one of SEQ ID NOs: 30, 33, 36, 39, 42, 45, 78 and 81 were cultivated in 3 L flasks containing 500 ml yeast extract-based medium at 30° C. for 3 days with shaking at 250 rpm. Each of the culture broths was centrifuged at 20,000×g for 20 minutes and the supernatants were carefully decanted from the pelleted material. Each supernatant was filtered using a filtration and EN_NS nucleases were purified from the filtered supernatants as described below.

The filtered supernatants from each fermentation broth were pH adjusted to pH 7.5-8.0, samples were stirred for 30 minutes and filtrated through a 0.2 μm membrane, and the filtrate was applied to a 5 ml HisTrap™ excel column. Prior to loading the samples, the columns were equilibrated in 5 column volumes (CV) of 50 mM TRIS pH 8.0. To remove unbound material, columns were washed with 5 CV of 50 mM HEPES pH 7.0, and elution of bound expressed proteins was performed with 50 mM HEPES pH 7.0+0.75 M imidazole. The eluted proteins were desalted on a ˜50 mL HiPrep™ 26/10 desalting column, equilibrated using 3 CV of 50 mM HEPES pH 7.0+100 mM NaCl. This buffer was also used for transferring the expressed proteins from the loop to the desalting column. The expressed proteins were eluted based on peak fractionation to obtain the sample in one tube. The flow rate was 10 ml/min. An estimate of protein concentration was obtained by absorbance maxima at 280 nm and purity of the samples was evaluated by SDS-PAGE analyses.

### Example 2: Cloning, Expression and Fermentation of Fungal S1-P1 Nucleases of the Invention

Eleven genes encoding nucleases belonging to the PFAM defined protein family designated “S1-P1_nuclease” and having reference number PF02265 (R. D. Finn, et al., Nucleic Acids Research (2016), D44:D279-D285) were cloned from a variety of fungal strains that were isolated from environmental samples or obtained from culture collections as described in Tables 2 and 3 below.

TABLE 2

| | | DNA SEQ | Mature protein |
|---|---|---|---|
| Strain | Origin | ID NO: | SEQ ID NO |
| Morchella costata | Japan | 4 | 6 |
| Penicillium | Japan | 10 | 12 |
| cremeogriseum | | | |
| Cadophora fasfigiata | United | 25 | 27 |
| | Kingdom | | |

* Gene nucleotide sequence

TABLE 3

| | | DNA* SEQ | Mature protein |
|---|---|---|---|
| Strain | Origin | ID NO | SEQ ID NO |
| Trichoderma reesei | Solomon | 64 | 66 |
| | Islands | 7 | 9 |
| Stenocarpella | USA | 13 | 15 |
| maydis | | | |
| Cordyceps | USA | 67 | 69 |
| cardinalis | | 19 | 21 |
| Phialophora | Indonesia | 22 | 24 |
| geniculata | | | |
| Ostropa barbara | Switzerland | 55 | 57 |
| Pyrenochaetopsis | Denmark | 58 | 60 |
| sp. | | | |

*Gene nucleotide sequence

For most of the strains, chromosomal DNA was isolated and used for whole genome sequencing by standard methods known to the person skilled in the art. The whole genome sequences were assembled with either IDBA or SPAdes genome assemblers (Peng et al., 2012, Bioinformatics 28: 1420-1428 and Bankevich et al., 2012, J. Comput. Biol. 19(5):455-477), and genes were annotated on the genomes with the GeneMark 2.3c gene prediction software (Ter-Hovhannisyan et al., 2008, Genome Res. 18(12):1979-1790). For Trichoderma reesei, the assembled genome sequence of RUT-C30 with accession number PRJNA207855 was downloaded from the NCBI genome database (National Center for Biotechnology Information, www.ncbi.nlm.nih.gov/) and annotated with GeneMark 2.3c. Chromosomal DNA was also isolated from RUT-C30 to enable subsequent cloning.

The set of protein sequences predicted from genes annotated on the whole genome sequences were searched for similarity to the PF02265 domain. Eleven proteins identified in this search are listed in the tables 2 and 3 above with their respective SEQ ID NOs for both the nucleotide and peptide sequences. The genes encoding these peptides were cloned by PCR amplification from genomic DNA using gene-specific primers that also append a Kozak translation initiation sequence “TCACC” immediately 5′ of the start codon. The amplified DNA fragments were cloned into the Aspergillus expression vector pMStr57 (WO 2004/032648) that had been digested with BamHI and XhoI.

The cloned nuclease-encoding genes were sequenced and confirmed to be identical to the corresponding genes found in the genome sequence, and transformed into the Aspergillus oryzae strain (WO 2011/057140) by the methods described in Christensen et al., 1988, Biotechnology 6, 1419-1422 and WO 2004/032648. Transformants were selected during regeneration from protoplasts based on the ability, conferred by a selectable marker in the expression vector, to utilize acetamide as a nitrogen source, and were subsequently re-isolated twice under selection.

Production of the recombinant nucleases was evaluated by culturing transformants in 96-well deep-well microtiter plates for 4 days at 30° C. in 0.25 ml of YPG medium (WO 2005/066338) or DAP-4C-1 medium (WO 2012/103350) and monitoring recombinant expression by SDS-PAGE.

For larger-scale production of the recombinant S1-P1 nucleases, a single Aspergillus transformant was selected for each nuclease based on recombinant yield, and most of the transformants were cultured in 500 ml baffled flasks containing 150 ml of DAP-4C-1 medium. The cultures were shaken on a rotary table at 150 RPM at a temperature of 30° C. for 4 days. The recombinant transformant producing the S1-P1 nuclease from Cordyceps cardinalis with SEQ ID NO: 69 was instead cultured at 26° C., and the transformant producing the S1-P1 nuclease from Ostropa barbara was cultivated in DAP-2C-1 medium (WO 04/032648) at 26° C. Culture broth was separated from cellular material by passage through a 0.22 um filtration unit.

Chromatographic purification of recombinantly-expressed S1-P1 nucleases, originating from Morchella costata, Penicillium cremeogriseum, Cadophora fastigiata, Trichoderma reesei, Stenocarpella maydis, Cordyceps cardinalis, Phialophora geniculata and Ostropa barbara, was performed as follows:

The pH of each filtered sample, derived from the respective separated culture both, was adjusted to around pH 7.5 and 1.8M ammonium sulfate (AMS) was added. The samples were applied to a 5 ml HiTrap™ Phenyl (HS) column on an Akta Explorer. Prior to loading, the column had been equilibrated in 5 column volumes (CV) of 50 mM HEPES+1.8 M AMS pH 7. In order to remove unbound material, the column was washed with 5 CV of 50 mM HEPES+1.8 M AMS pH 7. The target proteins were eluted from the column into a 10 ml loop using 50 mM HEPES+20% isopropanol pH 7. From the loop, the samples were loaded onto a desalting column (HiPrep™ 26/10 Desalting), which had been equilibrated with 3CV of 50 mM HEPES+100 mM NaCl pH 7.0. The target proteins were eluted with 50 mM HEPES+100 mM NaCl pH 7.0 and relevant fractions were selected and pooled based on the chromatograms. The flow rate was 5 ml/min. Protein concentration in the final samples was estimated by measuring absorption at 280 nm.

### Example 3: Cloning, Expression and Fermentation of S1-P1 Fungal Nucleases of Penicillium and Trichoderma Origin

The nucleases (S1-P1 nucleases) were derived from fungal strains isolated from environmental samples by standard microbiological isolation techniques. Strains were identified and taxonomy was assigned based on their internal transcribed spacer (ITS) sequences, (see Table 4).

TABLE 4

| | | DNA SEQ | Mature Protein |
|---|---|---|---|
| Strain | Origin | ID NO* | SEQ ID NO |
| Penicillium emersonii | China | 52 | 54 |
| Trichoderma hamatum | China | 1 | 3 |

*Gene nucleotide sequence

Chromosomal DNA from individual strains (Table 1) was isolated by QIAamp DNA Blood Mini Kit (Qiagen, Hilden, Germany). 5 μg of chromosomal DNA were sent for full genome sequencing using Illumina technology. Genome sequencing, the subsequent assembly of reads and the gene discovery (i.e., annotation of gene functions) is known to the person skilled in the art and the service can be purchased commercially. The genome sequences were analyzed for putative nucleases from the PFAM database families PF02265 (S1-P1_nuclease). This analysis identified 2 genes encoding putative nucleases, which were subsequently cloned and recombinantly expressed in Aspergillus oryzae. The 2 genes were amplified by PCR from above isolated fungal genomic DNA. The purified PCR product was cloned into the previously digested expression vector by ligation with an IN-FUSION™ CF Dry-down Cloning Kit (Clontech Laboratories, Inc., Mountain View, CA, USA) according to the manufacturer's instructions. The ligation mixture was used to transform E. coli TOP10 chemically competent cellsCorrect colonies containing SEQ ID NO: 52 and 1 were selected and verified by DNA sequencing (by SinoGenoMax Company Limited, Beijing, China). Colonies comprising SEQ ID NO: 52 and 1 were cultivated overnight in 3 ml of LB medium supplemented with 100 μg of ampicillin per ml. Plasmid DNA was purified using a Qiagen Spin Miniprep kit (Cat. 27106) (QIAGEN GmbH, Hilden, Germany) according to the manufacturer's instructions. Protoplasts of Aspergillus oryzae were prepared according to WO 95/02043. 100 μl of protoplasts were respectively mixed with 2.5-10 μg of each Aspergillus expression vector comprising SEQ ID NO: 52 and 1 and 250 μl of 60% PEG 4000, 10 mM CaCl₂, and 10 mM Tris-HCl pH 7.5 and gently mixed. The mixture was incubated at 37° C. for 30 minutes and the protoplasts were spread onto COVE sucrose plates for selection. After incubation for 4-7 days at 37° C. spores of 4 transformants were inoculated into 3 ml of YPM medium.

After 3 days of cultivation at 30° C., the culture broths were analyzed by SDS-PAGE using Novex® 4-20% Tris-Glycine Gel (Invitrogen Corporation, Carlsbad, CA, USA) to identify the transformants producing the largest amounts of recombinant nucleases.

Spores of the best transformants were spread on COVE-2 plates for re-isolation in order to isolate single colonies. Then a single colony was spread on a COVE-2 tube until sporulation.

Spores from the best expressed transformants were cultivated in 2400 ml of YPM medium in shake flasks for 3 days at a temperature of 30° C. with agitation at 80 rpm. Culture broth was harvested by filtration using a 0.2 μm filter device. The filtered fermentation broth was used for enzyme characterization.

### Example 4: Cloning, Expression and Fermentation of S1-P1 Fungal Nucleases of Aspergillus Origin

TABLE 5

| The wild type gene (A. oryzae) encoding an S1-P1 nuclease (Table 5) | | |
|---|---|---|
| was cloned as follows: | | |
| | DNA SEQ | Mature Protein |
| Strain | ID NO* | SEQ ID NO |
| Aspergillus oryzae | 46 | 48 |

*Gene nucleotide sequence

The wild type S1-P1_nuclease gene was amplified from DNA derived from A. oryzae strain (IF04177) using primers 5′-GACGCGGCCGCACCATGCCGCGCTTACTCCC [SEQ ID NO: 100] and 5′-GACGCGATCGCTCAAGAGGGCTGACTCG [SEQ ID NO: 101] having overhangs with recognition for restriction endonuclease sites, NotI and SgfI, respectively. The amplified DNA (988 base pairs long) was digested with restriction endonucleases NotI and SgfI and the resulting 977-base-pair product was cloned into the corresponding restriction sites of the Aspergillus expression vector pCOIs1202 (described in patent WO 2015/025055 example 2). The resulting vector, named pJaL1349, was transformed into the Aspergillus oryzae strain RUNG237 and expressed, as described.

### Example 5: Cloning, Expression and Fermentation of EN_NS Fungal Nucleases of the Invention

A gene encoding a nuclease belonging to the PFAM defined protein family designated “Endonuclease_NS” and having reference number PF01223 (R. D. Finn, et al., 2016, Nucleic Acids Research D44:D279-D285) was cloned from a strain of Talaromyces leycettanus, CBS398.68, that was originally isolated in England and obtained from CBS-KNAW (Fungal Biodiversity Centre, Utrecht, The Netherlands).

Chromosomal DNA was isolated from the Talaromyces leycettanus strain and the whole genome of this strain was sequenced and assembled by standard methods known to the person skilled in the art. The gene was annotated on the assembled genome sequences with the GeneMark 2.3c gene prediction software (Ter-Hovhannisyan et al., 2008, Genome Res. 18(12):1979-1790).

The predicted protein sequence, assigned SEQ ID NO: 99, based on annotation of the gene on the whole genome sequence, was searched for similarity to the PF01223 domain. The gene encoding this protein was cloned by PCR amplification from genomic DNA using gene-specific primers that also append a Kozak translation initiation sequence “TCACC” immediately 5′ of the start codon. The amplified DNA fragments were cloned into the Aspergillus expression vector pMStr57 (WO 2004/032648) that had been digested with BamHI and XhoI.

The cloned nuclease encoding gene was sequenced and confirmed to be identical to the corresponding gene found in the genome sequence, and was then transformed into the Aspergillus oryzae strain (WO 2011/057140) by the methods described in Christensen et al., 1988, Biotechnology 6: 1419-1422 and WO 2004/032648. Transformants were selected during regeneration from protoplasts based on the ability, conferred by a selectable marker in the expression vector, to utilize acetamide as a nitrogen source, and were subsequently re-isolated twice under selection.

Production of the recombinant Endonuclease_NS nuclease from Talaromyces leycettanus was evaluated by culturing transformants in a 96-well deep-well microtiter plate for 5 days at 30° C. in 0.25 ml of DAP-4C-1 medium (WO 2012/103350) and monitoring recombinant expression by SDS-PAGE.

### Example 6: Construction of Clades and Phylogenetic Trees

### S1 P1 Nuclease Phylogenetic Tree

A phylogenetic tree was constructed from polypeptide sequences of the invention containing an S1-P1_nuclease domain, as defined in PFAM (PF02265, Pfam version 31.0 Finn, 2016, Nucleic Acids Research, Database Issue 44: D279-D285). The phylogenetic tree was constructed from a multiple alignment of mature polypeptide sequences containing at least one S1-P1_nuclease domain. The sequences were aligned using the MUSCLE algorithm version 3.8.31 (Edgar, 2004. Nucleic Acids Research 32(5): 1792-1797), and the trees were constructed using FastTree version 2.1.8 (Price et al., 2010, PloS one 5(3)) and visualized using iTOL (Letunic & Bork, 2007. Bioinformatics 23(1): 127-128).

Polypeptides containing the S1-P1_nuclease domain comprise several conserved motifs. One example is [HQ][FILVY]X[GAQS]DX[HTGSA][QVM]P[LFM]H (SEQ ID NO: 102) situated in positions corresponding to positions 116 to 126 in Trichoderma hamatum (SEQ ID NO: 3), where H at position 116 and D at position 120 are involved in metal ion binding, and H at position 127 is involved in substrate binding.

Another motif contained in the S1_P1_nuclease domain polypeptides is G[GA]NX[VILFY]X[VLM](SEQ ID NO: 103), located at positions 133 to 139 in SEQ ID NO: 3, where G at position 133 is involved in nucleoside binding.

The polypeptides comprising a S1-P1_nuclease domain can be further separated into multiple distinct sub-clusters, or clades, where we define the clades listed below.

### The PLHVG Clade

Polypeptides containing a S1-P1_nuclease domain can be separated into distinct sub-clusters, where sub-clusters are defined by one or more short sequence motifs, as well as by containing an S1-P1_nuclease domain as defined in PFAM (PF02265, Pfam version 31.0 Finn, 2016, Nucleic Acids Research, Database Issue 44: D279-D285).

We denoted one sub-cluster comprising the motif P[LM]H[VA][GA](SEQ ID NO: 112) as the PLHVG clade. It is situated at positions 124 to 128 in Lysobacter enzymogenes (SEQ ID NO: 33). All polypeptide sequences of bacterial origin containing an S1-P1_nuclease domain as well as this motif will be denoted as belonging to the PLHVG clade.

Examples of polypeptides of the PLHVG clade include those with SEQ ID NO: 30, SEQ ID NO: 33, SEQ ID NO: 36, SEQ ID NO: 39, SEQ ID NO: 42 and SEQ ID NO: 45.

### The PLHDE Clade

Another subcluster is defined from the polypeptides containing an S1-P1_nuclease domain. We denoted another sub-cluster comprising the motif PLH[DN]E (SEQ ID NO: 113) as the PLHDE clade. It is situated at positions 124 to 128 in Trichoderma hamatum (SEQ ID NO: 3), where H at position 126 is fully conserved in the clade and is involved in nucleoside binding. All polypeptide sequences of fungal origin containing an S1-P1_nuclease domain as well as this motif will be denoted as belonging to the PLHDE clade.

Examples of polypeptides of the PLHDE clade include those with SEQ ID NO: 3, SEQ ID NO: 9, SEQ ID NO: 12 SEQ ID NO: 15, SEQ ID NO: 18, SEQ ID NO: 21, SEQ ID NO: 48, SEQ ID NO: 51, SEQ ID NO: 54, SEQ ID NO: 60, SEQ ID NO: 66, SEQ ID NO: 69, SEQ ID NO: 72 and SEQ ID NO: 75.

### Endonuclease NS Phylogenetic Tree

A phylogenetic tree was constructed from polypeptide sequences of the invention containing an Endonuclease_NS domain, as defined in PFAM (PF01223, Pfam version 31.0 Finn (2016). Nucleic Acids Research, Database Issue 44: D279-D285). The phylogenetic tree was constructed from a multiple alignment of mature polypeptide sequences containing at least one Endonuclease_NS domain. The sequences were aligned using the MUSCLE algorithm version 3.8.31 (Edgar, 2004. Nucleic Acids Research 32(5): 1792-1797), and the trees were constructed using FastTree version 2.1.8 (Price et al., 2010, PloS one 5(3)) and visualized using iTOL (Letunic & Bork, 2007. Bioinformatics 23(1): 127-128).

Polypeptides containing the Endonuclease_NS domain comprise several conserved motifs. One example is [YV][DN]RGH (SEQ ID NO: 114), situated in positions corresponding to positions 123 to 127 in Penicillium virgatum (SEQ ID NO: 87), where the histidine at position 127 is fully conserved in the clade and putatively involved in substrate binding.

Another example is [SADN]R[GS]H (SEQ ID NO: 104), situated in positions corresponding to positions 154 to 157 in Bacillus deramificans (SEQ ID NO: 81).

The polypeptides comprising an Endonuclease_NS domain can be further separated into multiple distinct sub-clusters, or clades, where we define the clades listed below.

### The YDRGH Clade

Polypeptides containing an Endonuclease_NS domain can be separated into distinct sub-clusters, where sub-clusters are defined by one or more short sequence motifs, as well as containing an Endonuclease_NS domain as defined in PFAM (PF01223, Pfam version 31.0 Finn, 2016, Nucleic Acids Research, Database Issue 44: D279-D285).

We denoted one sub-cluster comprising the motif YDRGHQ[AV](SEQ ID NO: 115) as the YDRGH clade. It is situated at positions 123 to 129 in Penicillium virgatum (SEQ ID NO: 87). All polypeptide sequences of fungal origin containing an Endonuclease_NS domain as well as the above motif will be denoted as belonging to the YDRGH clade.

Examples of polypeptides of the YDRGH clade include those with SEQ ID NO: 87, SEQ ID NO: 93, SEQ ID NO: 96 and SEQ ID NO: 102.

### The DRHGL Clade

Another subcluster was defined from the polypeptides containing an Endonuclease_NS domain. We denoted a sub-cluster comprising the motif [DNA]R[GSC]H[LI](SEQ ID NO: 116) as the DRHGL clade. It is situated at positions 129 to 133 in Streptomyces cirratus (SEQ ID NO: 90). All polypeptide sequences of bacterial origin containing an Endonuclease_NS domain as well as the motif [DNA]R[GSC]H[LI](SEQ ID NO: 116) will be denoted as belonging to the DRHGL clade.

Another motif which may be identified in members of the DRHGL clade is [RIENLG][YF][RHN]V (SEQ ID NO: 117), situated in positions corresponding to positions 191 to 194 in Streptomyces cirratus (SEQ ID NO: 90).

Examples of polypeptides of the DRHGL clade include those with SEQ ID NO: 90, SEQ ID NO: 81, SEQ ID NO: 84, SEQ ID NO: 108 and SEQ ID NO: 111.

### Example 7: DNase Activity of Polypeptides

The cloned and expressed polypeptides listed below were examined for DNase activity. DNase activity was determined by fluorescence using a fluorescence-quenched oligonucleotide probe (relative fluorescence units, RFU). This probe emits a signal after nuclease degradation (DNaseAlert™ kit, Integrated DNA Technologies, Inc., Coralville, Iowa, USA).

Briefly, DNase was diluted in 0.01% Triton solution in Milli-Q to 0.6 ppm and further diluted 1:1 with pH universal buffer (100 mM Acetic Acid, 100 mM MES, 100 mM HEPES, 100 mM Glycin, 2 mM CaCl₂, 2 mM MgCl₂), at pH 2, 3, 4, 5, 6, 7, 8, 9, 10 and 11. The reaction was started by adding 25 μl of the DNaseAlert™ substrate to 100 μl of 0.3 ppm DNase sample. A kinetic curve was measured for 15 min at 22° C. using a Synergy H1 microplate reader, BioTek (excitation 536 nm, emission at 590 nm).

Table 6 below shows the pH optima of the DNase activity, determined by fluorescence after 2 min (given as approximate pH optima, rounded to the nearest whole number pH value). Background in controls that contained DNaseAlert™ fluorescence-quenched oligonucleotide substrate with no enzyme was negligible at the given pH values.

TABLE 6

| SEQ ID NO. | Donor organism | pH optimum |
|---|---|---|
| 15 | Stenocarpella maydis | 6 |
| 21 | Cordyceps cardinalis | 5 |
| 57 | Ostropa barbara | 5 |
| 27 | Cadophora fastigiata | 5 |
| 9 | Trichoderma reesei | 4 |
| 24 | Phialophora geniculata | 5 |
| 18 | Stenocarpella maydis | 4 |
| 12 | Penicillium cremeogriseum | 4 |

### Example 8: DNase Activity of Polypeptides

The cloned and expressed polypeptides listed below were examined for DNase activity. DNase activity was determined by fluorescence using a fluorescence-quenched oligonucleotide probe (relative fluorescence units, RFU). This probe emits a signal after nuclease degradation (DNaseAlert™ kit, Integrated DNA Technologies, Inc., Coralville, Iowa, USA).

Briefly, DNase was diluted in 2 mM MES pH 6.5+0.0025% Brij L23 to approx. 0.2-2 ppm. pH universal buffer (80 mM Acetate, 80 mM MES, 80 mM HEPES, 80 mM TRIS, 10 mM MgCl₂, 0.0050% Brij L23) at pH 4, 5, 6, 7, 8 or 9 was mixed in a 5:3 ratio with the DNaseAlert™ substrate solution (1 vial dissolved in 15 mL Milli-Q water). The reaction was started by adding 20 μL of DNase sample to 80 μL substrate/buffer mixture. A kinetic curve was measured for 15 min at room temperature using a Varioskan® Flash plate reader, Thermo Scientific (excitation at 535 nm, emission at 556 nm).

Table 7 below shows the pH optimum of the DNase activity, given as approximate pH optima (rounded to the nearest whole number pH value). The background in controls that contained DNaseAlert™ fluorescence-quenched oligonucleotide substrate with no enzyme was negligible at the given pH values.

TABLE 7

| SEQ ID NO. | Donor organism | Origin | pH optimum |
|---|---|---|---|
| 108 | Bacillus sp. | Denmark | 7 |
| 111 | Streptococcus infantis | Sweden | 7 |

1. A cleaning composition comprising:
(a) at least 0.001 ppm of a polypeptide have nuclease activity, wherein the polypeptide comprises one or more of the motifs [HQ][FILVY]X[GAQS]DX[HTGSA][QVM]P[LFM]H (SEQ ID NO: 102), G[GA]NX[VILFY]X[VLM](SEQ ID NO: 103), [SADN]R[GS]H (SEQ ID NO: 104), P[LM]H[VA][GA](SEQ ID NO: 112), PLH[DN]E (SEQ ID NO: 113), [YV][DN]RGH (SEQ ID NO: 114), YDRGHQ[AV](SEQ ID NO: 115), [DNA]R[GSC]H[LI](SEQ ID NO: 116) and/or [RIENLG][YF][RHN]V (SEQ ID NO: 117); wherein the polypeptide belongs to the Pfam family PF02265 (S1-P1_nuclease), PF01223 (Endonuclease_NS) or PF13930 (Endonuclea_NS_2); and
(b) one or more surfactants.

2. The cleaning composition of claim 1, wherein the polypeptide is an S1-P1 nuclease that belongs to the Pfam family PF02265 (S1-P1_nuclease), and which comprises the motif [HQ][FILVY]X[GAQS]DX[HTGSA][QVM]P[LFM]H (SEQ ID NO: 102) and/or G[GA]NX[VILFY]X[VLM](SEQ ID NO: 103).

3. The cleaning composition of claim 2, wherein the polypeptide is of bacterial origin and comprises the motif P[LM]H[VA][GA](SEQ ID NO: 112).

4. The cleaning composition of claim 2, wherein the polypeptide is of fungal origin and comprises the motif PLH[DN]E (SEQ ID NO: 113).

5. The cleaning composition of claim 1, wherein the polypeptide is an EN_NS nuclease that belongs to the Pfam family PF01223 (Endonuclease_NS) or PF13930 (Endonuclea_NS_2), and which comprises the motif [SADN]R[GS]H (SEQ ID NO: 104) and/or [YV][DN]RGH (SEQ ID NO: 114), preferably [YV][DN]RGH (SEQ ID NO: 114).

6. The cleaning composition of claim 5, wherein the polypeptide is of fungal origin and comprises the motif YDRGHQ[AV](SEQ ID NO: 115).

7. The cleaning composition of claim 5, wherein the polypeptide is of bacterial origin and comprises the motif [DNA]R[GSC]H[LI](SEQ ID NO: 116) and/or [RIENLG][YF][RHN]V (SEQ ID NO: 117).

8. The cleaning composition of claim 1, wherein said polypeptide is selected from the group consisting of:
(i) a polypeptide having at least 80% sequence identity to the polypeptide of SEQ ID NO: 9;
(ii) a polypeptide having at least 80% sequence identity to the polypeptide of SEQ ID NO: 15;
(iii) a polypeptide having at least 80% sequence identity to the polypeptide of SEQ ID NO: 18;
(iv) a polypeptide having at least 80% sequence identity to the polypeptide of SEQ ID NO: 36;
(v) a polypeptide having at least 80% sequence identity to the polypeptide of SEQ ID NO: 66;
(vi) a polypeptide having at least 80% sequence identity to the polypeptide of SEQ ID NO: 72;
(vii) a polypeptide having at least 80% sequence identity to the polypeptide of SEQ ID NO: 81;
(viii) a polypeptide having at least 80% sequence identity to the polypeptide of SEQ ID NO: 96; and
(ix) a polypeptide having at least 80% sequence identity to the polypeptide of SEQ ID NO: 99.

9. The cleaning composition of claim 1, wherein the polypeptide is selected from a first group consisting of:
(a) a polypeptide having at least 99% sequence identity to the polypeptide of SEQ ID NO: 3;
(b) a polypeptide having at least 62% sequence identity to the polypeptide of SEQ ID NO: 6;
(c) a polypeptide having at least 97% sequence identity to the polypeptide of SEQ ID NO: 12;
(d) a polypeptide having at least 86% sequence identity to the polypeptide of SEQ ID NO: 21 or 27;
(e) a polypeptide having at least 98% sequence identity to the polypeptide of SEQ ID NO: 30;
(f) a polypeptide having 100% sequence identity to the polypeptide of SEQ ID NO: 33 or 48;
(g) a polypeptide having at least 76% sequence identity to the polypeptide of SEQ ID NO: 39 or 51;
(h) a polypeptide having at least 95% sequence identity to the polypeptide of SEQ ID NO: 42;
(i) a polypeptide having at least 79% sequence identity to the polypeptide of SEQ ID NO: 45 or 24;
(j) a polypeptide having at least 94% sequence identity to the polypeptide of SEQ ID NO: 54;
(k) a polypeptide having at least 72% sequence identity to the polypeptide of SEQ ID NO: 57;
(l) a polypeptide having at least 82% sequence identity to the polypeptide of SEQ ID NO: 60;
(m) a polypeptide having at least 75% sequence identity to the polypeptide of SEQ ID NO: 63; and
(n) a polypeptide having at least 80% sequence identity to the polypeptide of SEQ ID NO: 69 or 75;
or is selected from a second group consisting of:
(o) a polypeptide having at least 77% sequence identity to the polypeptide of SEQ ID NO: 78;
(p) a polypeptide having at least 94% sequence identity to the polypeptide of SEQ ID NO: 84;
(q) a polypeptide having at least 99% sequence identity to the polypeptide of SEQ ID NO: 87;
(r) a polypeptide having at least 80% sequence identity to the polypeptide of SEQ ID NO: 90; and
(s) a polypeptide having at least 90% sequence identity to the polypeptide of SEQ ID NO: 93;
or is a variant or fragment selected from:
(t) a variant of the polypeptide selected from said first group or said second group wherein the variant has nuclease activity and comprises one or more amino acid substitutions, and/or one or more amino acid deletions, and/or one or more amino acid insertions or any combination thereof in 1, 2, 3, 4, 5, 6, 7, 8, 9, 10, 11, 12, 13, 14, 15, 16, 17, 18, 19 or 20 positions; and
(u) a fragment of the polypeptide of (a), (b), (c), (d), (e), (f), (g), (h), (i), (j), (k), (l), (m), (n), (o), (p), (q), (r) and (s), wherein said fragment has nuclease activity.

10. The cleaning composition of claim 1, wherein the polypeptide is selected from the group consisting of polypeptides:
(a) comprising or consisting of SEQ ID NO: 3 or the mature polypeptide of SEQ ID NO: 2;
(b) comprising or consisting of SEQ ID NO: 6 or the mature polypeptide of SEQ ID NO: 5;
(c) comprising or consisting of SEQ ID NO: 12 or the mature polypeptide of SEQ ID NO: 11;
(d) comprising or consisting of SEQ ID NO: 21 or the mature polypeptide of SEQ ID NO: 20;
(e) comprising or consisting of SEQ ID NO: 24 or the mature polypeptide of SEQ ID NO: 23;
(f) comprising or consisting of SEQ ID NO: 27 or the mature polypeptide of SEQ ID NO: 26;
(g) comprising or consisting of SEQ ID NO: 30 or the mature polypeptide of SEQ ID NO: 29;
(h) comprising or consisting of SEQ ID NO: 33 or the mature polypeptide of SEQ ID NO: 32;
(i) comprising or consisting of SEQ ID NO: 39 or the mature polypeptide of SEQ ID NO: 38;
(j) comprising or consisting of SEQ ID NO: 42 or the mature polypeptide of SEQ ID NO: 41;
(k) comprising or consisting of SEQ ID NO: 45 or the mature polypeptide of SEQ ID NO: 44;
(l) comprising or consisting of SEQ ID NO: 48 or the mature polypeptide of SEQ ID NO: 47;
(m) comprising or consisting of SEQ ID NO: 51 or the mature polypeptide of SEQ ID NO: 50;
(n) comprising or consisting of SEQ ID NO: 54 or the mature polypeptide of SEQ ID NO: 53;
(o) comprising or consisting of SEQ ID NO: 57 or the mature polypeptide of SEQ ID NO: 56;
(p) comprising or consisting of SEQ ID NO: 60 or the mature polypeptide of SEQ ID NO: 59;
(q) comprising or consisting of SEQ ID NO: 63 or the mature polypeptide of SEQ ID NO: 62;
(r) comprising or consisting of SEQ ID NO: 69 or the mature polypeptide of SEQ ID NO: 68;
(s) comprising or consisting of SEQ ID NO: 75 or the mature polypeptide of SEQ ID NO: 74;
(t) comprising or consisting of SEQ ID NO: 78 or the mature polypeptide of SEQ ID NO: 77;
(u) comprising or consisting of SEQ ID NO: 84 or the mature polypeptide of SEQ ID NO: 83;
(v) comprising or consisting of SEQ ID NO: 87 or the mature polypeptide of SEQ ID NO: 86;
(w) comprising or consisting of SEQ ID NO: 90 or the mature polypeptide of SEQ ID NO: 89;
(x) comprising or consisting of SEQ ID NO: 93 or the mature polypeptide of SEQ ID NO: 92;
(y) comprising or consisting of SEQ ID NO: 96 or the mature polypeptide of SEQ ID NO: 95;
(z) comprising or consisting of SEQ ID NO: 99 or the mature polypeptide of SEQ ID NO: 98;
(aa) comprising or consisting of SEQ ID NO: 108 or the mature polypeptide of SEQ ID NO: 107; and
(bb) comprising or consisting of SEQ ID NO: 111 or the mature polypeptide of SEQ ID NO: 110.

11-13. (canceled)

14. The cleaning composition of claim 1, wherein the one or more surfactants are selected from anionic surfactants, nonionic surfactants, cationic surfactants, zwitterionic and mixtures thereof.

15. The cleaning composition of claim 14, wherein the composition comprises at least one anionic surfactant, at least one nonionic surfactant, or both.

16. A polypeptide having nuclease activity, wherein the polypeptide is as defined in claim 1.

17. A polynucleotide encoding the polypeptide of claim 16.

18. A nucleic acid construct or expression vector comprising the polynucleotide of claim 17 operably linked to one or more control sequences that direct production of the polypeptide in an expression host.

19. A recombinant host cell comprising the polynucleotide of claim 17 operably linked to one or more control sequences that direct production of the polypeptide.

20-21. (canceled)

22. A method for laundering an item, comprising the steps of:
(a) exposing an item to a wash liquor comprising the cleaning composition of claim 1;
(b) completing at least one wash cycle; and
(c) optionally rinsing the item,
wherein the item is a textile.

23. (canceled)
